SEQUENCE LISTING (83 sequences)

SEQ ID NO: 1 - protein, 31 aa, Homo sapiens
SSVPPQDTAPYSCHVQHSSLAQPLVVPWEAS

SEQ ID NO: 2 - protein, 14 aa, Homo sapiens
PQTTAPRRARPRRS

SEQ ID NO: 3 - protein, 39 aa, Homo sapiens
EVHIKKKTKQTLTNFQMGLLVRGREWPCPGCAACLSKLP

SEQ ID NO: 4 - protein, 150 aa, Homo sapiens
SSGSGESRLQHSPSQSYLCIPFPRGEDGDGPSSDGIHEEPTPVNSATSTPQLTPTNSLKRGGAHHRRCEVALLGCGAVLAATGLGFDLLEAGKCQLLPLEEPEPPAREEKKRREGLFQRSSRPRRSTSPPSRKLFKKEEHQACGRTRVTS

SEQ ID NO: 5 - protein, 27 aa, Homo sapiens
VSGSQRVKYLLVNPLQKKFINPCYRGF

SEQ ID NO: 6 - protein, 22 aa, Homo sapiens
PKCRLQRQYTGKGGVGFVYEGV

SEQ ID NO: 7 - protein, 25 aa, Homo sapiens
PVSSSGSYSTPIRKSLRRAAPPFRA

SEQ ID NO: 8 - protein, 20 aa, Homo sapiens
NSGASGSRNFSSCSAEDFEK

SEQ ID NO: 9 - protein, 84 aa, Homo sapiens
KAECFKNLIVKKQKSLCSGFKEHLNEASILAQVSVSSSKRVWKSWENLISSFMVWNPAHLIISIPNLEKTSDLSMMSKLAAALE

SEQ ID NO: 10 - protein, 76 aa, Homo sapiens
NTLVTYDMVPEPKIIDAALRACRRLNDFASTVRILEVVKDKAGPHKEIYPYVIQELRPTLNELGISTPEELGLDKV

SEQ ID NO: 11 - protein, 25 aa, Homo sapiens
CSKHSSLLLFSSCKQLKIFKIKFTL

SEQ ID NO: 12 - protein, 55 aa, Homo sapiens
APRTRTLRARRSPRMEIAQKWMMKTVKEEEWNVWMKCPILKNSLPISKINFIKND

SEQ ID NO: 13 - protein, 11 aa, Homo sapiens
LVSILLTKTIY

SEQ ID NO: 14 - protein, 22 aa, Homo sapiens
LICISLMANDVEHLFMFICHLS

SEQ ID NO: 15 - protein, 115 aa, Homo sapiens
SSCSEYNVRVASRYFKGPELLVDYQMYDYSLDMWSLGCMLASMIFRREPFFHGQDNYDQLVRIAKVLGTEELYGYLKKYHIDLDPHFNDILGQHSRKRWENLSIVRTDTLSALRP

SEQ ID NO: 16 - protein, 11 aa, Homo sapiens
SAFRGYLANNK

SEQ ID NO: 17 - protein, 64 aa, Homo sapiens
QARLFIFITQKSFIFLFSFLTLCLCLQHFHNDFLLLDKESTLDPVTNTFSTHGTKTLLLTSLFL

SEQ ID NO: 18 - protein, 19 aa, Homo sapiens
VSTFLSRVGRVSLLNFLPF

SEQ ID NO: 19 - protein, 89 aa, Homo sapiens
AARLGPSLECWAAGSAGPFTAHRRPAQVGRPLSLARGPSWSWRRCWSPGRCPSAPWRAGSRPAASCPDWIPGPQGLWLHRNPTSVRPAR

SEQ ID NO: 20 - protein, 22 aa, Homo sapiens
GGGRGAGGGRGAGAGGGRPEAA

SEQ ID NO: 21 - protein, 92 aa, Homo sapiens
  MOD_RES (41)..(41): Any amino acid
QRSGRDNGDVGAGAPFRLSSTSQPRRIKPIAPPPRAPSPEXGAGGGGGGRGGGGGGPGGGGVGGRGGGGGGGGRGAGGGRGAGAGGGRPEAA

SEQ ID NO: 22 - protein, 30 aa, Homo sapiens
GGRGGGGGGGGRGAGGGRGAGAGGGRPEAA

SEQ ID NO: 23 - protein, 32 aa, Homo sapiens
GVGGRGGGGGGGGRGAGGGRGAGAGGGRPEAA

SEQ ID NO: 24 - protein, 22 aa, Homo sapiens
REMVPRMRRTSRASIHHIKPTE

SEQ ID NO: 25 - protein, 76 aa, Homo sapiens
LAHRPPCAEPDPGQRMELPAPVPRPRGASKPRDGTSSHCDMPNCQHPQGPGPAGEIRSRCRSCWLRAVRCNPWLGR

SEQ ID NO: 26 - DNA, 463 bp, Homo sapiens
  modified_base (410)..(410): a, c, t, g, unknown or other
tcaagcgtgcccccgcaggacacagccccctactcctgccacgtgcagcacagcagcctggcccagcccctcgtggtgccctgggaggccagctaggaagcaagggttggaggcaatgtgggatctcagacccagtagctgcccttcctgcctgatgtgggagctgaaccacagaaatcacagtcaatggatccacaaggcctgaggagcagtgtggggggacagacaggaggtggatttggagaccgaagactgggatgcctgtcttgagtagacttggacccaaaaaatcatctcaccttgagcccacccccaccccattgtctaatctgtagaagccggaagcttgcggccgcactcgagtaactagttaaccccttggggcctctaaacgggtcttgaggggttanctngttnctcgngtgcggccgcnngcttccggcttctncngnttngncnntgn

SEQ ID NO: 27 - DNA, 221 bp, Homo sapiens
agctttcgctagagacgcctccataagtcacttgcccgttggcccccacgatcggggtcggttgctcgcagggctgagcagagatgtgccaggagggttgttctcacgcaagaggacgctgtactcctgctgctggaaagtaggcgcctcgtcgttgacgtcagcgacactgacggtcaggacctgcgtggccgagcgcggcggggagccgtggtctgagg

SEQ ID NO: 28 - DNA, 281 bp, Homo sapiens
gggaagtccacattaaaaagaaaacaaaacaaaccctaactaacttccaaatgggtctcctggtgcgggggcgtgagtggccgtgccctgggtgtgctgcctgtctgagcaagcttccctagctgtggaaccccgggccccctgctgcgggctctgccttggtgtcatgcctgctgcacccccgtttccactgacgtgccgtctgtggctatgggggtggtcactggaatgacggtcactccagacgtcagccggcagggatgcagcaggctggccgcgca

SEQ ID NO: 29 - DNA, 532 bp, Homo sapiens
  modified_base (490)..(490): a, c, t, g, unknown or other
tcaagcgggagtggagagagtcgcctacagcattcacccagccagtcctacctctgtatcccattccctcgtggagaggatggcgatggcccctccagtgatggaatccatgaggagcccaccccagtcaactcggccacgagtacccctcagctgacgccaaccaacagcctcaagcggggcggtgcccaccaccgccgctgcgaggtggctctgctcggctgtggggctgttctggcagccacaggcctagggtttgacttgctggaagctggcaagtgccagctgcttcccctggaggagcctgagccaccagcccgggaggagaagaaaagacgggagggtctttttcagaggtccagccgtcctcgtcggagcaccagccccccatcccgaaagcttttcaagaaggaggagcaccaagcttgcggccgcactcgagtaactagttaaccccttggggcctctaaacgggtcttgagggggttanctngttactcgngtgcggccgcnngcttggtgctcnncnttn

SEQ ID NO: 30 - DNA, 401 bp, Homo sapiens
gagatgtaagcggctcacaaagggtgaaatatttactagttaacccccttgcagaaaaagttatcaacccttgctacagaggattttaaaaaataaaatacagcttgttctatctttagcatctaactggggaaaagaatcataacatgtgaaagaataaataagaaattgtgctaacagtaaggagtgttatatgaaatattacctgaagaacatgaaacttgaacttgctagagatagagaatatttaaagaggctaagcagagcatttcagggaaagggcaagaagaagcctgggttgtgtgtgaggaaatcagctgacagaggaggagactattaaggaagcataaggaaagaaagacaaaaaattggggtaaaaatatgtacggctttgaaagctt

SEQ ID NO: 31 - DNA, 547 bp, Homo sapiens
gaaatgtagactgcaaaggcagtatacaggaaaaggtggagtgggttttgtttatgagggtgtctgaaaactaaaattgagcgggatatcatggtatagttggacagtattggtccttcacactttggccatattgtataatggagcttttaccaaagatgtatgagaagtgtaagactataaaaaaatgaactattcaaagtaaaactcttaacaaacattttacttaaagcagatgcaaaagggtattctcatgtaggctcctgttggtgcagagggatttttttgatttcaggatacaactaaagtacgaagttctcagtttcactttagtagaaagagctctagaaatgaggctgataaacacatctaagaacactggttgctttctaaaatttccaaagctccaccataaatgtaatttttagtgtttcaaatgattgcattttaaagtatataaatatgggttatccaatatcaatgctatagtaacatcctgaaacaaaacaagcacaaaggtataaatgcctaaactggaggaagcttg

SEQ ID NO: 32 - DNA, 267 bp, Homo sapiens
tggaggagaggctgggctgccccaagcccctgctcagggcctcagaagccatacaccttcactctgattgtgctcatcaaggcccagcatgcaggaggctcaaagtagcttttggcttgggtgttgacgagaagagaggtaacctggggtcattcttgacacgttccagccacctccggttggcctcaattatgccctgaaaggtggtgctgcccgcctcagggacttgcgaatgggagtgctgtaggagccggagctgctcactgg

SEQ ID NO: 33 - DNA, 306 bp, Homo sapiens
tcggcataaagtacctcctggaaggaaccgacagtctttacaacagtcaccatatgcacactcagcaaatgatttaagcttacaggtacttccttcgcagcaagggtccaattcacattcctttggagtaccacagtcacactcttccccagcgtccaccaacttattaccacaggagggagcactataggcttcatcaggctttggaatattaagaaggcagtttcctcctttatttaaagttacttctcaaagtcctctgcactgcaactgctaaagtttctggaacccgatgctcctgaattc

SEQ ID NO: 34 - DNA, 235 bp, Homo sapiens
aagcagagtgctttaaaaatttgatagtaaaaaagcaaaaatctctgtgctctggttttaaggaacatttgaatgaggcaagcattttagcacaggtttctgtttcaagttcaaagagagtctggaaaagttgggaaaatttaatatcatcttttatggtgtggaatcctgcccatttgattatttctatcccaaatcttgaaaaaacatcagacttatctatgatgtcaaagct

SEQ ID NO: 35 - DNA, 263 bp, Homo sapiens
aacacacttgttacctatgatatggttccagagcccaaaatcattgatgctgctttgcgggcatgcagacggttaaatgattttgctagtacagttcgtatcctagaggttgttaaggacaaagcaggacctcataaggaaatctacccctatgtcatccaggaacttagaccaactttaaatgaactgggaatctccactccggaggaactgggccttgacaaagtgtaaccgcataataaaagggaaatgagtttgaactg

SEQ ID NO: 36 - DNA, 352 bp, Homo sapiens
atgttctaagcacagctctcttctcctattttcatcctgcaagcaactcaaaatatttaaaataaagtttacattgtagttattttcaaatctttgcttgataagtattaagaaatattggacttgctgccgtaatttaaagctctgttgattttgtttccgtttggatttttgggggaggggagcactgtgtttatgctggaatatgaagtctgagaccttcggtgctgggaacacacaagagttgttgaaagttgacaagcagactgcgcatgtctctgatgctttgtatcattcttgagcaatcgctcggtccgtggacaataaacagtattatcaaagagaaaaaaaa

SEQ ID NO: 37 - DNA, 343 bp, Homo sapiens
tcgtcgaggctcctgctcctgtgactctcgagcagccagaggctcctacctctatcgagtctttacctactacttctgacactttcttcttcttaccttacaaacctactttacaggttagaactttttgtcaaatggctagagtttctagttgaaatatttcttgctaattcagtccacctacgttttgatgttcttcagtatcgaccttttcgtggtcttatgaaccttggcgaccgttgaaatgtccttttatacgtttaagcatgtttccatcgtccttagatatctctcgagacgaatcttagacatttcttgtttatacttacactttaagttcgaa

SEQ ID NO: 38 - DNA, 373 bp, Homo sapiens
ttactgttacctgatcaatgacagagccttctgaggacattccaagacagtatacagtcctgtggtctccttggaaatccgtctagttaacatttcaagggcaataccgtgttggttttgactggatattcatataaactttttaaagagttgagtgatagagctaacccttatctgtaagttttgaatttatattgtttcatcccatgtacaaaaccattttttcctacaaatagtttgggttttgttgttgtttcttttttttgttttgtttttgttttttttttttttgcgttcgtggggttgtaaaagaaaagaaagcagaatgttttatcatggtttttgcttcagcggctttaggacaaattaaaag

SEQ ID NO: 39 - DNA, 319 bp, Homo sapiens
cctgggcagtgattaggtcataaaggtggagtcctcatggatgggattagtgtctttataaaagagacctttgccatgtgaggttacagtgagaagacatctgtctatgaagaaagtgggccctcaccaaacacagtctgctggcactttgcacttcaactccccagcttccagaactgtaaggaatataagtctgttgttggtaagccacccggtctatgatattttgttatagcagcccaaacagactaagacaggtgacaaataaacatgaaaagatgttcaacatcattagccattagggaaatgcagattaaaa

SEQ ID NO: 40 - DNA, 522 bp, Homo sapiens
tcatcctgctcggagtacaatgttcgtgtagcctcaaggtacttcaagggaccagagctcctcgtggactatcagatgtatgattatagcttggacatgtggagtttgggctgtatgttagcaagcatgatctttcgaagggaaccattcttccatggacaggacaactatgaccagcttgttcgcattgccaaggttctgggtacagaagaactgtatgggtatctgaagaagtatcacatagacctagatccacacttcaacgatatcctgggacaacattcacggaaacgctgggaaaacttatccatagtgagaacagacaccttgtcagccctgaggccctagatcttctggacaaacttctgcgatacgaccatcaacagagactgactgccaaagaggccatggagcacccatacttctaccctgtggtgaaggagcagtcccagccttgtgcagacaatgctgtgctttccagtggtctcacggcagcacgatgaagactggaaagcgacgggt

SEQ ID NO: 41 - DNA, 292 bp, Homo sapiens
taagctttcatcttccccaaccctgatgtcttcctattctcactgatccccctactgactcagcttcacgcttcttgattatacctctctcctgtagaaaagccttggctggctctcctttaggatgagaataaatccgaaatccttagtgtagcatttagaagtcctatctcccacttgtttcttaatattctcttctctaacaccgaacttgtttcaagcctcttttccaacacatgatttcttctattctaaatcaatttatttattatttgctaaatagcccctaaac

SEQ ID NO: 42 - DNA, 326 bp, Homo sapiens
  modified_base (60)..(60): a, c, t, g, unknown or other
ttctcgagtgcggccgcagcttgggtatggagacatatcatataagtaatgctagggtcngtggtaggaagttttttcataggaggtgtatgagttggtcgtagcggaatcgggggtatgctgttcgaattcataagaacagggaggttagaagtagggtcttggttccatgtgtgctaaatgtgttcgtgacaggatcaagcgtgctttccttatcgaggagcagaaaatcgttgtgaaagtgttgaaggcacaagcacagagtcagaaagctaaataaaaaaatgaaacttttttgagtaataaaaatgaaaagacgcgcttga

SEQ ID NO: 43 - DNA, 644 bp, Homo sapiens
gtttccacattcttgtcaagggttggtagggtcagtcttttaaatttcttgccattttagtgactgtgcattggtatttcattgtggtttatttgcatgatgactaatgctcaacaccaactaatcatgttgagtatttttaatgtgcttatttgccactcatatatcttctttgatgaagtgtctcttcaaatattttgcccatttaaaaactgtattgattcttattattgaattgcaataattctttctatccggatatatatcctttgccagatatgtgtattacaaatgttttctcctagccttccacctcagcctcccaagtagctgggaatgcaggtgtgcaccaccactccagggttttttgttgttgttgttgttgtttttctgtagagacagggtcttgccatgctgccgaggctgctctcaaactcctgggatcaagaaatcctcctgcctcggcctcccaaagtgctgacattacaagcatgagccactgtgcctggctaacttttcatcttttaaagtagtgtcttgcaaagaacaacattttaatgaagtccatttatcaactttttgattcattgtccatgctttttgcataataagaaatctttgcctgcctcaaaattgcaaagctt

SEQ ID NO: 44 - DNA, 363 bp, Homo sapiens
cggccgcccgccttggcccgtctctggagtgctgggcagccgggtctgcgggcccctttacagcacatcgccggccggcccaggtagggcggcctctctccctcgcaagggggcccagctggagctggaggagatgctggtccccaggaagatgtccgtcagccccctggagagctggctcacggcccgctgcttcctgcccagactggataccgggaccgcagggactgtggctccaccgcaatcctaccagtgtccgcccagccagataggggaaggggccgagcagggggatgaaggcgtcgcggatgcgcctcaaattcagtgcaaaaacgtgctgaagatccgccggcggaagatgaa

SEQ ID NO: 45 - DNA, 499 bp, Homo sapiens
atcacaaataggacaatacttgctggtctccaggtaacgaacaatacacgttttacagaaggaatgtagacattctattatggttgtggcatcaatgaagtaccctccacaaagcacacacatcaggtggggatttagctcagtgatcttgattctcgttgttcgatgcatttctgcttgataaaaaatcccggaaagagcagccggcgcgaggcgatcgaagcgggcggaaaagacaatgaaagttaaaagtcgttcagcagaaaatgaatgcgagccaagcggccatcttgaagcgagctgcagacgccgctgtcaatgggcaaccagcgcggccccgagcagccgcggccgccacgctcgtctcatgccgcctccggccggcctcctcctgctccggcgcctcggcctcctccggcgcctcggcctcctcctcctccgcctccgcctcgacctccaacgcctcctcctccggggcctcctcctcctcctcctcggc

SEQ ID NO: 46 - DNA, 761 bp, Homo sapiens
  modified_base (355)..(355): a, c, t, g, unknown or other
aagcttattatctcatcatcagttataattctcttatcttcatctgcaacctctcctctatcttcattagagccattggcagcatcagcagaaggatgagctgcataaaaatcccttcttctcttcatttcatttttgaaaagccctggaactaatttgtatacaatatcttggagagttttatctgaccttatattcagtagtggtctggtcttgtgaacttggacatcacaaataggacaatacttgctggtctccaggtaacgaacaatacacgttttacagaaggaatgtagacattctattatggttgtggcatcaatgaagtaccctccacaaagcacacacatcaggnggggatttagctcagtgatcttgattctcgttgttcgatgcatttctgcttgataaaaaatcccggaaagagcagccggcgcgaggcgatcgaagcgggcggaaaagacaatgaaagttaaaagtcgttcagcagaaaatgaatgcgagccaagcggccatcttgaagcgagctgcagacgccgctgtcaatggncaaccagcgcggccccgagcagccgcggccgccacgctcgtctcatgccgcctccggccggcctcctcctgctccggcgcctcggcctcctccggcgcctcggcctcctcctcctccgcctccgcctcgacctccaacgcctcctcctccgcttgaattcggatccccgagcatcacacctgactggaatacgaacagctccacatncngt

SEQ ID NO: 47 - DNA, 711 bp, Homo sapiens
  modified_base (136)..(136): a, c, t, g, unknown or other
ggaggtcgaggcggaggcggaggaggaggaggccgaggcgccggaggaggccgaggcgccggagcaggaggaggccggccggaggcggcatgagacgagcgtggcggccgcggctgctcggggccgcgctggttgnccattgacagcggcgtctgcagctcgcttcaagatggccgcttggctcgcattcattttctgctgaacgacttttaactttcattgtcttttccgcccgcttcgatcgcctcgcgccggctgctctttccgggattttttatcaagcagaaatgcatcgaacaacgagaatcaagatcactgagctaaatccccncctgatgtgtgtgctttgtggagggtacttcattgatgccacaaccataatagaatgtctacattccttctgtaaaacgtgtattgttcgttacctggagaccagcaagtattgtcctatttgtgatgtccaagttcacaagaccagaccactactgaatataaggtcagataaaactctccaagatattgtatacaaattagttccagggcttttcaaaaatgaaatgaagagaagaagggatttttatgcagctcatccttctgctgatgctgccaatggctctaatgaagatagaggaggacggttgcagatgaagataagagaattataanctgatgatgagataataaggcttgcggccgcactcgagaaacagt

SEQ ID NO: 48 - DNA, 717 bp, Homo sapiens
  modified_base (142)..(142): a, c, t, g, unknown or other
ggcgttggaggtcgaggcggaggcggaggaggaggaggccgaggcgccggaggaggccgaggcgccggagcaggaggaggccggccggaggcggcatgagacgagcgtggcggccgcggctgctcggggccgcgctggttgnccattgacagcggcgtctgcagctcgcttcaagatggccgcttggctcgcattcattttctgctgaacgacttttaactttcattgtcttttccgcccgcttcgatcgcctcgcgccggctgctctttccgggattttttatcaagcagaaatgcatcgaacaacgagaatcaagatcactgagctaaatccccncctgatgtgtgtgctttgtggagggtacttcattgatgccacaaccataatagaatgtctacattccttctgtaaaacgtgtattgttcgttacctggagaccagcaagtattgtcctatttgtgatgtccaagttcacaagaccagaccactactgaatataaggtcagataaaactctccaagatattgtatacaaattagttccagggcttttcaaaaatgaaatgaagagaagaagggatttttatgcagctcatccttctgctgatgctgccaatggctctaatgaagatagaggaggacggttgcagatgaagataagagaattataanctgatgatgagataataaggcttgcggccgcactcgagaaacagt

SEQ ID NO: 49 - DNA, 66 bp, Homo sapiens
cgcgaaatggtgccgcgcatgcgccgcaccagccgcgcgagcattcatcatattaaaccgaccgaa

SEQ ID NO: 50 - DNA, 555 bp, Homo sapiens
ggagtttcacttttgttgcccaggattgagtgcagtgccccgatcttggctcactacaacctctgcctcctgggttcaagcgactctcctgcctcagtgtcctgagtagctgggattacaggcgtctgccaccacgcccggctaattttgtatttttagtagagaacaggtttcactatgttggtcaggctggtcttgaactcctgacctcagcgcatccagaattttagacggggcccccagggtgaggtcttggcaccctccagtagagaagaagggacatgggccatacgtggggtgtcctttctgggagccttgcgtcccttacctgcctagccagggattgcacctcacagcacgcagccagcaggaacggcaccgtgatctgatttcacctgcgggccctgggccctgggggtgttgacaattgggcatatcacagtgtgagctagtcccgtctcggggtttggaggctccacgtggccgtggtacaggagcaggcagttccatcctctggcctggatcaggctctgcacacggaggcctgtgggccag

SEQ ID NO: 51 - DNA, 1278 bp, Homo sapiens
ccattggcctgtagattcacctcccctgggcagggccccaggacccaggataatatctgtgcctcctgcccagaaccctccaagcagacacaatggtaagaatggtgcctgtcctgctgtctctgctgctgcttctgggtcctgctgtcccccaggagaaccaagatggtcgttactctctgacctatatctacactgggctgtccaagcatgttgaagacgtccccgcgtttcaggcccttggctcactcaatgacctccagttctttagatacaacagtaaagacaggaagtctcagcccatgggactctggagacaggtggaaggaatggaggattggaagcaggacagccaacttcagaaggccagggaggacatctttatggagaccctgaaagacatcgtggagtattacaacgacagtaacgggtctcacgtattgcagggaaggtttggttgtgagatcgagaataacagaagcagcggagcattctggaaatattactatgatggaaaggactacattgaattcaacaaagaaatcccagcctgggtccccttcgacccagcagcccagataaccaagcagaagtgggaggcagaaccagtctacgtgcagcgggccaaggcttacctggaggaggagtgccctgcgactctgcggaaatacctgaaatacagcaaaaatatcctggaccggcaagatcctccctctgtggtggtcaccagccaccaggccccaggagaaaagaagaaactgaagtgcctggcctacgacttctacccagggaaaattgatgtgcactggactcgggccggcgaggtgcaggagcctgagttacggggagatgttcttcacaatggaaatggcacttaccagtcctgggtggtggtggcagtgcccccgcaggacacagccccctactcctgccacgtgcagcacagcagcctggcccagcccctcgtggtgccctgggaggccagctaggaagcaagggttggaggcaatgtgggatctcagacccagtagctgcccttcctgcctgatgtgggagctgaaccacagaaatcacagtcaatggatccacaaggcctgaggagcagtgtggggggacagacaggaggtggatttggagaccgaagactgggatgcctgtcttgagtagacttggacccaaaaaatcatctcaccttgagcccacccccaccccattgtctaatctgtagaagctaataaataatcatccctccttgcctagcataaaaaaaaaaaaaaa

SEQ ID NO: 52 - DNA, 9911 bp, Homo sapiens
gcgatcgccatgcagaaggagctgggcattgtgccttcctgccctggcatgaagagccccaggccccacctcctgctaccattgctgctgctgctgctgctgctgctgggggctggggtgccaggtgcctggggtcaggctgggagcctggacttgcagattgatgaggagcagccagcgggtacactgattggcgacatcagtgcggggcttccggcaggcacggcagctcctctcatgtacttcatctctgcccaagagggcagcggcgtgggcacagacctggccattgacgaacacagtggggtcgtccgtacagcccgtgtcttggaccgtgagcagcgggaccgctaccgcttcactgcagtcactcctgatggtgccaccgtagaagttacagtgcgagtggctgacatcaacgaccatgctccagccttcccacaggctcgggctgccctgcaggtacctgagcatacagcttttggcacccgctacccactggagcctgctcgtgatgcagatgctgggcgtctgggaacccagggctatgcgctatctggtgatggggctggagagaccttccggctggagacacgccccggtccagatgggactccagtacctgagctggtagttactggggaactggaccgagagaaccgctcacactatatgctacagctggaggcctatgatggtggttcacccccccggagggcccaggccctgctggacgtgacactgctggacatcaatgaccatgccccggctttcaatcagagccgctaccatgctgtggtgtctgagagcctggcccctggcagtcctgtcttgcaggtgttcgcatctgatgccgatgctggtgtcaatggggctgtgacttacgagatcaaccggaggcagagcgagggtgatggacccttctccatcgacgcacacacggggctgctgcagttagagcggccactggactttgagcagcggcgggtccatgaactggtggtgcaagcacgagatggtggggctcaccctgagctgggctcggcctttgtgactgtgcatgtgcgagatgccaatgacaatcagccctccatgactgtcatctttctcagtgcagatggctccccccaagtgtctgaggccgccccacctggacagctcgttgctcgcatctctgtgtcagacccagatgatggtgactttgcccatgtcaatgtgtccctggaaggtggagagggccactttgccctaagcacccaagacagcgtcatctatctggtgtgtgtggctcggcggctggatcgagaggagagggatgcctataacttgagggttacagccacagactcaggctcacctccactgcgggctgaggctgcctttgtgctgcacgtcactgatgtcaacgacaatgcacctgcctttgaccgccagctctaccgacctgagcccctgcctgaggttgcgctgcctggcagctttgtagtgcgggtgactgctcgggatcctgaccaaggcaccaatggtcaggtcacttatagcctagcccctggcgcccacacccactggttctccattgaccccacctcaggcattatcactacggctgcctcactggactatgagttggaacctcagccacagctgattgtggtggccacagatggtggcctgccccctctagcctcctctgccacagttagcgtggccctgcaagatgtgaatgataatgagccccaattccagaggactttctacaatgcctcactgcctgagggcacccagcctggaacttgcttcctgcaggtgacagccacagacgcggatagtggcccatttggcctcctctcctattccttgggtgctggacttgggtcctccggatctcccccattccgcattgatgcccatagcggtgatgtgtgcacaacccggaccctggaccgtgaccaggggccctcaagctttgacttcacagtgacagctgtggatgggggaggcctcaagtccatggtatatgtgaaggtgtttctgtcagacgagaatgacaaccctcctcagttttatccacgggagtatgctgccagtataagtgcccagagtccaccaggcacagctgtgctgaggttgcgtgcccatgaccctgaccagggatcccatgggcgactctcctaccatatcctggctggcaacagccccccactttttaccttggatgagcaatcagggctgttgacagtagcctggcccttggccagacgggccaattctgtggtgcagctggagatcggggctgaggacggaggtggcctacaggcagaacccagtgcccgagtggacatcagcattgtgcctggaacccccacaccacccatatttgagcaactacagtatgttttttctgtgccagaggatgtggcaccaggcaccagtgtgggcatagtccaggcacacaacccaccaggtcgcttggcacctgtgaccctttccctatcaggtggggatccccgaggactcttctccctagatgcggtatcaggactgttgcaaacacttcgccctctggaccgggagctactgggaccagtgttggagctggaggtgcgagcaggcagtggagtgcccccagctttcgctgtagctcgggtgcgtgtgctgctggatgatgtgaatgacaactcccctgcctttcctgcacctgaagacacggtattgctaccaccaaacactgccccagggactcccatctatacactgcgggctcttgaccccgactcaggtgttaacagtcgagtcacctttaccctgcttgctgggggtggtggagccttcaccgtggaccccaccacaggccatgtacggcttatgaggcctctggggccctcaggagggccagcccatgagctggagctggaggcccgggatgggggctccccaccacgcaccagccactttcgactacgggtggtggtacaggatgtgggaacccgtgggctggctccccgattcaacagccctacctaccgtgtggacctgccctcaggcaccactgctggaactcaggtcctgcaagtgcaggcccaagcaccagatgggggccctatcacctatcaccttgcagcagagggagcaagtagcccctttggcctggagccacagagtgggtggctatgggtgcgggcagcactagaccgtgaggcccaggaattgtacatactgaaggtaatggcagtgtctgggtccaaagctgagttggggcagcagacaggcacagccaccgtgagggtcagcatcctcaaccagaatgaacacagtccccgcttgtctgaggatcccaccttcctggctgtggctgagaaccagcccccagggaccagcgtgggccgagtctttgccactgaccgagactcaggacccaatggacgtctgacctacagcctgcaacagctgtctgaagacagcaaggccttccgcatccacccccagactggagaagtgaccacactccaaaccctggaccgtgagcagcagagcagctatcagctcctggtgcaggtgcaggatggagggagcccaccccgcagcaccacaggcactgtgcatgttgcagtgcttgacctcaacgacaacagccccacgttcctgcaggcttcaggagctgctggtgggggcctccctatacaggtaccagaccgcgtgcctccaggaacactggtgacgactctgcaggcgaaggatccagatgagggggagaatgggaccatcttgtacacgctaactggtcctggctcagagcttttctctctgcaccctcactcaggggagctgctcactgcagctcccctgatccgagcagagcggccccactatgtgctgacactgagtgctcatgaccaaggcagccctcctcgaagtgccagcctccagctgctggtgcaggtgcttccctcagctcgcttggccgagccgcccccagatctcgcagagcgggacccagcggcaccagtgcctgtcgtgctgacggtgacagcagctgagggactgcggcccggctctctgttgggctcggtggcagcgccagagcccgcgggtgtgggtgcactcacctacacactggtgggcggtgccgatcccgagggcaccttcgcgctggatgcggcctcagggcgcttgtacctggcgcggcccctggacttcgaagctggcccgccgtggcgcgcgctcacggtacgcgctgaggggccgggaggcgcgggcgcgcggctgctgcgagtgcaggtgcaagtgcaggacgagaatgagcatgcgcccgcctttgcgcgcgacccgctggcgctggcgctgccagagaacccggagcccggcgcagcgctgtacactttccgcgcgtcggacgccgacggccccggccccaatagcgacgtgcgctaccgcctgctgcgccaggagccgcccgtgccggcgcttcgcctggacgcgcgcaccggggcgctcagcgctccgcgcggcctggaccgagagaccactcccgcgctgctgctgctggtggaagccaccgaccggcccgccaacgccagccgccgtcgtgcagcgcgcgtttcagcgcgcgtcttcgtcacggatgagaatgacaacgcgcctgtcttcgcctcgccgtcacgcgtgcgcctcccagaggaccagccgcctgggcccgcggccctgcacgtggtagcccgggacccggatctgggcgaggctgcacgcgtgtcctatcggctggcatctggcggggacggccacttccggctgcactcaagcactggagcgctgtccgtggtgcggccgttggaccgcgaacaacgagctgagcacgtactgacagtggtggcctcagaccacggctccccgccgcgctcggccacgcaggtcctgaccgtcagtgtcgctgacgtcaacgacgaggcgcctactttccagcagcaggagtacagcgtcctcttgcgtgagaacaaccctcctggcacatctctgctcaccctgcgagcaaccgaccccgacgtgggggccaacgggcaagtgacttatggaggcgtctctagcgaaagcttttctctggatcctgacactggtgttctcacgactcttcgggccctggatcgagaggaacaggaggagatcaacctgacagtgtatgcccaggacaggggctcacctcctcagttaacgcatgtcactgttcgagtggctgtggaggatgagaatgaccatgcaccaacctttgggagtgcccatctctctctggaggtgcctgagggccaggacccccagacccttaccatgcttcgggcctctgatccagatgtgggagccaatgggcagttgcagtaccgcatcctagatggggacccatcaggagcctttgtcctagaccttgcttctggagagtttggcaccatgcggccactagacagagaagtggagccagctttccagctgaggatagaggcccgggatggaggccagccagctctcagtgccacgctgcttttgacagtgacagtgctggatgccaatgaccatgctccagcctttcctgtgcctgcctactcggtggaggtgccggaggatgtgcctgcagggaccctgctgctgcagctacaggctcatgaccctgatgctggagctaatggccatgtgacctactacctgggcgccggtacagcaggagccttcctgctggagcccagctctggagaactgcgcacagctgcagccttggacagagaacagtgtcccagctacaccttttctgtgagtgcagtggatggtgcagctgctgggcccctaagcaccacagtgtctgtcaccatcacggtgcgcgatgtcaatgaccatgcacccaccttccccaccagtcctctgcgcctacgtctgccccgcccaggccccagcttcagtaccccaaccctggctctggccacactgagagctgaagatcgtgatgctggtgccaatgcttccattctgtaccggctggcaggcacaccacctcctggcactactgtggactcttacactggtgaaatccgcgtggcccgctctcctgtagctctaggcccccgagatcgtgtcctcttcattgtggccactgatcttggccgtccagctcgctctgccactggtgtgatcattgttggactgcagggggaagctgagcgtggaccccgctttccccgggctagcagtgaggctacgattcgtgagaatgcgcccccagggactcctattgtctcccccagggccgtccatgcaggaggcacaaatggacccatcacctacagcattctcagtgggaatgagaaagggacattctccatccagcctagtacaggtgccatcacagttcgctcagcagaggggctagacttcgaggtgagtccacggctgcgactggtgctgcaggcagagagtggaggagcctttgccttcactgtgctgaccctgaccctgcaagatgccaacgacaatgctccccgtttcctgcggccccattatgtggccttccttcctgagtcccggcccttggaggggcccctgctgcaggtggaggcggatgacctggatcaaggctctggaggacagatttcctacagtctggctgcatcccagccggcacgtggattgttccacgtagacccaaccacaggcactatcactaccacagccatcctggaccgtgagatctgggctgaaacacggttggtgctgatggccacagacagagggagcccagccctggtgggctcagctaccttgacggtgatggtcatcgacaccaatgacaatcgccccaccatcccccaaccctgggagctccgagtgtcagaagatgcgttattgggctcagagattgcacaggtaacagggaatgatgtggactcaggacccgtgctgtggtatgtgctaagcccatctgggccccaggatcccttcagtgttggccgctatggaggccgtgtctccctcacggggcccctggactttgagcagtgtgaccgctaccagctgcagctgctggcacatgatgggcctcatgagggccgtgccaacctcacagtgcttgtggaggatgtcaatgacaatgcacctgccttctcacagagcctctaccaggtaatgctgcttgagcacacacccccaggcagtgccattctctccgtctctgccactgatcgggactcaggtgccaacggtcacatttcctaccacctggcttcccctgccgatggcttcagtgttgaccccaacaatgggaccctgttcacaatagtgggaacagtggccttgggccatgacgggtcaggagcagtggatgtggtgctggaagcacgagaccacggggctccaggccgggcagcacgagccacagtgcacgtgcagctgcaggaccagaacgaccacgccccgagcttcacattgtcacactaccgtgtggctgtgactgaagacctgccccctggctccactctgctcaccctggaggctacagatgctgatggaagccgcagccatgccgctgtggactacagcatcatcagtggcaactggggccgagtcttccagctggaacccaggctggctgaggctggggagagtgctggaccaggcccccgggcactgggctgcctggtgttgcttgaacctctagactttgaaagcctgacacagtacaatctaacagtggctgcagctgaccgtgggcagccaccccaaagctcagtcgtgccagtcactgtcactgtactagatgtcaatgacaacccacctgtctttacccgagcatcctaccgtgtgacagtacctgaggacacacctgttggagctgagctgctgcatgtagaggcctctgacgctgaccctggccctcatggcctcgtgcgtttcactgtcagctcaggcgacccatcagggctctttgagctggatgagagctcaggcaccttgcgactggcccatgccctggactgtgagacccaggctcgacatcagcttgtagtacaggctgctgaccctgctggtgcacactttgctttggcaccagtgacaattgaggtccaggatgtgaatgatcatggcccagccttcccactgaacttactcagcaccagcgtggccgagaatcagcctccaggcactctcgtgaccactctgcatgcaatcgacggggatgctggggcttttgggaggctccgttacagcctgttggaggctgggccaggacctgagggccgtgaggcatttgcactgaacagctcaacaggggagttgcgtgcgcgagtgccctttgactatgagcacacagaaagcttccggctgctggtgggtgctgctgatgctgggaatctctcagcctctgtcactgtgtcggtgctagtgactggagaggatgagtatgaccctgtatttctggcaccagctttccacttccaagtgcccgaaggtgcccggcgtggccacagcttgggtcacgtgcaggccacagatgaggatgggggtgccgatggcctggttctgtattcccttgccacctcttccccctattttggtattaaccagactacaggagccctgtacctgcgggtggacagtcgggcaccaggcagcggaacagccacctctgggggtgggggccggacccggcgggaagcaccacgggagctgaggctggaggtgatagcacgggggcctctgcctggttcccggagtgccacagtgcctgtgaccgtggatatcacccacaccgcactgggcctggcacctgacctcaacctgctattagtaggggccgtggcagcctccttgggagttgtggtggtgcttgcactggcagccctggtcctaggacttgttcgggcccgtagccgcaaggctgaggcagcccctggcccaatgtcacaggcagcacccctagccagtgactcactgcagaaactgggccgggagccacctagtccaccaccctctgagcacctctatcaccagactcttcccagctatggtgggccaggagctggaggaccctacccccgtggtggctccttggacccttcacattcaagtggccgaggatcagcagaggctgcagaggatgatgagatccgcatgatcaatgagttcccccgtgtggccagtgtggcctcctctctggctgcccgtggccctgactcaggcatccagcaggatgcagatggtctgagtgacacatcctgcgaaccacctgcccctgacacctggtataagggccgaaaggcagggctgctgctgccaggtgcaggagccactctctacagagaggaggggcccccagccactgccacagccttcctggggggctgtggcctgagccctgcacccactggggactatggcttcccagcagatggcaagccatgtgtggcaggtgcgctgacagccattgtggccggcgaggaggagctccgtggcagctataactgggactacctgctgagctggtgccctcagttccaaccactggccagtgtcttcacagagatcgctcggctcaaggatgaagctcggccatgtcccccagctccccgtatcgacccaccacccctcatcactgccgtggcccacccaggagccaagtctgtgccccccaagccagcaaacacagctgcagcccgggccatcttcccaccagcttctcaccgctcccccatcagccatgaaggctccctgtcctcagctgccatgtcccccagcttctcaccctctctgtctcctctggctgctcgctcacccgttgtctcaccatttggggtggcccagggtccctcagcctcagcactcagcgcagagtctggcctggagccacctgatgacacggagctgcacatcgtttaaac

SEQ ID NO: 53 - DNA, 5740 bp, Homo sapiens
cgccgccgccgcacgccgcctgcctcctgcacgccgccgccgcgcctagcgcccgggcccgcgacaccgcccgctaagcgccgggccgagttcacgcagccgcggtctggcggctccgcggcggcggcgggtgcgggcggcctggccggtgccggttaaagggacgagttgcaaacacttcaggaagtgacaagtcgatttcctcctccccgggagtcgctcgtacaaagcgctcggcgccggcaggcgagcgtgcgcgcggcggacgcgcggcgggcaccccggacgacttggcgagcgctggcggtgacggcgcggggtccgcgcccggagcgccccgccgcgcacaggagttgaccacatttggccatttcccagaagggccccaccccaagggtgagtggccaatggggagctgtttctgctgacatcaattccccaggaggtactcaccccaagtctgcccaagtgaagatggctgatacccaccctgggatggagcccagcgcctgaggcccttatcatggtgatggtcctaagtgaaagcctcagcacccggggagctgactccattgcatgtgggaccttcagccgtgaactgcacacgccaaagaagatgagtcaaggacctacacttttctcttgtggaattatggaaaatgacagatggcgagacctggacaggaaatgccctcttcagattgaccaaccgagcaccagcatctgggaatgcctgcctgaaaaggacagctcactatggcaccgggaggcagtgaccgcctgcgctgtgaccagtctgatcaaagacctcagcatcagcgaccacaacgggaacccctcagcaccccctagcaagcgccagtgccgctcactgtccttctccgatgagatgtccagttgccggacatcatggaggcccttgggctccaaagtctggactcccgtggaaaagagacgctgctacagcgggggcagcgtccagcgctattccaacggcttcagcaccatgcagaggagttccagcttcagcctcccttcccgggccaacgtgctctcctcaccctgcgaccaggcaggactccaccaccgatttggagggcagccctgccaaggggtgccaggctcagccccgtgtggacaggcaggtgacacctggagccctgacctgcaccccgtgggaggaggccggctggacctgcagcggtccctctcttgctcacatgagcagttttcctttgtggaatactgtcctccctcagccaacagcacacctgcctcaacaccagagctggcgagacgctccagcggcctttcccgcagccgctcccagccgtgtgtccttaacgacaagaaggtcggtgttaaaaggcggcgccctgaagaagtgcaagagcagaggccttctctagaccttgccaagatggcacagaactgtcagaccttcagcagcctcagctgcctgagcgcagggacagaggactgcggtccccagagccccttcgcccgccacgtcagcaacaccagggcctggaccgccctgctctcagcctccggcccagggggcaggacccccgctgggaccccggtccctgagcctcttcccccttccttcgacgaccacctcgcctgccaggaggacctgtcctgtgaggagtcagacagctgcgccctggacgaggattgtggcaggagagcggagccggctgcagcctggcgggaccgcggggcccctgggaacagcctctgctccctggacggcgagttggacattgagcagatagagaagaactgagggggtgtgggcccaggcagggctggggtgtgctggcatcgacagcccccactctgggcactaggtgggcccttgaaggggagcccaactcgtgggcctgatgaaagcttcctgagtggtgtcgggtcccagagagggagcccacctgctgcctgggggagagcctggcctggccgcgtcatacagcgggtgtgtcagcctctcaccggctccccgagcgtggcagccaccaggtccacagaactactgcagcccagaggacagctttgaagtttgcgtcttttctgcctctttccctgtgggatgttgggcagtctctgttgtccccggcagagctgggcaccgctctgtatccccctggtggtgggggctgtcagggagggcctggggtgggggccaggggccatctgctatgtcagggcccttcttggcctcactcaggttcacttctggggagtcggccccgcagcttctttcactcagttttactccgtgccttctctcccaggtctccctgcttcaggcttgggaaggttcgggagatgcttccttctgtaacaccagaaccatttggccttaattccaatgtgagagacagaatccctggggtgctggactggccctccagagggtaagccatgtccggagtctcgggcccaaggaacgatttggagggtgcttgttagggcctcccgtgttgggtagaaatttggtggatctgttggctgaaaagacggacttgcttgcctctcctacagcatggagaggctgaccccatggctctgccaccgttggggcagggttagcagatggcagcccttctctgtggctgacaggtcactgagtgataagcatggttggttccggtgagtgtagggatggcacgataccagggcagcctcttgaaaacggcctcgggagacgggagctgcgagcaggtgggcagatgagggccctatgcgcactcaggggtgaagggcgtccgctggccactctgcaggggcccctgcaggattccaggcacctcccgtttgtccttgaggactgctggctgtaaccagggcacatcacccacctcaagacaagcccacgcccttgtcagcttagggggagcccagtcctgagggctgcatctctgttgtaggcccagccaccggcacaaagctggattcatgctccctgcccctaccccaccctggctcctcaccctggggcatccgaggagcctagccccctgagggtttgctctcctctcaaggtttgtagctcctctccggctgccttgcagacaccaccacatgggctctgctctatgggaatctggcttttagcgaatgtggcgtcttctgcaaacaatagcaattgggctggcttaggagcaagtggctcattttcccataaggctaaaaataactggtgcgctcccttgtgttggctgacacgcgcgttcaaagcacttttgtagtcactttgcttttgctcgtcttcatggacgagtgaacgcctcgcttctgcaggttgagtccagatgcttctcaccttctttctcctcaagaaagatgctttttgggaaacgttgtttaaatcttatttttttactacatcaaaaggatggtggttcaagttcccaatatgtgggtggcacttcttaaaaatcagctttaaggagctggcagaaagcccccagccccacagccctgagagatggtgttgctagctcaggtggctgacacatggggtatgccgggcactgggcaggtcccagagccggggaaccagctcacctctggttgctgtagctcctgccggaggcatgtctacttgtgatcccggacagccgaacccaagagctggtggctctgagcagacagagacatcttggcctgtccctgcctgggggtcatggagaccatgtcttcttagagcaaatgtggaggcggccagggcagttgttgggtgaatgtggagagcacatggccatgtcttgcccccggagtaccactgggcgtggggggtcctggcaccacatgcccggtgtggccgagggcacacagcctctatagcaggccttcctgtggaaggcagaggcagtgagggaggtggacggtgccagctgaggctgaggcatgcagcagcccccagctacctttgcttagggctggggtgggaggcacatggtgacaggtatatgtcgtgggactggggtgtgggtgacctgccctcaaaccttgcctgccacctccccattcaggcctggtggcaggaagggacaagctgtggagctggctgagtcacagccacctccccacctccccgcaagctggtcccatcgaccagcaagcccagccccagggcgcttagggagaaatgacccagcctcctcagaccccgcctgcctgtcctgtgcccaccacgcagcagtcaggggagaaaatggtggctatcccttctgcttagagaaagaaatggcctttagctggtttcatgtttgtgttttgactggagggagtagaccctatctataaggtgccaccccatcatccaagctgccacactgcccggagcagcctgttcctgcactccaccctgctggccccaggacttctgatctcagtcctctgggagggaggttcgcctaggaggtgccccccacattggtgtccccatgggcagcaggcagacagctcacccccaccagcatgatggccccagctgggggcagtggcaggagccttacttttgtcacagccttgcccacaaaccctgcctctgaggggagactgaggaagggcagagccagaagcaagccgtgccaggccatctgcctgctcatggggtcctaaagcgcgggctaagcctgcaggaaagccggggcggtggggggggcttagtgccacatgcaccccactcattccaaagccaccaaactgccaggggctgccgtccacccgtggggcccaggggctggggccacagccttgccattttcgttgccataccctcttgccttactcgcggtggaggccggatttgcacgggcagacgtgcacctgggcccgtggggagcttgttctgaccagacgtacagattttcattctcagaaagccttacttttcaaccaaatttttgtagccagttttgtgaatttgtacactgaaagaaaatttaaataaaggggaagtccacattaaaaagaaaacaaaacaaaccctaactaacttccaaatgggtctcctggtgcgggggcgtgagtggccgtgccctgggtgtgctgcctgtctgagcaagcttccctagctgtggaaccccgggccccctgctgcgggctctgccttggtgtcatgcctgctgcacccccgtttccactgacgtgccgtctgtggctatgggggtggtcactggaatgacggtcactccagacgtcagccggcagggatgcagcaggctggccgcgcaccggggctcgggcaccctctggccccacactggcaatgatgccacaccttgccatgtccacgctgttggtcaaacccctctgtcatgcctctttaaagagaaaagaagagaaagattttttttttttttaatggcagaccgaagtggagatcttgtagcctagataggatagtctgaccttctagcatagtctttttggcaaatgatttgtgttttcagtgtgtggggaagctgtcctgggggctggggcgacagatagcacataggctgtttctggggctgcaggggcttccctgagctggatgttgtgggtgttgccgtgcttcaggaagtgtggcgaccagaaagcgtagacccggggcccagggtctgcccgcccctgcagcctggcctccccgcacaggctgtggcttgcactccagccgctctagtctctcaggaatttgcttgttacttgtactgtgtaaataaagcttcctggttcaataccc

SEQ ID NO: 54 - DNA, 5602 bp, Homo sapiens
atggagccctccagagcgcttctcggctgcctagcgagcgccgccgctgccgccccgccgggggaggatggagcaggggccggggccgaggaggaggaggaggaggaggaggaggcggcggcggcggtgggccccggggagctgggctgcgacgcgccgctgccctactggacggccgtgttcgagtacgaggcggcgggcgaggacgagctgaccctgcggctgggcgacgtggtggaggtgctgtccaaggactcgcaggtgtccggcgacgagggctggtggaccgggcagctgaaccagcgggtgggcatcttccccagcaactacgtgaccccgcgcagcgccttctccagccgctgccagcccggcggcgaggaccccagttgctacccgcccattcagttgttagaaattgattttgcggagctcaccttggaagagattattggcatcgggggctttgggaaggtctatcgtgctttctggataggggatgaggttgctgtgaaagcagctcgccacgaccctgatgaggacatcagccagaccatagagaatgttcgccaagaggccaagctcttcgccatgctgaagcaccccaacatcattgccctaagaggggtatgtctgaaggagcccaacctctgcttggtcatggagtttgctcgtggaggacctttgaatagagtgttatctgggaaaaggattcccccagacatcctggtgaattgggctgtgcagattgccagagggatgaactacttacatgatgaggcaattgttcccatcatccaccgcgaccttaagtccagcaacatattgatcctccagaaggtggagaatggagacctgagcaacaagattctgaagatcactgattttggcctggctcgggaatggcaccgaaccaccaagatgagtgcggcagggacgtatgcttggatggcacccgaagtcatccgggcctccatgttttccaaaggcagtgatgtgtggagctatggggtgctactttgggagttgctgactggtgaggtgccctttcgaggcattgatggcttagcagtcgcttatggagtggccatgaacaaactcgcccttcctattccttctacgtgcccagaaccttttgccaaactcatggaagactgctggaatcctgatccccactcacgaccatctttcacgaatatcctggaccagctaaccaccatagaggagtctggtttctttgaaatgcccaaggactccttccactgcctgcaggacaactggaaacacgagattcaggagatgtttgaccaactcagggccaaagaaaaggaacttcgcacctgggaggaggagctgacgcgggctgcactgcagcagaagaaccaggaggaactgctgcggcgtcgggagcaggagctggccgagcgggagattgacatcctggaacgggagctcaacatcatcatccaccagctgtgccaggagaagccccgggtgaagaaacgcaagggcaagttcaggaagagccggctgaagctcaaggatggcaaccgcatcagcctcccttctgatttccagcacaagttcacggtgcaggcctcccctaccatggataaaaggaagagtcttatcaacagccgctccagtcctcctgcaagccccaccatcattcctcgccttcgagccatccagttgacaccaggtgaaagcagcaaaacctggggcaggagctcagtcgtcccaaaggaggaaggggaggaggaggagaagagggccccaaagaagaagggacggacgtgggggccagggacgcttggtcagaaggagcttgcctcgggagatgaaggatcccctcagagacgtgagaaagctaatggtttaagtaccccatcagaatctccacatttccacttgggcctcaagtccctggtagatggatataagcagtggtcgtccagtgcccccaacctggtgaagggcccaaggagtagcccggccctgccagggttcaccagccttatggagatggccttgctggcagccagttgggtggtgcccatcgacattgaagaggatgaggacagtgaaggcccagggagtggagagagtcgcctacagcattcacccagccagtcctacctctgtatcccattccctcgtggagaggatggcgatggcccctccagtgatggaatccatgaggagcccaccccagtcaactcggccacgagtacccctcagctgacgccaaccaacagcctcaagcggggcggtgcccaccaccgccgctgcgaggtggctctgctcggctgtggggctgttctggcagccacaggcctagggtttgacttgctggaagctggcaagtgccagctgcttcccctggaggagcctgagccaccagcccgggaggagaagaaaagacgggagggtctttttcagaggtccagccgtcctcgtcggagcaccagccccccatcccgaaagcttttcaagaaggaggagcccatgctgttgctaggagacccctctgcctccctgacgctgctctccctctcctccatctccgagtgcaactccacacgctccctgctgcgctccgacagcgatgaaattgtcgtgtatgagatgccagtcagcccagtcgaggcccctcccctgagtccatgtacccacaaccccctggtcaatgtccgagtagagcgcttcaaacgagatcctaaccaatctctgactcccacccatgtcaccctcaccaccccctcgcagcccagcagtcaccggcggactccttctgatggggcccttaagccagagactctcctagccagcaggagcccctccagcaatgggttgagccccagtcctggagcaggaatgttgaaaacccccagtcccagccgagacccaggtgaattcccccgtctccctgaccccaatgtggtcttccccccaaccccaaggcgctggaacactcagcaggactctaccttggagagacccaagactctggagtttctgcctcggccgcgtccttctgccaaccggcaacggctggacccttggtggtttgtgtcccccagccatgcccgcagcacctccccagccaacagctccagcacagagacgcccagcaacctggactcctgctttgctagcagtagcagcactgtagaggagcggcctggacttccagccctgctcccgttccaggcagggccgctgcccccgactgagcggacgctcctggacctggatgcagaggggcagagtcaggacagcaccgtgccgctgtgcagagcggaactgaacacacacaggcctgccccttatgagatccagcaggagttctggtcttagcacgaaaaggattggggcgggcaagggggacagccagcggagatgaggggagctggcgggcacagccctttctcagggttggaccccctgagatccagccctacttcttgcactgataatgcactttgaagatggaagggatggaaacagggccacttcagagggtctcctgccctgcagggcctttctacccgtgtccactggaggggctgtggccatcagctctggctgtgtaggggaggaaggggtgcatgcatgtcccccaccctccacagtcttccttgcctttagagtgaccctgcagagtcactcagccaaatctgtctgctgctccctctcctcagccagttgggtgtgcgcagagctgtcatagggtccctttgtcagccccgagttcagcttcccaaacaccagtgttggatattctgtgattgattttggtcctcctccgctgtcccccaacacccaggaatgggaatctggcttggttcgagataggagcttttctgtgtcctaagccctttcatgctagcaggaagactgaaagcaaggtggcccagtgtggggtcatagggcttgatagacctggcactgcctatctgcacttccaggtgccccacctatttatctgagcccacaggtggaaaggggaactgcctcagtgagaacggggggacggggatgttaggaaaaatacagtaaagttgcaatgaagaggttcatgaagtatgtccttgttctttttggaaactctcggcaaagggcaaaccagcaagtattgagggtacccatctagctacttggggtcaggacctcgtcagaccaggttcggatacaatcatctgctcatcccaggaatagtttcttgggggactcactcactggtgccagttctaagtcagagacaaaattccactgtctgttccttttgctgtctgaactttatgtgttactcccttcctttggtcttcactctaatccctggagtttgtgggcttttggttatgtttggttagtagatatcaccgcaatgccctagaacagctatgaagcagaataccatatggccacctggacattgggacttgggaattcactctcaactgggccatccatgttgtgatgcccttgaagtaaaatggagccagcaggagtaccttctgtaaatgcatgtggcaaagtgctatttatagggtgcccagggagccgctgatgtacaataaccttgaggtcccccatactgaaaactgaccaaggcctgtgcacaggtagcccctcatgctgggctctggaccatgagctgagtaggaaggatagcagaggccaaccctgaccttcctggaagttgtttccttaacttgaatgttgagcttcctctaaagctttctcgtgtatgtcttctccatgccactactctgaggcctcctgtgttatgtgtgaacagttgtctttatgtgggaatgacgacttgattgggagtagagtctcaaggtcattcccctcttccctcaagactctctgaatgctgctccactgtcttttgtcttggaggtcactcagcaggttccttgcatttgctgcctggatgtgcagctggcaacagtgatgaattggtcactgctctttctctataactgggatagatgtcctgccttggggtcactaaaggggtgaccttgttccttgctttatgagcccattagcactttggttcaaggggcccaccaagtcttggacgggaaggcgctactggttttattgcccaaggttttgttattgcttctcttctgtgtccttctctttgttcagtgaagccaatatgtaagatactgtttttgtccccattcccctactcctgagctaggaggaaaaaatgtgaatcttaccagcagttccagccaaccaagtgattcttcttcattcttgatggggagaagtacatacaaagtttgttctgacagggcgcggtggctcacgcctgtaatcccagcgctttgggaggcagaggcaggtggatcacctgaggtcgggagttcgagaccagcctgaccaacatggagatatcctgtctctactaaaaatacaaaaaaattagccaggcatggtggcacgtgcctgtaatcccagctactcgcaaggctgaggcaggagaatcgcttgaacctgggaggcggaggttgcagtgagccaagattgcgccattgcactccagcctgggcaacaagagagaaactctgtctcaaaa

SEQ ID NO: 55 - DNA, 1980 bp, Homo sapiens
ttcgattttggtgctgtgaaaagaatagaaaagaaaaagaaaatgaagaggtaagctcatagcagattctctttgtatggatttaagggaaggacattatccacaacagaaaactgaccatttggattttcttgtttgtagaaggtctttaacatttccactgcttcctcagcccgatatccagggatacactgatggaatgagaaagttgagaataaacataggcctatgaaaatgtgtgctgtatcccataaaaacaacatatatatacatgattatgtaaacagatttcagatgttaataaactttggggatattagtaacatgggtaaggaggtacacttccaaaagatgtttgatatatcatctttttcattactcccaatcaactgttattaggcatcactcccaatcaactgttattcatccattaactattatagaagttaccagctttgtgatcttgggttaggcacttaaactctccatgccttatttatacaatgctggcaataatagcacttacttcaggggattttgtgaggattaagtgagataatacctgttaaataccaggcacatcataagtgctcattaagcattagttatttttatctgctcctatttactagtggtccattaagcattccatgctatagagctagggttggcaaattatacttggtggaccaaatctgttccatagctgagaactgtgagctaagaatggtttttatatcttaaaagctttgttaaagaaaaaaaaagactaggtgacagagatgtaagcggctcacaaagggtgaaatatttactagttaaccctttgcagaaaaagtttatcaacccttgctacagaggattttaaaaaataaaatacagcttgttctatctttagcatctaactggggaaaagaaatcataacatgtgaaagaataaataagaaattgtgctaacagtaaggagtgttatatgaaatattacctgaagaacatgaaacttgaacttgccttagagatagagaatatttaaagaggctaagcagagcatttcagggaaagggcaagaagaagcctgggttgtgtgtgaggaaatcagctgacagaggaggagactattaaggaagcataaggaaagaaagacaaaaaattggggtaaaaatatgtacggctttgaaagcttgtcagaagagtttggacttaaaaccaagcacccttctgaagtgcatgaagtgacacaatgagcatctggaaggaaggagccagaaagcataggcacagaggacaggaggaccagctactgtgagatgctgttcagaacgaacctcccattctcctgtgtcttcagtctgcccttgcctgggcctccgacacctgcataaaccttcgccataacaaataaccttccatccaccctgtcccgtcaaaggctgacaccctgctcctgccttcactcctcagtggcctcatcttcactggcttgagttcccagcacttcactgagtctgccctctcagaaatccccaggtccctactgaccaaaacacttgcctcctttcagattcctcaactctgcagtcctggaggcaactggccacacctgctctgtctgaccgctcttgcctcccttggcttctcagcattttaccatcctaaccactgccagccagtcccgtcacagctgccccctgcttcctgctgtgttaagtgctggagctccccagaggtccccctccactccactcgcacactcagagccctctcctcttacgtgggatgagagcagtggttctcaaccattgctgctcaggagaaccagttggaactctctggaaacacagcactgttggccccctgccttctgattcagatggtctggggcagggactgagcagagtcaggcacagaagcctccaggtgattctaacgggcagtccgggatgagaactgctgagttacaggcctcgaaggaaactgcac

SEQ ID NO: 56 - DNA, 3939 bp, Homo sapiens
ggtcggcctctgctgcgcctgcgtggtcgggaggggaagtgaggcggtttcctcggcgccttttccggcagcggcggcggcagaactgggaggaggagttggaggccggagggagcccgcgctcggggcggcggctggaggcagcgcaccgagttcccgcgaggatccatgacctgacggggccccggagccgcgctgcctctcgggtgtcctgggtcggtggggagcccagtgctcgcaggccggcgggcgggccggagggctgcagtctccctcgcggtgagaggaaggcggaggagcgggaaccgcggcggcgctcgcgcggcgcctgcggggggaagggcagttccgggccgggccgcgcctcagcagggcggcggctcccagcgcagtctcagggcccgggtggcggcggcgactggagaaatcaagttgtgcggtcggtgatgcccgagtgagcggggggcctgggcctctgcccttaggaggcaactcccacgcaggccgcaaaggcgctctcgcggccgagaggcttcgtttcggtttcgcggcggcggcggcgttgttggctgaggggacccgggacacctgaatgcccccggccccggctcctccgacgcgatggggaaggtgctatccaaaatcttcgggaacaaggaaatgcggatcctcatgttgggcctggacgcggccggcaagacaacaatcctgtacaagttgaagctgggccagtcggtgaccaccattcccactgtgggtttcaacgtggagacggtgacttacaaaaatgtcaagttcaacgtatgggatgtgggcggccaggacaagatccggccgctctggcggcattactacactgggacccaaggtctcatcttcgtagtggactgcgccgaccgcgaccgcatcgatgaggctcgccaggagctgcaccgcattatcaatgaccgggagatgagggacgccataatcctcatcttcgccaacaagcaggacctgcccgatgccatgaaaccccacgagatccaggagaaactgggcctgacccggattcgggacaggaactggtatgtgcagccctcctgtgccacctcaggggacggactctatgaggggctcacatggttaacctctaactacaaatcttaatgagcattctccacccatcccctggaaggagagaaatcaaaaacccattcataggattatcgccaccatcacctctttcaattgccactttctcttcttttgaatttgaactctggagttactgttctacagtttggcggggacggggcttgggggttttctcttttgtttgtttccctttctttttccttttttttttttttttttttttgttggctttgcgttaggatgctctgatctgacatttgacatgaacacaaagttgctagatgctcttgttgacttccagcagatgggatgggggaaacacagcagttcttggtaaagtcctttgtaataatagtttgatttttttatttcgagagaatctttcattttcctatgtatgcttttttccttttttgcccagtttccttatcacttgctgtagatggcttattttgcattcatgcagactatgttgcaagtctgtttcatctagtaaactgaaaattattgcttaatcaaactgccgtttgtcttttatatttaaggccttccccccccttccttatgagttctaacttagtaatttcaaatgtgaccttttatatctaagaccagtatagtaaacttagcccacagtggcaaataatgagtaatattgtaatatgttccagttgcacctcagtatgttaaacaggtaatgtaagaagttctctgaaatgtcagcaagtaagttctgaaacacatcatgcatgagtaggaataaaacccaagttccccataacgtagataacttaatgctgcataaaaatatgaaagtgtaacccatgaaggacactttttctttccactgcaaagttagccactttgctgtttttcctcttttttaaactttgaaaatagactctttccagaaattggagcaataatggtgttaccacacacagattaaataatttgtagatattttaagtgacttttgggcaaaactggaatgtatacttttaccttgtttcaaacacctaagaccagtaatttaaaaattactaaaaggtttactttgttcattaataaaacatttaacaattcaaattatatgcaccttttacctagttgaaaaaaatacacattcctgttttcacattatagcaactgattaagctgaagctgtaagtcattttttatagatgagtgatccgcatctccatcaattagaacactggaaaagatgttttataaaagaggtatttaattttgtttgtaggattaactcatgcaaataataaaaaagatatcctgttggttcaatagtacactgtctcctttaaggaaggaagcgtgatgaatgaatgatgtgtagacttgagggatgactattaaaggggacgtaggatgaagagaaagaacctacagatgacaatgaatgtaaacttatttttcttcatgtgtaagcagtgtgctcgctggtgatatccagatcctaacaagattacttggttagctggttaggaccagtaactggattgcgaccactatgataatattttgaaccaaatgttaatgcttgatgcagaattgtaaagcagcatctggttcctatatagccttaaggattaattttagtgatcctcaaggaattaaatagggaatttcagaaatgtagactgcaaaggcagtatacaggaaaaggtggagtgggttttgtttatgagggtgtctgaaaactaaaattgagcgggatatcatggtatagttggacagtattggtccttcacactttggccatattgtataatggagcttttaccaaagatgtatgagaagtgtaagactataaaaaaatgaactattcaaagtaaaactcttaacaaacattttacttaaagcagatgcaaaagggtattctcatgtaggctcctgttggtgcagagggatttttttgatttcaggatacaactaaagtacgaagttctcagtttcactttagtagaaagagctctagaaatgaggctgataaacacatctaagaacactggttgctttctaaaatttccaaagctccaccataaatgtaatttttagtgtttcaaatgattgcattttaaagtatataaatatgggttatccaatatcaatgctatagtaacatcctgaaacaaaacaagcacaaaggtataaatgcctaaactggaggaaacttgaaaccctcatgttaaatcttaaatgtagtatttctaacttgtgaagacagattggtaggcagccatttttttgtgtcttaaaataactgggggcatagttaaaattttatacatcaagtgattgctattattgaatgttgcaggtgagatgtggttatttttagtttatttgaaatgtttgactggaaaggggggagggggaagcaaatatttgaaatttggaaaaccctaaaccttttggtaagaaattgtaattttcacttaaaattttctttaaggatataagaggtttataattgatgtagttaaattgaacaataaccattggtgactggagcaggtaattatagcctgcagaaaaaattatctaagaattttaaaaataagatcctgaagttgtttaattgcatccatttctgtatttatgtgaatttataaactgcagtaagttttgaatgaggttaatcttgtttaatataagtaaatgagtctgtagactgtgatctccccaaactaaaaagtacagtacttggaattgtgttctttatggttgtagtgttggtaaagcactaatatgcagaaaataaaggaattacacagtgca

SEQ ID NO: 57 - DNA, 5634 bp, Homo sapiens
ttgcgcgctgcagggcaacaccccggcgtccctggaagctgggggagcgggagaaataactttatttggactgagagctggagaatgagaataggacctgagagtatattgggctaaggaggagaggtgtttgagcccagatgagtcatggctggacgacccctccgcataggagatcagctggttctggaagaagattatgatgagacctacattcctagtgagcaagaaattcttgaatttgcccgggagattggtattgatcccatcaaggaaccagaactgatgtggctggcgcgagagggcatcgtggccccactgcctggagagtggaaaccatgccaggacatcacaggtgacatttactatttcaacttcgccaacgggcagtctatgtgggaccatccatgtgacgaacactatcggagcttggtgatccaagagcgggcaaagctgtcaacttctggggccattaagaagaagaaaaaaaaaaaggaaaagaaagacaagaaggacagagacccccccaaaagttcgctggccttgggttcctcattagccccagttcatgttcctcttgggggcctggctcctttacgaggtcttgtggataccccaccctctgctcttcgtggatctcaaagcgtgagcctggggagctcagtggagtctggacgtcagcttggagaactcatgctgccttcacagggtctcaagacctctgcttatacaaagggtctcttgggctccatatatgaggacaagactgctctcagcctcttgggtttaggagaagaaaccaatgaggaggatgaggaggaaagtgacaaccagagtgtccacagctcaagtgagcctcttaggaacctacacctggacattggggcactggggggtgactttgagtatgaggagtctctgagaacaagccagccagaggagaagaaggatgtttctctggattcagatgctgccggtccccctactccctgcaagccctccagcccaggtgcagacagcagtctgagcagtgctgttggcaaagggcgacagggaagtggagcaagacctggtcttccagaaaaagaggaaaatgagaagagtgaacctaagatttgcaggaatctggtgacccccaaggcagaccctacaggcagtgagcctgccaaagcctctgaaaaggaagcaccagaggacacagtagatgcaggagaggagggttccaggagggaagaggcagccaaggagccaaagaagaaggcttctgctctggaagagggcagttcagacgccagccaagaactggaaattagtgaacacatgaaggaaccacagctctcagactccatagcttctgaccccaagtccttccatggcctggacttcggttttcgcagccggatctcggagcacctgctggatgttgatgtgctttccccagtcctgggtggagcttgtcggcaggcccagcaaccactgggaatagaagacaaggatgacagccagtccagccaagatgagctgcagagcaagcagtccaaaggcctggaggagaggttatctcctccacttccacacgaggagcgggcccagagtccccctcgcagcctggccactgaagaagagcctccccagggccccgaggggcagcccgagtggaaggaggcagaggagcttggggaggactctgcagccagcctcagcctgcagctgtccctccagagggagcaggccccaagcccacctgctgcctgtgagaagggcaaggagcagcattcccaggccgaggagctgggccctgggcaggaagaggcagaggatcctgaggagaaggtggcggtcagccccaccccgccagtctctccagaggtgcgatccacagagcctgtggctcccccagagcagctctcagaggctgcactaaaggccatggaagaggcagtggcccaagtactcgagcaagaccagaggcacctgctggaatccaagcaagagaagatgcagcaactgcgggagaagctgtgccaagaggaggaagaggagatcctccggcttcaccagcagaaagagcaatctctcagttccttgagggagcggctgcagaaagccattgaggaggaggaggcccggatgagagaggaggaaagccagaggctatcctggctccgagctcaggtccagtccagcacacaagcagatgaggaccaaatcagggctgagcaagaggcttccctgcagaaactgagagaagagttggagtctcaacagaaggctgagagggccagcttggaacagaaaaataggcaaatgctggagcagctcaaggaagagatagaggcttcggagaagagcgagcaggctgccctgaatgctgcaaaggagaaggctctgcagcagctgagggagcagctggaaggggagaggaaagaagctgtggcaacgctggagaaggagcacagtgctgagctggagcggctctgctcctcattggaggccaagcaccgggaggtggtctccagcctccagaagaagatacaggaagctcaacagaaagaggaggcccagctgcagaagtgccttgggcaagtggagcacagagttcaccagaagtcttatcacgtggctgggtatgagcacgagctcagcagtctcctgcgagagaagcgccaggaagtggaaggggagcatgagaggaggttggacaagatgaaggaggagcaccagcaagtgatggctaaggccagagagcagtatgaagctgaggagaggaagcagcgggctgagcttctggggcacctgaccggagagctggagcgcctgcagagggcccatgaacgagaactggagactgtgaggcaggagcaacacaagcgtcttgaggacttgcggcgccggcacagggagcaggaaaggaagctccaggatttagagttggaccttgaaaccagagctaaagatgtcaaggccagattggctctgctggaggtccaggaggagaccgcccggagggagaagcagcagctgcttgatgtgcagaggcaggttgctctgaagagtgaggaagccacagccacccatcagcagctggaggaggcacagaaggagcacacccacctgttgcagtcaaaccagcagctccgagaaattcttgatgagctgcaggcccgcaagctgaagctggagtcccaagtggatctgctgcaggctcagagccagcaactgcagaaacacttcagcagcctggaggctgaagctcaaaagaagcagcacctgttgagagaagtgacagttgaggaaaataatgcttccccacattttgagccagatctccatattgaggacctgaggaaatcccttggaacaaaccagaccaaagaggtgtcttcttctctctcccagagcaaggaggacttatacttggacagcctgtcctcccacaatgtctggcacctcctctctgctgagggggtagccctccgtagtgccaaggagttccttgtgcagcagacacgctccatgcggaggcggcagacagctctgaaagctgcccagcagcattggcgccatgagctggccagtgcgcaggaggtggccaaagacccaccaggcatcaaggccctggaagatatgcgcaagaacctggagaaggagaccaggcacctggatgagatgaagtcggccatgcggaaaggccacaacctgctgaagaagaaagaggagaagctgaatcagttggagtcctctctttgggaagaggcctcagatgagggcactctgggaggatcccccaccaagaaggcagtaaccttcgacctcagtgacatggacagcctgagcagtgaaagttctgaatctttttccccgcctcaccgtgagtggtggcggcagcagaggatcgactcaaccccgagtctcacctcccgcaagatccacgggcttagccactccctccggcagatcagcagccagctgagcagtgtcctcagcatcctggacagcctcaaccctcagtcgccgccgccgctcctcgcctccatgccagcccagctccctccccgggaccctaagagcacccccacccccacctactatggctccctggccaggttctcagccttatcatctgctacacccacgtccacccaatgggcctgggattcagggcaggggcccaggctcccctcctctgtggctcaaacggtggacgacttcctgttggagaagtggcgcaagtattttccatctggcatcccgctgctcagcaacagccccaccccgctggagagcaggctgggttacatgtctgccagtgagcagctccggctcctacagcactcccattcgcaagtccctgaggcgggcagcaccacctttcagggcataattgaggccaaccggaggtggctggaacgtgtcaagaatgaccccaggttacctctcttctcgtcaacacccaagccaaaagctactttgagcctcctgcagctgggccttgatgagcacaacagagtgaaggtgtatcgcttctgaggccctgagcaggggcttggggcagcccagcctctcctccacccagaccaagtgcctgaggagctgcctgccttcttccatctgagaaagcaccctccttccccctttgacttgcaggagccaccagggaccagggggttgagtggaacagtaaagccacacattctgtgactatataacctatctcaggctaaaatgtgtggactcgtacgagctcttgtcattgacatggcaagctgatggcgtgcggtggctgcggggtatcagggccgggagccctttgggaggaagggaggcgttagaggagctgccttcggaggctcagggagtccctttggagctggttgtttccttggccctgcagcgcactgctcggggctcccaaggaggttgtgtgtatggttcttaattcatcaggacaaagacccccagcatgtgtgtaccctgggacccgatttctctgggcccacatctatctccaatacctcagcctcagatcagaccctttcttttttgtctttcttctcttaatttttaaatgcctcttttcttgagcattccatctctctttttgaccctctcaggactgggcttagctgtccagagccctgccggagggtgctgggggctgtccctctgcaggcactgtgttttcctcaggggctgtcctcagaacacccctcctgctccctggggctcctcagggagccatttcagctggagtctcaggtctcaaaaacaacttctccaggaggccaaaaaaagactgggttggcttctggtcctcatgatggcttttatcctcctgggacactttgggtatattcatgggcattgtttccatctgtcttttctacctgtgccacccctgccctgattccacggctgcctcaggcaggcaggcaaggagctaggccggtgcccggccctggcagcaaggggtctttgtgcagttggagatgctgccgttgtggcagagcgtcctgcagccccgcttccatcagcaggctctggggtgggggctttgcaggggatgctctctgatgtttgttccgttgtttaaataaaatgcacttatttttgtttttttttttgcaaaaaaaa

SEQ ID NO: 58 - DNA, 4005 bp, Homo sapiens
cggcagggttggaaaatgatggaagaggcggaggtggaggcgaccgagtgctgagaggaacctgcggaatcggccgagatggggtctggcgcgcgctttccctcggggacccttcgtgtccggtggttgctgttgcttggcctggtgggcccagtcctcggtgcggcgcggccaggctttcaacagacctcacatctttcttcttatgaaattataactccttggagattaactagagaaagaagagaagcccctaggccctattcaaaacaagtatcttatgttattcaggctgaaggaaaagagcatattattcacttggaaaggaacaaagaccttttgcctgaagattttgtggtttatacttacaacaaggaagggactttaatcactgaccatcccaatatacagaatcattgtcattatcggggctatgtggagggagttcataattcatccattgctcttagcgactgttttggactcagaggattgctgcatttagagaatgcgagttatgggattgaacccctgcagaacagctctcattttgagcacatcatttatcgaatggatgatgtctacaaagagcctctgaaatgtggagtttccaacaaggatatagagaaagaaactgcaaaggatgaagaggaagagcctcccagcatgactcagctacttcgaagaagaagagctgtcttgccacagacccggtatgtggagctgttcattgtcgtagacaaggaaaggtatgacatgatgggaagaaatcagactgctgtgagagaagagatgattctcctggcaaactacttggatagtatgtatattatgttaaatattcgaattgtgctagttggactggagatttggaccaatggaaacctgatcaacatagttgggggtgctggtgatgtgctggggaacttcgtgcagtggcgggaaaagtttcttatcacacgtcggagacatgacagtgcacagctagttctaaagaaaggttttggtggaactgcaggaatggcatttgtgggaacagtgtgttcaaggagccacgcaggcgggattaatgtgtttggacaaatcactgtggagacatttgcttccattgttgctcatgaattgggtcataatcttggaatgaatcacgatgatgggagagattgttcctgtggagcaaagagctgcatcatgaattcaggagcatcgggttccagaaactttagcagttgcagtgcagaggactttgagaagttaactttaaataaaggaggaaactgccttcttaatattccaaagcctgatgaagcctatagtgctccctcctgtggtaataagttggtggacgctggggaagagtgtgactgtggtactccaaaggaatgtgaattggacccttgctgcgaaggaagtacctgtaagcttaaatcatttgctgagtgtgcatatggtgactgttgtaaagactgtcggttccttccaggaggtactttatgccgaggaaaaaccagtgagtgtgatgttccagagtactgcaatggttcttctcagttctgtcagccagatgtttttattcagaatggatatccttgccagaataacaaagcctattgctacaacggcatgtgccagtattatgatgctcaatgtcaagtcatctttggctcaaaagccaaggctgcccccaaagattgtttcattgaagtgaattctaaaggtgacagatttggcaattgtggtttctctggcaatgaatacaagaagtgtgccactgggaatgctttgtgtggaaagcttcagtgtgagaatgtacaagagatacctgtatttggaattgtgcctgctattattcaaacgcctagtcgaggcaccaaatgttggggtgtggatttccagctaggatcagatgttccagatcctgggatggttaacgaaggcacaaaatgtggtgctggaaagatctgtagaaacttccagtgtgtagatgcttctgttctgaattatgactgtgatgttcagaaaaagtgtcatggacatgggaaatgaatactgcattgagggacggacttctggtcttcttcttcctaattgttccccttattgtctgtgctatttttatcttcatcaagagggatcaactgtggagaagctacttcagaaagaagagatcacaaacatatgagtcagatggcaaaaatcaagcaaacccttctagacagccggggagtgttcctcgacatgtttctccagtgacacctcccagagaagttcctatatatgcaaacagatttgcagtaccaacctatgcagccaagcaacctcagcagttcccatcaaggccacctccaccacaaccgaaagtatcatctcagggaaacttaattcctgcccgtcctgctcctgcacctcctttatatagttccctcacttgatttttttaaccttctttttgcaaatgtcttcagggaactgagctaatactttttttttttcttgatgttttcttgaaaagcctttctgttgcaactatgaatgaaaacaaaacaccacaaaacagacttcactaacacagaaaaacagaaactgagtgtgagagttgtgaaatacaaggaaatgcagtaaagccagggaatttacaataacatttccgtttccatcattgaataagtcttattcagtcatcggtgaggttaatgcactaatcatggattttttgaacatgttattgcagtgattctcaaattaactgtattggtgtaagatttttgtcattaagtgtttaagtgttattctgaattttctaccttagttatcattaatgtagttcctcattgaacatgtgataatctaatacctgtgaaaactgactaatcagctgccaataatatctaatatttttcatcatgcacgaattaataatcatcatactctagaatcttgtctgtcactcactacatgaataagcaaatattgtcttcaaaagaatgcacaagaaccacaattaagatgtcatattattttgaaagtacaaaatatactaaaagagtgtgtgtgtattcacgcagttactcgcttccatttttatgacctttcaactataggtaataactcttagagaaattaatttaatattagaatttctattatgaatcatgtgaaagcatgacattcgttcacaatagcactattttaaataaattataagctttaaggtacgaagtatttaatagatctaatcaaatatgttgattcatggctataataaagcaggagcaattataaaatcttcaatcaattgaacttttacaaaaccacttgagaatttcatgagcactttaaaatctgaactttcaaagcttgctattaaatcatttagaatgtttacatttactaaggtgtgctgggtcatgtaaaatattagacactaatattttcatagaaattaggctggagaaagaaggaagaaatggttttcttaaatacctacaaaaaagttactgtggtatctatgagttatcatcttagctgtgttaaaaatgaatttttactatggcagatatggtatggatcgtaaaattttaagcactaaaaattttttcataacctttcataataaagtttaataataggtttattaactgaatttcattagttttttaaaagtgtttttggtttgtgtatatatacatatacaaatacaacatttacaataaataaaatacttgaaattctcttttgtgtctcctagtagcttcctactcaactatttataatctcattaattaaaaagttataattttagataaaaattctagtcaaatttttacagatattatctcactaattttcagacttttgccaaagtgtgcacaatggctttttgttaataaagaacagattagttttgaagaaggcaaaaatttcagttttctgaagacagcatgttattttaacaatcaagtatacatattaaaaattgtgagcaatctcaaaaaaaaaaaaaaa

SEQ ID NO: 59 - DNA, 4768 bp, Homo sapiens
ggcggcttgcgcctgcgcggcgcggcgctgcggagaccgttggttcatttgcatgtccccgcctcgcgcggcggcggcggcgggtgaggagcctgaggcggcggcgggggtggctccgcgcgcggtggtctcgggggcaaaataacatggcagccagacgaattacacaggagacttttgatgctgtattacaagaaaaagccaaacgatatcacatggatgccagtggtgaggctgtaagcgaaactcttcagtttaaagctcaagatctcttaagggcagtcccaagatccagagcagagatgtatgatgacgtccacagcgatggcagatactccctcagtggatctgtagctcactctagagatgccggaagagaaggcctgagaagtgacgtatttccagggccttccttcagatcaagcaacccttccatcagtgatgacagctactttcgcaaagaatgtggccgggatctggaattttctcactctgattctcgggaccaggtcattggccaccggaaattggggcatttccgttctcaggactggaaatttgcgctccgtggttcttgggaacaagactttggccatccagtttctcaagagtcctcttggtcacaggagtatagttttggtccctctgcagttttgggggactttggatcttccaggctgattgagaaagagtgtttggagaaggagagtcgggattatgacgtggaccatcctggggaggctgactctgtgcttaggggcggcagtcaagtccaggccagaggtcgagctctaaacatcgttgaccaggaaggttccctcctaggaaagggggagactcagggcctgctcacagctaaggggggtgttgggaaacttgtcacattgagaaatgtgagcacaaaaaaaatacccaccgtgaatcgtattactcccaaaactcagggcactaaccaaatccagaaaaacactccaagtcctgatgtgaccctggggacaaacccagggacagaagatatccagttccccattcagaagatccctctggggctggatctgaagaatcttcggctccccagaagaaagatgagctttgacatcatagataagtctgatgttttttcaagatttgggatagaaataatcaaatgggcaggattccacaccataaaagatgatattaaattttcccaacttttccagactctctttgaacttgaaacagaaacctgtgctaaaatgcttgcctcattcaaatgttccttaaaaccagagcacagagatttttgcttttttactatcaaatttttaaagcactctgctttgaaaacacccagagttgataatgagtttttaaacatgcttttagacaaaggtgctgtgaagaccaaaaattgcttttttgaaatcataaagccttttgacaagtacataatgagacttcaagaccggcttctgaagagtgtcacacctttgcttatggcctgcaatgcctacgagctaagtgtcaagatgaagaccctcagtaaccccctggacttggctcttgccctagaaaccaccaactctctctgccggaagtctttggcccttttgggacagacattttccttggcctcttctttccggcaggagaaaatcttagaagctgtcggcctgcaagatatagctccctcacctgctgcgtttccaaacttcgaagactccactttgtttgggcgagagtacatagaccacctgaaggcctggctagtcagcagcggatgtcccctccaggttaagaaagccgaaccagagccgatgcgagaggaggagaaaatgattcctcctacgaaacctgaaattcaggccaaggctccaagtagtctgagtgatgctgtcccccagcgagcagatcacagggtagtgggcaccatcgaccagcttgtgaaacgtgtcatcgaaggcagcctgtctcccaaagagagaactcttctcaaagaggaccctgcttactggtttttgtctgatgaaaatagtctggagtataaatattacaagctgaagttggcagaaatgcagcggatgagcgagaacttgcgaggagccgaccagaagccgacctcagcagactgtgcagtgagggccatgctgtactcccgggctgtccgcaacctcaagaagaaactccttccgtggcagcggcgggggctcctccgtgctcaagggctccggggctggaaggcgaggagagcgaccaccgggacccagaccctcctatcctcaggcaccaggctgaaacaccacggccggcaggctccaggcctctcacaggcaaaaccatccctgccagacagaaatgatgctgccaaggactgcccgccagacccagttggaccttctcctcaggaccccagcttagaagcctcaggcccatcccccaagccagcaggagtggacatctctgaagcacctcagacctcttctccctgcccatctgctgacattgacatgaagacaatggagactgcagagaaactggctagatttgttgctcaggtgggaccagagatcgaacaattcagcatagaaaacagcaccgataaccctgacctgtggtttctacatgaccaaaatagttctgctttcaaattctatcgaaagaaagtgtttgaactatgtccatcaatttgtttcacgtcatctccgcacaaccttcacactggtggtggtgacaccacgggttctcaggagagccccgtggacctcatggaaggggaagcagagtttgaagacgagccccctccgcgggaggctgagctggagagcccagaggtgatgcctgaggaggaggacgaggacgatgaggatgggggagaggaggcccccgctcctggaggggcgggcaagtctgagggcagcacccctgccgacggccttcccggcgaggctgccgaggacgacctggctggagcacctgccttgtcacaggcctcctcaggtacctgcttccctcggaagaggatcagcagcaagtcattgaaggttggcatgattccagctcccaagagagtgtgtctcatccaggagccaaaagtccatgaaccagttcgaattgcctatgacaggcctcggggtcgtcccatgtccaaaaagaagaaacccaaggacttggacttcgcccagcagaagctgaccgataagaacctgggcttccagatgctgcagaagatgggctggaaggagggccatggcctgggctccctcggaaagggcatcagggagccggtcagcgtgggaaccccctcggaaggggaagggttgggtgctgacgggcaggagcacaaagaagacacattcgatgtgttccgacagaggatgatgcagatgtacagacacaagcgggccaacaaatagatcaaaaccactgatgtgaaagataagccttgaagcagcaattgcccttaaaacatcatccctgccctggatcggcctggagccagtgcccaagtacggtttggtgtgtacatgaaaacaaacgtctctgcagtctctggggcggaggtttcgctggcttttctttctctcaaagaaaaaaacatgcaccattttcaatgtgcttttgcctctcctctctgttcacatgcttttagcagcaagtcccctccaaatctgtcttggttccccttcagaaggtggcgctgcccccgaaaggcacctcagcctgtgagtgctgaggaaccagctcctctggctgattttccagttggactggccattgctctccagaagtgctctgttagcaaacgtgatgtggaaacgatcacagatggtgttttctcgttgttcgccagaatttatacgggggagacaaattcccggtaattaccaagtctgcactcgggtaccaaagctctgaagctctctgaacagttgccatacttgagttgatgaatgtgttattcatggtgtctcatctcatcaatgcatcttgagagacttaatgaaattttagcaacagtatagaatagctctatcgggtggggagtaatcattaaacagatgaaatcggccccagatttacatgtctctttagaatccacagtgtaagcaaactacagttacaaagggatgggggttgtaaaccctctgagactctgcacttttcgcacgtatggcatcgtcaagtgctgtcttattacagcctttgtaaggagaggcaggctcctcctggggtgggctctgcagctgctctatttccaggcatgtgatcgcccccgctctccagattccccagcactctgctgcgtgtaactccactcaattctccactcatccttccttgtgaagcaggatcgttgaagttttaagtatgggcaaaaatctggaaaacttaggatccctctgacaccccaggattaggggacacagcagtggctagggcatcagccacagaactgagcgggaaatgccacttgtattggctgtaaagaaatcctggctttgggccaggcacagtggctcaagcctgtaatcccagcactttaggaggttgaggcggatggatcacctgaggtcaggagtttgagaccagcctggccaacatggtgtaaccccgtctctactaaaaatacaaaaaaattagccaggcgtggtagcgggcacctgtaatcccagctactcaggaggctgaggcaggagaatcacttgaaccggggaggcagaggttgcagtgagctgagatcatgccactccactccagcctgggcgacagagcaagactccatctc

SEQ ID NO: 60 - DNA, 784 bp, Homo sapiens
gcccacgcgccagagtcgcagtgggcgggcctacgtgctccgcccgctgtgagcctgtccggcccccgcccgctccggagcaacccgcgagcttacaccggcttctctctgtcctcagcccgcgcgccgccatcgccgtcatgctgggcgccgctctccgccgctgcgctgtggccgcaaccacccgggccgaccctcgaggcctcctgcactccgcccggacccccggccccgccgtggctatccagtcagttcgctgctattcccatgggtcacaggagacagatgaggagtttgatgctcgctgggtaacatacttcaacaagccagatatagatgcctgggaattgcgtaaagggataaacacacttgttacctatgatatggttccagagcccaaaatcattgatgctgctttgcgggcatgcagacggttaaatgattttgctagtacagttcgtatcctagaggttgttaaggacaaagcaggacctcataaggaaatctacccctatgtcatccaggaacttagaccaactttaaatgaactgggaatctccactccggaggaactgggccttgacaaagtgtaaaccgcatggatgggcttccccaaggatttattgacattgctacttgagtgtgaacagttacctggaaatactgatgataacatattaccttatttgaacaagttttcctttattgagtaccaagccatgtaatggtaacttggactttaataaaagggaaatgagtttgaactgaaaaaaaaaaaaaaaaa

SEQ ID NO: 61 - DNA, 3670 bp, Homo sapiens
cgcagcaaacacatccgtagaaggcagcgcggccgccgagaaccgcagcgccgctcgcccgccgccccccaccccgccgccccgcccggcgaattgcgccccgcgcccctcccctcgcgcccccgagacaaagaggagagaaagtttgcgcggccgagcggggcaggtgaggagggtgagccgcgcgggaggggcccgcctcggccccggctcagcccccgcccgcgcccccagcccgccgccgcgagcagcgcccggaccccccagcggcggcccccgcccgcccagccccccggcccgccatgggcgccgcggcccgcaccctgcggctggcgctcggcctcctgctgctggcgacgctgcttcgcccggccgacgcctgcagctgctccccggtgcacccgcaacaggcgttttgcaatgcagatgtagtgatcagggccaaagcggtcagtgagaaggaagtggactctggaaacgacatttatggcaaccctatcaagaggatccagtatgagatcaagcagataaagatgttcaaagggcctgagaaggatatagagtttatctacacggccccctcctcggcagtgtgtggggtctcgctggacgttggaggaaagaaggaatatctcattgcaggaaaggccgagggggacggcaagatgcacatcaccctctgtgacttcatcgtgccctgggacaccctgagcaccacccagaagaagagcctgaaccacaggtaccagatgggctgcgagtgcaagatcacgcgctgccccatgatcccgtgctacatctcctccccggacgagtgcctctggatggactgggtcacagagaagaacatcaacgggcaccaggccaagttcttcgcctgcatcaagagaagtgacggctcctgtgcgtggtaccgcggcgcggcgccccccaagcaggagtttctcgacatcgaggacccataagcaggcctccaacgcccctgtggccaactgcaaaaaaagcctccaagggtttcgactggtccagctctgacatcccttcctggaaacagcatgaataaaacactcatcccatgggtccaaattaatatgattctgctccccccttctccttttagacatggttgtgggtctggagggagacgtgggtccaaggtcctcatcccatcctccctctgccaggcactatgtgtctggggcttcgatccttgggtgcaggcagggctgggacacgcggcttccctcccagtccctgccttggcaccgtcacagatgccaagcaggcagcacttagggatctcccagctgggttagggcagggcctggaaatgtgcattttgcagaaacttttgagggtcgttgcaagactgtgtagcaggcctaccaggtccctttcatcttgagagggacatggcccttgttttctgcagcttccacgcctctgcactccctgcccctggcaagtgctcccatcgccccggtgcccaccatgagctcccagcacctgactccccccacatccaagggcagcctggaaccagtggctagttcttgaaggagccccatcaatcctattaatcctcagaattccagtgggagcctccctctgagccttgtagaaatgggagcgagaaaccccagctgagctgcgttccagcctcagctgagtctttttggtctgcacccacccccccacccccccccccccgcccacatgctccccagcttgcaggaggaatcggtgaggtcctgtcctgaggctgctgtccggggccggtggctgccctcaaggtcccttccctagctgctgcggttgccattgcttcttgcctgttctggcatcaggcacctggattgagttgcacagctttgctttatccgggcttgtgtgcagggcccggctgggctccccatctgcacatcctgaggacagaaaaagctgggtcttgctgtgccctcccaggcttagtgttccctccctcaaagactgacagccatcgttctgcacggggctttctgcatgtgacgccagctaagcatagtaagaagtccagcctaggaagggaaggattttggaggtaggtggctttggtgacacactcacttctttctcagcctccaggacactatggcctgttttaagagacatcttatttttctaaaggtgaattctcagatgataggtgaacctgagttgcagatataccaacttctgcttgtatttcttaaatgacaaagattacctagctaagaaacttcctagggaactagggaacctatgtgttccctcagtgtggtttcctgaagccagtgatatgggggttaggataggaagaactttctcggtaatgataaggagaatctcttgtttcctcccacctgtgttgtaaagataaactgacgatatacaggcacattatgtaaacatacacacgcaatgaaaccgaagcttggcggcctgggcgtggtcttgcaaaatgcttccaaagccaccttagcctgttctattcagcggcaaccccaaagcacctgttaagactcctgacccccaagtggcatgcagcccccatgcccaccgggacctggtcagcacagatcttgatgacttccctttctagggcagactgggagggtatccaggaatcggcccctgccccacgggcgttttcatgctgtacagtgacctaaagttggtaagatgtcataatggaccagtccatgtgatttcagtatatacaactccaccagacccctccaacccatataacaccccacccctgttcgcttcctgtatggtgatatcatatgtaacatttactcctgtttctgctgattgtttttttaatgttttggtttgtttttgacatcagctgtaatcattcctgtgctgtgttttttattacccttggtaggtattagacttgcacttttttaaaaaaaggtttctgcatcgtggaagcatttgacccagagtggaacgcgtggcctatgcaggtggattccttcaggtctttcctttggttctttgagcatctttgctttcattcgtctcccgtctttggttctccagttcaaattattgcaaagtaaaggatctttgagtaggttcggtctgaaaggtgtggcctttatatttgatccacacacgttggtcttttaaccgtgctgagcagaaaacaaaacaggttaagaagagccgggtggcagctgacagaggaagccgctcaaataccttcacaataaatagtggcaatatatatatagtttaagaaggctctccatttggcatcgtttaatttatatgttatgttctaagcacagctctcttctcctattttcatcctgcaagcaactcaaaatatttaaaataaagtttacattgtagttattttcaaatctttgcttgataagtattaagaaatattggacttgctgccgtaatttaaagctctgttgattttgtttccgtttggatttttgggggaggggagcactgtgtttatgctggaatatgaagtctgagaccttccggtgctgggaacacacaagagttgttgaaagttgacaagcagactgcgcatgtctctgatgctttgtatcattcttgagcaatcgctcggtccgtggacaataaacagtattatcaaagagaaaaaaaaaaaaaaaaa

SEQ ID NO: 62 - DNA, 2604 bp, Homo sapiens
ggggaggagccaagggggcgagcaagctcggtggctgggtgggttggggcgttccgcgcgcccttcattgaagcggcggtggccgggctgggcgccggtagtggaaagcgacggcgcggctggaaaatgccagtccattcccgaggggataagaaggagaccaaccatcacgatgagatggaggtggactacgccgaaaatgaggggagcagctccgaggacgaggacactgagagctcgtcggtctccgaggatggagatagctcagaaatggatgatgaagactgtgaaagaagaagaatggaatgtttggatgaaatgtccaatcttgaaaaacagtttaccgatctcaaagatcaactttataaagaacgattaagtcaggtggatgcaaaactacaagaagtcatagctggaaaagcaccagaatacttggaaccgctggcaactttacaggaaaatatgcaaattcgtacaaaggtagcaggaatctatagagagctctgcttagaatctgtaaagaacaaatatgaatgtgaaattcaagcttctcgccagcattgtgagagcgaaaagctgttgctatatgatacagtccagagtgaactagaggagaagataagaaggcttgaagaggataggcacagcattgatattacctcagagctgtggaatgatgagcttcagtcaagaaaaaagaggaaggatcctttcagtcctgacaaaaagaagccagttgttgtttcaggtccatatatagtttatatgctacaagatcttgatattcttgaagactggacaacaattaggaaggcaatggctacattggggccacacagagtgaaaacggaaccacctgtgaaactggaaaaacatctgcacagtgctagatctgaagagggaagactatattatgatggtgaatggtatatacgtggacaaacaatatgtattgataaaaaagatgaatgtcctacaagtgctgtaattacaacaattaaccatgatgaagtttggtttaagaggcctgatggaagcaaatctaagctttacatttcacagctacagaaaggaaaatattcaattaaacattcataatcatgatttaagtgttatctaaatttaccttattagtgttaccaaatgtaagtgccatgagagtaaaaaaatgtattcaataacttaatattctcactgaatcatgagagaatgtgtatttgtaggtagtactctaaatagatctcattgatatgttattaaaagaaacagtaataaaaattttatcacgatccttacgttgatttgcctcttaggtccgatgaccaataggtattctgtatatggtaggggtttctttctaaacatttttctttggttttaaaaaaagttatgcaaatttgtcttatctttagtaaactatgactacatttatctgcaatttttaaaattttccatatctttgtcattcattgtgtgtttgtaaataaggccgatagaatgtttcctataaatggtttgtactagtacattagtgttaaaccagaactgaaatttaaacatatatatatatgaggatgtatatatggcatcatcagcttatttagaactgatggccataccttacaatcttgttttacccaaaattaagctattggggttgaaagctaaaaggagcacttttgtagaatagcaacttttcttttcctctttcttgattgtatggtggggtggtgacctatttttacaaattatacctaatgagtaaaattagtgtaaagtgataacatgcttctacctgtatttctagtgaccctttagcggcaggtatttatacctggtatttatgatgcagtatataagtggtgaacaataactgacagtattgtgcttgctgtacatgtctggtcttttgaaacagattttagtaagcattttccagaggtaaaactgtgtccttattctaattttattcctagggcaaagtagacagggattatttccttgaatctatttccaaattaatatttttttctttggtatttctacactttaaggccatttggtgcaatttagaaagtgttggcctcccttccgctagccacattcaaaattaacttccaaaacctcaggaacagtacaaagaattgaaaccctcaatatggcagcacagccggctgtagtgtatatttagggtacaccaaatcaggtattcctggtggtcttgtgcactttaatttctgttacaatgagttaagaggatgaggaagaaatctacttattaacacttactgcagaaatgtctgcattattccgtttgttttcttattattttacctctccaaacatcttcctgtgcagatcactacttcatagttgccaaattttaaaacacttaactgctgaaattcagtgtcagcaaagtgatattacgttgttctgtttctaattaaccttagcaaatgtacataatgtcaaaacccaatagtatttgacagtacttatgtatacaatgtttgataagcatttttaataagatttgtatttttaaatttagtatataataaaaagatgtgtttcagtgtgaaaaaaaaaaaaaaaaaaaaa

SEQ ID NO: 63 - DNA, 2732 bp, Homo sapiens
ctttcgcctcagtctcgagctctcgctggccttcgggtgtacgtgctccgggatcttcagcacccgcggccgccatcgccgtcgcttggcttcttctggactcatctgcgccacttgtccgcttcacactccgccgccatcatggtgaagctcgcgaaggcaggtaaaaatcaaggtgaccccaagaaaatggctcctcctccaaaggaggtagaagaagatagtgaagatgaggaaatgtcagaagatgaagaagatgatagcagtggagaagaggtcgtcatacctcagaagaaaggcaagaaggctgctgcaacctcagcaaagaaggtggtcgtttccccaacaaaaaaggttgcagttgccacaccagccaagaaagcagctgtcactccaggcaaaaaggcagcagcaacacctgccaagaagacagttacaccagccaaagcagttaccacacctggcaagaagggagccacaccaggcaaagcattggtagcaactcctggtaagaagggtgctgccatcccagccaagggggcaaagaatggcaagaatgccaagaaggaagacagtgatgaagaggaggatgatgacagtgaggaggatgaggaggatgacgaggacgaggatgaggatgaagatgaaattgaaccagcagcgatgaaagcagcagctgctgcccctgcctcagaggatgaggacgatgaggatgacgaagatgatgaggatgacgatgacgatgaggaagatgactctgaagaagaagctatggagactacaccagccaaaggaaagaaagctgcaaaagttgttcctgtgaaagccaagaacgtggctgaggatgaagatgaagaagaggatgatgaggacgaggatgacgacgacgacgaagatgatgaagatgatgatgatgaagatgatgaggaggaggaagaagaggaggaggaagagcctgtcaaagaagcacctggaaaacgaaagaaggaaatggccaaacagaaagcagctcctgaagccaagaaacagaaagtggaaggcacagaaccgactacggctttcaatctctttgttggaaacctaaactttaacaaatctgctcctgaattaaaaactggtatcagcgatgtttttgctaaaaatgatcttgctgttgtggatgtcagaattggtatgactaggaaatttggttatgtggattttgaatctgctgaagacctggagaaagcgttggaactcactggtttgaaagtctttggcaatgaaattaaactagagaaaccaaaaggaaaagacagtaagaaagagcgagatgcgagaacacttttggctaaaaatctcccttacaaagtcactcaggatgaattgaaagaagtgtttgaagatgctgcggagatcagattagtcagcaaggatgggaaaagtaaagggattgcttatattgaatttaagacagaagctgatgcagagaaaacctttgaagaaaagcagggaacagagatcgatgggcgatctatttccctgtactatactggagagaaaggtcaaaatcaagactatagaggtggaaagaatagcacttggagtggtgaatcaaaaactctggttttaagcaacctctcctacagtgcaacagaagaaactcttcaggaagtatttgagaaagcaacttttatcaaagtaccccagaaccaaaatggcaaatctaaagggtatgcatttatagagtttgcttcattcgaagacgctaaagaagctttaaattcctgtaataaaagggaaattgagggcagagcaatcaggctggagttgcaaggacccaggggatcacctaatgccagaagccagccatccaaaactctgtttgtcaaaggcctgtctgaggataccactgaagagacattaaaggagtcatttgacggctccgttcgggcaaggatagttactgaccgggaaactgggtcctccaaagggtttggttttgtagacttcaacagtgaggaggatgccaaagctgccaaggaggccatggaagacggtgaaattgatggaaataaagttaccttggactgggccaaacctaagggtgaaggtggcttcgggggtcgtggtggaggcagaggcggctttggaggacgaggtggtggtagaggaggccgaggaggatttggtggcagaggccggggaggctttggagggcgaggaggcttccgaggaggcagaggaggaggaggtgaccacaagccacaaggaaagaagacgaagtttgaatagcttctgtccctctgctttcccttttccatttgaaagaaaggactctggggtttttactgttacctgatcaatgacagagccttctgaggacattccaagacagtatacagtcctgtggtctccttggaaatccgtctagttaacatttcaagggcaataccgtgttggttttgactggatattcatataaactttttaaagagttgagtgatagagctaacccttatctgtaagttttgaatttatattgtttcatcccatgtacaaaaccattttttcctacaaatagtttgggttttgttgttgtttcttttttttgttttgtttttgttttttttttttttgcgttcgtggggttgtaaaagaaaagaaagcagaatgttttatcatggtttttgcttcagcggctttaggacaaattaaaagtcaactctggtgccagaaaaaaaaaaaaaaaa

SEQ ID NO: 64 - DNA, 1980 bp, Homo sapiens
ttgatcttcctgcctcagccttccaagtagctgggacttaaaggcgtgagccaccacacctgactaattttcgtattttttgtagagatggggtttcgccatgttgcccgggctgttctcgaactcctgagctcaagcaatctgcccacctcagcctcccaaagcgctgggattacaggcatgagccaccatcccagccaaaactataaaacttttagaaaagaacatagaagaaaatctttgggtcctgggggcaaagagctctgagacttgacatcaaaagcatgccgcataataggaaaatactagactttatttaggggttaagagtttagactctggactctctcagccttggtttcactagttagctctatcactaactacattgggcattgaaaattcctctgttgtcccacgtggtgcatggatgattgtagacgaggacactgagatcctgaaggcagaagtaatttctctaagcaacgttgttggttggtggcagagtctgggttacaacccctggtttcctgattccgagtccaagtgaaatacttttgcccctgcagtagaccctgctacagaggataaaaaggcacgtcataggctaggagaaaaattttgcctaccacatatgtaaccaaggactagcagctaggacatctgaagaattctcaacattcaacggggtagaagaatgaacgattcaatagaatatgggcaaaagacatgaagaggcattttaccaaacatagggtgctatggtccgaatgtttgcattctcctcaaattcctgtgttgaaatcctaacccccaaggtattggtattaggaggcaggggccctgggaagtgattaggtcataaaggtggagtcctcatggatgggattagtgtctttataaaagagacctttgccatgtgaggttacagtgagaagacatctgtctatgaagaaagtgggccctcaccaaacacagtctgctggcactttgcacttcaactccccagcttccagaactgtaaggaatataagtctgttgttggtaagccacccggtctatgatattttgttatagcagcccaaacagactaagacaggtgacaaataaacatgaaaagatgttcaacatcattagccattagggaaatgcagattaaaaccacagcgaaatatcatgatacagttttcagcatggctaaactagaaaatagtgacaccaccaaatgccgacaaggctgtggggaaactgggttgttcagacactgccactggggctgtagcgtactatagccactttcataaacagtttgtcagtttcttaaaaaactaaacctgcaactaccatatgacccagcaattacacccctgggcacctacccaagagaaatgaaaactcaacgtttgcgcaaaaacctgtgtaggaatgttcaagcagctttattcataatatgcccaaacaggaaacaactcagctgtccttcagtaggtaaatagttaagcaaattgtcatacccctgtgtcatggagcactacctagcaataacaaggagcaaattattgatacataacaatctggatgaatctccagagaattatgttgaatgaaaaaagccagcccctgaaggatacatactgtatgatgccatttacataacattcttgaaattctaaaattacagagatggggaacagatttgtggttaaagatggagccgggtgggaagaaagtaggtgtggctataaacgggtaacatgaaggatccttgtggtgatggaaatttctgtatttttattgtatccgtgtcagtatcctggttgtgatatggtaatacagttttgcaagatactacccttaggggaaatgaggtaagacctggcatctctctgtattatttcttaattgcatgtgaatctacaattatttcaaaataaaaagtatgattgaagtaactctcaggaagcttagcctactgtggat

SEQ ID NO: 65 - DNA, 1674 bp, Homo sapiens
gcggccgcccgccgccgcgctcctcctcctcctcctccagcgcccggcggcccgctgcctcctccgcccgacgccccgcgtcccccgccgcgccgccgccgccaccctctgcgccccgcgccgccccccggtcccgcccgccatgcccggcccggccgcgggcagcagggcccgggtctacgccgaggtgaacagtctgaggagccgcgagtactgggactacgaggctcacgtcccgagctggggtaatcaagatgattaccaactggttcgaaaacttggtcggggaaaatatagtgaagtatttgaggccattaatatcaccaacaatgagagagtggttgtaaaaatcctgaagccagtgaagaaaaagaagataaaacgagaggttaagattctggagaaccttcgtggtggaacaaatatcattaagctgattgacactgtaaaggaccccgtgtcaaagacaccagctttggtatttgaatatatcaataatacagattttaagcaactctaccagatcctgacagactttgatatccggttttatatgtatgaactacttaaagctctggattactgccacagcaagggaatcatgcacagggatgtgaaacctcacaatgtcatgatagatcaccaacagaaaaagctgcgactgatagattggggtctggcagaattctatcatcctgctcaggagtacaatgttcgtgtagcctcaaggtacttcaagggaccagagctcctcgtggactatcagatgtatgattatagcttggacatgtggagtttgggctgtatgttagcaagcatgatctttcgaagggaaccattcttccatggacaggacaactatgaccagcttgttcgcattgccaaggttctgggtacagaagaactgtatgggtatctgaagaagtatcacatagacctagatccacacttcaacgatatcctgggacaacattcacggaaacgctgggaaaactttatccatagtgagaacagacaccttgtcagccctgaggccctagatcttctggacaaacttctgcgatacgaccatcaacagagactgactgccaaagaggccatggagcacccatacttctaccctgtggtgaaggagcagtcccagccttgtgcagacaatgctgtgctttccagtggtctcacggcagcacgatgaagactggaaagcgacgggtctgttgcggttctcccacttttccataagcagaacaagaaccaaatcaaacgtcttaacgcgtatagagagatcacgttccgtgagcagacacaaaacggtggcaggtttggcgagcacgaactagaccaagcgaagggcagcccaccaccgtatatcaaacctcacttccgaatgtaaaaggctcacttgcctttggcttcctgttgacttcttcccgacccagaaagcatggggaatgtgaagggtatgcagaatgttgttggttactgttgctccccgagcccctcaactcgtcccgtggccgcctgtttttccagcaaaccacgctaactagctgaccacagactccacagtggggggacgggcgcagtatgtggcatggcggcagttacatattattattttaaaagtatatattattgaataaaaggttttaaaagaaaaaaaaaaaaaaaaaa

SEQ ID NO: 66 - DNA, 1980 bp, Homo sapiens
tcatttcaaaatttaggagttaatttatatttttaattgaatcagatttcataggcatagatattgtctgtcaatattcatatgtttatatagtggtaatttattaaacttcttaatccagatgtattattttagttatcttttttccactctagtgtcatagtttaaacttgttctttgatgttgagtatttattataacaatagttttttttgcctgcactctacaatgtatatttccagatataatttgtttatgtaacttgttgaccatttataatggggaaaaaagcttgctaaaagttctcaagatagctaggaaaatatcaatgagatatatctaaaagaaagggagaggggtttggaagattactgccactctctttccttatatatttcttaggacttctgaggtgcttttatgcttcttgttttgtgtaaagtatatatatatatatatatatatacacacacacaaagtatatataaacacaaagtatatatatacacacacatatacacaaagtatatatatatacacacaaagtatatatatatgtacacaaaatatatatatatacacaaaagtacttacaaggcatgttcttacctcaaaaagatgccaacttatttatgagaaatagatcctactttatggaaaagcaaaataggaacatgacaataaaccaatatgataaagcactgtcagagttcaaaaacacctatgatacctaaatgtactcatgtagtttggatcaaccagaaaggctggtgacaagaggtacagcttacttggtaacttaaagaataagaagggtttgaaagtgaagagacggtgagaatagctaaagaagaggaaaacagcatagcctacaagacaggagatgataaagtttaggggctatttagcaaataataaataaattgatttagaatagaagaaatcatgtgttggaaaagaggcttgaaacaagttcggtgttagagaagagaatattaagaaacaagtgggagataggacttctaaatgctgcactaaggatttcggatttattctcatggtaaaggagagccagccaaggcttttctacaggagagaggtataatcaagcagcgtgaagctgagtcagtagggggatcagtgagaataggaagacatcagggttggggaagatgaaagcttagtttaagcatgagttaattctaccaggatgatggtaattgttatattaagatagggatgaataagaaatatttcaaaggtataaaggataagcttgttgactgactgaacttaaggaacaaagtaaaaagcagagtcaaagtggcagaggctatagccagggacaacgactacatatccagccttttctatgtctcggggtgaagatgcctttcttattcactatttctctcttcaactcctccacaccaccatgcaaaatcatagcccatctatgcttgacgtgcctacatgtagaaacctgtgatgatctctccagcgagaaagcaggtttaatcccttgacagtccttgactcatagtaagttcttattttatttttaagaccggcatggatgacttttacttaatatctgttctttgccatttaatgctagagctgatgatattgagtggccatttcacaatatgtacctgttctgtgttaggaacacttctaaaaggggcttggaattattaatttatacaaaaacataaaatttcatcttgaatctataaacttgctttaatacaatgagtaaaagtgatcattttagctttggatctgaatttcacttgaaggcatgcacatgggattaggagttgggtgaataatcaggactggaaaagtaaacctagaaattattgacatggataaagagttgttgataccctgtgagaaggaactttgggaaatgtggatggaggaggacagaaaggagcagagaataaaagtatgaaagctagccctgtaggctca

SEQ ID NO: 67 - DNA, 1003 bp, Homo sapiens
gtctgcaggtatggatgttgttctcttttccctgtctttatttccttaccaatcggctgccatccgaggagctgaggaagcctagagctctcagaagcagtcctttgagctggtgtaggggcactcagaatggtccagcgtttgacataccgacgtaggctttcctacaatacagcctctaacaaaactaggctgtcccgaacccctggtaatagaattgtttacctttataccaagaaggttgggaaagcaccaaaatctgcatgtggtgtgtgcccaggcagacttcgaggggttcgtgctgtaagacctaaagttcttatgagattgtccaaaacaaagaaacatgtcagcagggcctatggtggttccatgtgtgctaaatgtgttcgtgacaggatcaagcgtgctttccttatcgaggagcagaaaatcgttgtgaaagtgttgaaggcacaagcacagagtcagaaagctaaataaaaaaatgaaacttttttgagtaataaaaatgaaaagacgctgtccaatagaaaaagttggtgtgctggagctacctcacctcagcttgagagagccagttgtgtgcatctctttccagttttgcatccagtgacgtctgcttggcatcttgagattgttatggtgagagtatttacacctcagcaaatgctgcaaaatcctgttttcccccagagagctggaggttaaatactaccagcacatccctagatactactcaagttacagtatatgatcactaatatagtatgctcttggtaccaggagctctgatatatatctggtacatgtttgataatgacttgattgttattataagtacttattaatacttcgattctgtaaagagtttagggtttgattttataaaatccaaaatgagccttttattgaatccagttctctatgtgaccagttctctgtatgaatggaagggaaaagaattaaaaatcttgcaaaggggaaaaaaaaaaaaaaaaaa

SEQ ID NO: 68 - DNA, 1980 bp, Homo sapiens
ggcaccgtgggagtttgcagctctggttgctccaagagcacaaatattaatgtagcacagatattaatattattaattagcacagacattaatgtagtcacagaaagaaaaagagatgaaaaagagacaggttcttcactgcatgagaggctccgtttgggatctctcagaaatgtggaagcagaggctacagcacaagcctgggttattgctagtagcaagacagaaaataaggcttgggtaagctgtagttatagttacaatggaaatgactggcccaagagagtgctacagattacatagcagctactaagaaaaaggacaggcagaaggggtaggcaagacatgttctctggctgttgcagccaccaaaaagccaggatacaaaggcagggagttatctgaactgccttcctggagggtcatgcatttaggatccgactcattgactcttttccttaattttgctctgtacatttctctaagagggctaaccagtgtcaaggtttgataatatctgaaatggtattctggtgccaaagtatcatctcacaaattatttagaaattgcaaagagaaaatatattttataatccagatatctggcagttaaccacatgaccaaatttagcatcactaacagtaggacaactagatattatatacctcttgctgtgatatactatgaagtacacatcatcaactatgaagtattattttttttttctttgagatagggtcatgctctgtcgcccaatttagagtgcagcgatgcaatcatagctcactgcagctttgacctcccagtctcaagtgatcctcccacctcagcctccctagtagctgggactacagatgtgttccaccacacctggctaatttttatatattttttgtagtgatggggtttcaccatgttgcacaggctggtcttgaactcctgggcttaagcaatctgcctgaaagttctgggattataggcatgagccactgtgtccagactatgaagtattcttgccaaaactgatcaacctaaatctaatcaagcttctgggccagaactgtccaatagcaatgtaatgtcagctacatgtaatttaaaattttctagttgccaccaaaagcacagaaaagaaaaaatagataaattgtgctacatcaagattaaatacttctttgcatcaaaggacataatcaacacagagaaaaggcaaaccactgaatgggagaaaatatttgcaaattgatattcataatatgtaaagaatctttacaactcaacacccacaaaataaaaaaaaagattaaaaaatggggaaaggacttgaatagacatttctccaaagaagatgtacaacttgccaataagcacaagaaaagactaattatgagggaaatgcaaattaaaaccacaatgagatcaaacacattatgttggctatcataaaaagaaagtgccaggcgcaatgatcacagctactcaacaggctgggtggaagaatcccttgagaccaggagttagaggctgcagtgtgttatgatcatgcctgtgaatagccactgcactccaacataggtaacatagcaagccccatccataaaataaaataaaataaaataaaataaaggcaacaaaaaataacaagtattggtaaggatgtggagaaattggaaccctcgtgcattgctggtgggtgtgtaaaaaggtatggctgctgtgaaaaatgggatggctattcttcaaaaaattaaccacagaattactatatgatccagcaatcccacttctgcatacacatccaaaagaagtggactcaaggactcagacagatatttgtacccccctgttcatagcagcattatttacaatagccaaaaagtagaagcaaccacagattcatcaatgtatgaatggataaacaaaatgtggcatatacacatagtgggatatcattcagctttaaaaagggaggaaattct

SEQ ID NO: 69 - DNA, 1032 bp, Homo sapiens
cccgcaccccctgggattgtgggaaatgtagttttttgcctccgtaagggaccaggcggagctgaggaaccgcgcgaggactgggaccgtgattccactaaccggaaaccgtcgcctttcgggcccggcggggcctgagccaatgcagaatcgggggccgcgaggacgccagcgggcgctgtgcgtaggaaccgccgggtggccgctgccgatcggggccgacttggggacggaccggaagtgcccgagggcggccgcagaacggtcaatttgagccgcgtcgagctcccctgggacctgtggccgccgcccacagaccatgctcctggggcgcctgacttcccagctgttgagggccgttccttgggcaggcggccgcccgccttggcccgtctctggagtgctgggcagccgggtctgcgggcccctttacagcacatcgccggccggcccaggtagggcggcctctctccctcgcaagggggcccagctggagctggaggagatgctggtccccaggaagatgtccgtcagccccctggagagctggctcacggcccgctgcttcctgcccagactggataccgggaccgcagggactgtggctccaccgcaatcctaccagtgtccgcccagccagataggggaaggggccgagcagggggatgaaggcgtcgcggatgcgcctcaaattcagtgcaaaaacgtgctgaagatccgccggcggaagatgaaccaccacaagtaccggaagctggtgaagaagacgcggttcctgcggaggaaggtccaggagggacgcctgagacgcaagcagatcaagttcgagaaagacctgaggcgcatctggctgaaggcggggctaaaggaagcccccgaaggctggcagacccccaagatctacctgcggggcaaatgagtctggcgccgcccttcccgcccgttgctgctgtgatccgtagtaataaattctcagaggactcagcctttaaaaaaaaaaaaaaaaaaaaaaaaaaaaaaaaaaaaaaaaaa

SEQ ID NO: 70 - DNA, 3251 bp, Homo sapiens
cagcaactatgaaataatcgtagtatgagaggcagagatcggggcgagacaatggggatgtgggcgcgggagccccgttccggcttagcagcacctcccagccccgcagaataaaaccgatcgcgccccctccgcgcgcgccctcccccgagtgcggagcgggaggaggcggcggcggccgaggaggaggaggaggaggccccggaggaggaggcgttggaggtcgaggcggaggcggaggaggaggaggccgaggcgccggaggaggccgaggcgccggagcaggaggaggccggccggaggcggcatgagacgagcgtggcggccgcggctgctcggggccgcgctggttgcccattgacagcggcgtctgcagctcgcttcaagatggccgcttggctcgcattcattttctgctgaacgacttttaactttcattgtcttttccgcccgcttcgatcgcctcgcgccggctgctctttccgggattttttatcaagcagaaatgcatcgaacaacgagaatcaagatcactgagctaaatccccacctgatgtgtgtgctttgtggagggtacttcattgatgccacaaccataatagaatgtctacattccttctgtaaaacgtgtattgttcgttacctggagaccagcaagtattgtcctatttgtgatgtccaagttcacaagaccagaccactactgaatataaggtcagataaaactctccaagatattgtatacaaattagttccagggcttttcaaaaatgaaatgaagagaagaagggatttttatgcagctcatccttctgctgatgctgccaatggctctaatgaagatagaggagaggttgcagatgaagataagagaattataactgatgatgagataataagcttatccattgaattctttgaccagaacagattggatcggaaagtaaacaaagacaaagagaaatctaaggaggaggtgaatgataaaagatacttacgatgcccagcagcaatgactgtgatgcacttaagaaagtttctcagaagtaaaatggacatacctaatactttccagattgatgtcatgtatgaggaggaacctttaaaggattattatacactaatggatattgcctacatttatacctggagaaggaatggtccacttccattgaaatacagagttcgacctacttgtaaaagaatgaagatcagtcaccagagagatggactgacaaatgctggagaactggaaagtgactctgggagtgacaaggccaacagcccagcaggaggtattccctccacctcttcttgtttgcctagccccagtactccagtgcagtctcctcatccacagtttcctcacatttccagtactatgaatggaaccagcaacagccccagcggtaaccaccaatcttcttttgccaatagacctcgaaaatcatcagtaaatgggtcatcagcaacttcttctggttgatacctgagactgttaaggaaaaaaattttaaacccctgatttatatagatatcttcatgccattacagctttctagatgctaatacatgtgactatcgtccaatttgctttcttttgtagtgacattaaatttggctataaaagatggactacatgtgatactcctatggacgttaattgaaaagaaagattgttgttataaagaattggtttcttggaaagcaggcaagactttttctctgtgttaggaaagatgggaaatggtttctgtaaccattgtttggatttggaagtactctgcagtggacataagcattgggccatagtttgttaatctcaactaacgcctacattacattctccttgatcgttcttgttattacgctgttttgtgaacctgtagaaaacaagtgctttttatcttgaaattcaaccaacggaaagaatatgcatagaataatgcattctatgtagccatgtcactgtgaataacgatttcttgcatatttagccattttgattcctgtttgatttatacttctctgttgctacgcaaaaccgatcaaagaaaagtgaacttcagttttacaatctgtatgcctaaaagcgggtactaccgtttattttactgacttgtttaaatgattcgcttttgtaagaatcagatggcattatgcttgttgtacaatgccatattggtatatgacataacaggaaacagtattgtatgatatatttataaatgctataaagaaatattgtgtttcatgcattcagaaatgattgttaaaattctcccaactggttcgacctttgcagatacccataacctatgttgagccttgcttaccagcaaagaatatttttaatgtggatatctaattctaaagtctgttccattagaagcaattggcacatctttctatactttatatacttttctccagtaatacatgtttactttaaaaattgttgcagtgaagaaaaacctttaactgagaaatatggaaaccgtcttaattttccattggctatgatggaattaatattgtattttaaaaatgcatattgatcactataattctaaaacaattttttaaataaaccagcaggttgctaaaagaaggcattttatctaaagttattttaataggtggtatagcagtaattttaaatttaagagttgcttttacagttaacaatggaatatgccttctctgctatgtctgaaaatagaagctatttattatgagcttctacaggtatttttaaatagagcaagcatgttgaatttaaaatatgaataaccccacccaacaattttcagtttattttttgctttggtcgaacttggtgtgtgttcatcacccatcagttatttgtgagggtgtttattctatatgaatattgtttcatgtttgtatgggaaaattgtagctaaacatttcattgtccccagtctgcaaaagaagcacaattctattgctttgtcttgcttatagtcattaaatcattacttttacatatattgctgttacttctgctttctttaaaaatatagtaaaggatgttttatgaagtcacaagatacatatatttttattttgacctaaatttgtacagtcccattgtaagtgttgtttctaattatagatgtaaaatgaaatttcatttgtaattggaaaaaatccaataaaaaggatattcatttagaaaaaaaaaaaaaaaaaaaaaaaaaaaaaaaaaaaaaaaaaaaaaaaaaaaa

SEQ ID NO: 71 - DNA, 1155 bp, Homo sapiens
cgcctgtcttttccgtgctacctgcagaggggtccatacggcgttgttctggattcccgtcgtaacttaaagggaaattttcacaatgtccggagcccttgatgtcctgcaaatgaaggaggaggatgtccttaagttccttgcagcaggaacccacttaggtggcaccaatcttgacttccagatggaacagtacatctataaaaggaaaagtgatggcatctatatcataaatctcaagaggacctgggagaagcttctgctggcagctcgtgcaattgttgccattgaaaaccctgctgatgtcagtgttatatcctccaggaatactggccagagggctgtgctgaagtttgctgctgccactggagccactccaattgctggccgcttcactcctggaaccttcactaaccagatccaggcagccttccgggagccacggcttcttgtggttactgaccccagggctgaccaccagcctctcacggaggcatcttatgttaacctacctaccattgcgctgtgtaacacagattctcctctgcgctatgtggacattgccatcccatgcaacaacaagggagctcactcagtgggtttgatgtggtggatgctggctcgggaagttctgcgcatgcgtggcaccatttcccgtgaacacccatgggaggtcatgcctgatctgtacttctacagagatcctgaagagattgaaaaagaagagcaggctgctgctgagaaggcagtgaccaaggaggaatttcagggtgaatggactgctcccgctcctgagttcactgctactcagcctgaggttgcagactggtctgaaggtgtacaggtgccctctgtgcctattcagcaattccctactgaagactggagcgctcagcctgccacggaagactggtctgcagctcccactgctcaggccactgaatgggtaggagcaaccactgactggtcttaagctgttcttgcataggctcttaagcagcatggaaaaatggttgatggaaaataaacatcagtttctaaaagttgtcttcatttagtttgctttttactccagatcagaatacctgggattgcatatcaaagcataataataaatacatgtctcgacatgagttgtacttctaaaaaaaaaaa

SEQ ID NO: 72 - DNA, 558 bp, Homo sapiens
ggagtttcacttttgttgcccaggattgagtgcagtgccccgatcttggctcactacaacctctgcctcctgggttcaagcgactctcctgcctcagtgtcctgagtagctgggattacaggcgtctgccaccacgcccggctaattttgtatttttagtagagaacaggtttcactatgttggtcaggctggtcttgaactcctgacctcagcgcatccagaattttagacggggcccccagggtgaggtcttggcaccctccagtagagaagaagggacatgggccatacgtggggtgtcctttctgggagccttgcgtcccttacctgcctagccagggattgcacctcacagcacgcagccagcaggaacggcaccgtgatctgatttcacctgcgggccctgggccctgggggtgtttgacaattggggcatatcacagtgtgagctagtcccgtctcgggggtttggaggctccacgtggccgtggtacaggagcaggcagttccatcctctggcctggatcaggctctgcacacggaggcctgtgggccag

SEQ ID NO: 73 - DNA, 7130 bp, Homo sapiens
gaattccacattgtttgctgcacgttggattttgaaatgctagggaactttgggagactcatatttctgggctagaggatctgtggaccacaagatctttttatgatgacagtagcaatgtatctgtggagctggattctgggttgggagtgcaaggaaaagaatgtactaaatgccaagacatctatttcaggagcatgaggaataaaagttctagtttctggtctcagagtggtgcagggatcagggagtctcacaatctcctgagtgctggtgtcttagggcacactgggtcttggagtgcaaaggatctaggcacgtgaggctttgtatgaagaatcggggatcgtacccaccccctgtttctgtttcatcctgggcatgtctcctctgcctttgtcccctagatgaagtctccatgagctacaagggcctggtgcatccagggtgatctagtaattgcagaacagcaagtgctagctctccctccccttccacagctctgggtgtgggagggggttgtccagcctccagcagcatggggagggccttggtcagcctctgggtgccagcagggcaggggcggagtcctggggaatgaaggttttatagggctcctgggggaggctccccagccccaagcttaccacctgcacccggagagctgtgtcaccatgtgggtcccggttgtcttcctcaccctgtccgtgacgtggattggtgagaggggccatggttggggggatgcaggagagggagccagccctgactgtcaagctgaggctctttcccccccaacccagcaccccagcccagacagggagctgggctcttttctgtctctcccagccccacttcaagcccatacccccagcccctccatattgcaacagtcctcactcccacaccaggtccccgctccctcccacttaccccagaactttctccccattgcccagccagctccctgctcccagctgctttactaaaggggaagttcctgggcatctccgtgtttctctttgtggggctcaaaacctccaaggacctctctcaatgccattggttccttggaccgtatcactggtccatctcctgagcccctcaatcctatcacagtctactgacttttcccattcagctgtgagtgtccaaccctatcccagagaccttgatgcttggcctcccaatcttgccctaggatacccagatgccaaccagacacctccttcttcctagccaggctatctggcctgagacaacaaatgggtccctcagtctggcaatgggactctgagaactcctcattccctgactcttagccccagactcttcattcagtggcccacattttccttaggaaaaacatgagcatccccagccacaactgccagctctctgattccccaaatctgcatccttttcaaaacctaaaaacaaaaagaaaaacaaataaaacaaaaccaactcagaccagaactgttttctcaacctgggacttcctaaactttccaaaaccttcctcttccagcaactgaacctggccataaggcacttatccctggttcctagcaccccttatcccctcagaatccacaacttgtaccaagtttcccttctcccagtccaagaccccaaatcaccacaaaggacccaatccccagactcaagatatggtctgggcgctgtcttgtgtctcctaccctgatccctgggttcaactctgctcccagagcatgaagcctctccaccagcaccagccaccaacctgcaaacctagggaagattgacagaattcccagcctttcccagctccccctgcccatgtcccaggactcccagccttggttctctgcccccgtgtcttttcaaacccacatcctaaatccatctcctatccgagtcccccagttccccctgtcaaccctgattcccctgatctagcaccccctctgcaggcgctgcgcccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaagtgagtaggggcctggggtctggggagcaggtgtctgtgtcccagaggaataacagctgggcattttccccaggataacctctaaggccagccttgggactgggggagagagggaaagttctggttcaggtcacatggggaggcagggttggggctggaccaccctccccatggctgcctgggtctccatctgtgtccctctatgtctctttgtgtcgctttcattatgtctcttggtaactggcttcggttgtgtctctccgtgtgactattttgttctctctctccctctcttctctgtcttcagtctccatatctccccctctctctgtccttctctggtccctctctagccagtgtgtctcaccctgtatctctctgccaggctctgtctctcggtctctgtctcacctgtgccttctccctactgaacacacgcacgggatgggcctggggggaccctgagaaaaggaagggctttggctgggcgcggtggctcacacctgtaatcccagcactttgggaggccaaggcaggtagatcacctgaggtcaggagttcgagaccagcctggccaactggtgaaaccccatctctactaaaaatacaaaaaattagccaggcgtggtggcgcatgcctgtagtcccagctactcaggaggctgagggaggagaattgcttgaacctgggaggttgaggttgcagtgagccgagaccgtgccactgcactccagcctgggtgacagagtgagactccgcctcaaaaaaaaaaaaaaaaaaaaaaaaaaaaaagaaaagaaaagaaaagaaaaggaatcttttatccctgatgtgtgtgggtatgagggtatgagagggcccctctcactccattccttctccaggacatccctccactcttgggagacacagagaagggctggttccagctggagctgggaggggcaattgagggaggaggaaggagaagggggaaggaaaacagggtatgggggaaaggaccctggggagcgaagtggaggatacaaccttgggcctgcaggccaggctacctacccacttggaaacccacgccaaagccgcatctacagctgagccactctgaggcctcccctccccggcggtccccactcagctccaaagtctctctcccttttctctcccacactttatcatcccccggattcctctctacttggttctcattcttcctttgacttcctgcttccctttctcattcatctgtttctcactttctgcctggttttgttcttctctctctctttctctggcccatgtctgtttctctatgtttctgtcttttctttctcatcctgtgtattttcggctcaccttgtttgtcactgttctcccctctgccctttcattctctctgtccttttaccctcttcctttttcccttggtttctctcagtttctgtatctgcccttcaccctctcacactgctgtttcccaactcgttgtctgtatttttggcctgaactgtgtcttccccaaccctgtgtttttctcactgtttctttttctcttttggagcctcctccttgctcctctgtcccttctctctttccttatcatcctcgctcctcattcctgcgtctgcttcctccccagcaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagtgtacgcctgggccagatggtgcagccgggagcccagatgcctgggtctgagggaggaggggacaggactcctgggtctgagggaggagggccaaggaaccaggtggggtccagcccacaacagtgtttttgcctggcccgtagtcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcggtgagtcatccctactcccaagatcttgaggggaaaggtgagtggggaccttaattctgggctggggtctagaagccaacaaggcgtctgcctcccctgctccccagctgtagccatgccacctccccgtgtctcatctcattccctccttccctcttctttgactccctcaaggcaataggttattcttacagcacaactcatctgttcctgcgttcagcacacggttactaggcacctgctatgcacccagcactgccctagagcctgggacatagcagtgaacagacagagagcagcccctcccttctgtagcccccaagccagtgaggggcacaggcaggaacagggaccacaacacagaaaagctggagggtgtcaggaggtgatcaggctctcggggagggagaaggggtggggagtgtgactgggaggagacatcctgcagaaggtgggagtgagcaaacacctgccgcaggggaggggagggccctgcggcacctgggggagcagagggaacagcatctggccaggcctgggaggaggggcctagagggcgtcaggagcagagaggaggttgcctggctggagtgaaggatcggggcagggtgcgagagggaagaaaggacccctcctgcagggcctcacctgggccacaggaggacactgcttttcctctgaggagtcaggaactgtggatggtgctggacagaagcaggacagggcctggctcaggtgtccagaggctgccgctggcctccctatgggatcagactgcagggagggagggcagcagggatgtggagggagtgatgatggggctgacctgggggtggctccaggcattgtccccacctgggcccttacccagcctccctcacaggctcctggccctcagtctctcccctccactccattctccacctacccacagtgggtcattctgatcaccgaactgaccatgccagccctgccgatggtcctccatggctccctagtgccctggagaggaggtgtctagtcagagagtagtcctggaaggtggcctctgtgaggagccacggggacagcatcctgcagatggtcctggcccttgtcccaccgacctgtctacaaggactgtcctcgtggaccctcccctctgcacaggagctggaccctgaagtcccttccctaccggccaggactggagcccctacccctctgttggaatccctgcccaccttcttctggaagtcggctctggagacatttctctcttcttccaaagctgggaactgctatctgttatctgcctgtccaggtctgaaagataggattgcccaggcagaaactgggactgacctatctcactctctccctgcttttacccttagggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaactccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtgggatacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctcttatactgtggtttattctggtttgttacattgacaggagacacactgaaatcagcaaaggaaacaggcatctaagtggggatgtgaagaaaacagggaaaatctttcagttgttttctcccagtggggtgttgtggacagcacttaaatcacacagaagtgatgtgtgaccttgtgtatgaagtatttccaactaaggaagctcacctgagccttagtgtccagagttcttattgggggtctgtaggataggcatggggtactggaatagctgaccttaacttctcagacctgaggttcccaagagttcaagcagatacagcatggcctagagcctcagatgtacaaaaacaggcattcatcatgaatcgcactgttagcatgaatcatctggcacggcccaaggccccaggtataccaaggcacttgggccgaatgttccaagggattaaatgtcatctcccaggagttattcaagggtgagccctgtacttggaacgttcaggctttgagcagtgcagggctgctgagtcaaccttttactgtacaggggggtgagggaaagggagaagatgaggaaaccgcctagggatctggttctgtcttgtggccgagtggaccatggggctatcccaagaaggaggaattc

SEQ ID NO: 74 - protein, 16 aa, Homo sapiens
SEGRTVTNKVSRKYTG

SEQ ID NO: 75 - DNA, 439 bp, Homo sapiens
tgtgtgggtatgagggtatgagagggcccctctcactccattccttctccaggacatccctccactcttgggagacacagagaagggctggttccagctggagctgggaggggcaattgagggaggaggaaggagaagggggaaggaaaacagggtatgggggaaaggaccctggggagcgaagtggaggatacaaccttgggcctgcaggccaggctacctacccacttggaaacccacgccaaagccgcatctacagctgagccactctgaggcctcccctccccggcggtccccactcagctccaaagtctctctcccttttctctcccacactctatcatcccccggattcctctctacttggttctcattcttcctttgacttcctgatcctgtgtattttcggctcaccttgatttgtcactgttctcccctc

SEQ ID NO: 76 - DNA, 5830 bp, Homo sapiens
tttagaagtacaactcatgtcgagacatgtatttattattatgctttgatatgcaatcccaggtattctgatctggagtaaaaagcaaactaaatgaagacaacttttagaaactgatgtttattttccatcaaccatttttccatgctgcttaagagcctatgcaagaacagcttaagaccagtcagtggttgctcctacccattcagtggcctgagcagtgggagctgcagaccagtcttccgtggcaggctgagcgctccagtcttctgttaagagaataccaaaaaaagccatcagctctgtcctcaatgtagtatttttccttaccccactcccaacagcagtctatcagatgctgtagcacactggaatgaaaacctcttaaagctagtataaaagtcacagagatcattaaagttagaataaaactgagaagttgcaaaatatcagcttgattcacctctatcctgatacataccagtagggaattgctgaataggcacagagggcacctgtacaccttcagaccagtctgcaacctcaggctgagtagcagtgaactcaggagcgggagcagtccattcaccctgaaattcctccttggtcactgccttctcagcagcagcctgctcttctttttcaatctgggttacaaaaaaaaaagaggaaagacttacagggactgacaagttttgcttcctagaagcacagtccaagtcgtccaatttgcttatgtaaccacaagcctttggagaagcttacctcttcaggatctctgtagaagtacagatcaggcatgacctcccatgggtgttcacgggaaatggtgccacgcatgcgcagaacttcccgagccagcatccaccacatcaaacccactgagtgagctccctgccaagagtggcagattattaaaaagtgacacttggtcaaacccaaacagcaagcaccttcctggctcttacatcatctaggtaccaaaagtacagacatctcttagttgaaaagcaattgtaacagggagcattataaatagggattttcaaccttgtctgcacactggaatcatagggattgaaaaaaaaatgcctggacactggcatttaaaaaaaatatctgggcaattcttatgcacaactagggttacaaatcaaccaatctgaaccacacacaacccacctttttagcatcttatagtagaacccatcaagttggtgtgaaaacaaaggccaagtgcctgaggcctgagcaagaaacatggacaaaggcagaagaacacactattttccaaatgttttcatttctccacaaagctgtcatgcagtaatgttatacttgtagcaagaattactatcaaactccactcattgggccagagacccttgtggtgttagcgaaaacttgcccctcatgtagcacactacctacacttgcgagttcatcggccagtcagtagcctcaactccaagctctaacaatgtgcacaggaatgttttttctagagcacgcattcacaaactctagatcctaaaatcattaccttgttgttgcatgggatggcaatgtccacatagcgcagaggagaatctgtgttacacagcgcaatggtaggtaggttaacataagatgcctccgtgagaggctggtggtcagccctggggtcagtaaccacaagaagccgtggctcccggaaggctgcctggatctggttagtgaaggttccaggagtgaagcggccagcaattggagtggctccagtggcagcagcaaacttcagcacagccctctttgaagaatttaaaagttagtggtactcgatattccacagcaacaatcctggcacccagtaagcacttctgggcactggccaacttaatcactactactccatgtaaagcaggcacatacccagctttccttaacctctcttacttctccagagatttcctgcatacattaaaaaagacaggcttgtttttctactatatatcccgagcacctagcaacactacctggcccaatctagatgctcagtatatttttctcaagcaagcaagaactgtcactcagttcattctacagcaataactaactgccttttttctactgtgtaccaaactatgtggtcctagtaacaagtcagtttcaggaataaccctctgcaaaaacacacatgggaacccgtcctgtcaacattgaaataaagtcttactacataatttttaaatctgtcaaaatagttaagatgaataaaatccctgttagaactcttagaattcaagaaatctactagttttctcaacagaaaaattattaaaatagagctgtggcaaaaaagattcaagttttactaactagtcatttcttaaaaatacaaattaaggtatttttcattcaagttggcagaggttctctttgagacaaggcctcactctgtcactcaggctgggatgcagtggacagtcttggctccctgctgcctcaacctcctgtgttctagcaatcttcccaccttagcctcagctgagactacaggcacgccaacaccacacccagctcgtttttgttaattttggtagagacggagttttgccacgttgcccaccctggtctcgaacttctgggctcaagcaagccaccagcctcagcctcacaaagtaccccatattacaaaagcatgagccacccatgactggcttttttaagaaacaggatctcactctgtgccccaggcttggagtgcagtggcaaaatcataacttactgcagcccccagctcctggacttgagggatctcctggctcagactcccaattaacgggaactatgggtgcatgccgtcatgactggctaagattcttaaaaaatttctgtcccagtatttcattgttttaaggtaaaaactttgactatgggaaaatgtgataagccactttaatagaatttctagatatttagtggcacaagtctacacaaaatgaaattataaagttcaacatactgccagatgcccagcatgtatgttgaaaaggtcttacaggctgggcgcagtggcttacgcctgtaatcccaacactttgggaggccgaggtgggaggatcacttgagatcaggcgtttaagaccagcctggccaaatggtaaaaccatgtctctactaaaaatacaaaaatcagtcaggcatggtggcacatgcctgtaattccagctactcgggaggatgaggcaggagaatcccttgagcttaggaggcaaaggctgcaatgagccgatatcacaccactgcagtccaggatggacaacaggcaacagagcgagagactgtcttgaaggaaaaaaaaaaaaaaaaaaaaaaaggaaggtcataggtcataagggcgatttctatggctcagtccaacttaagatctcttaacagtggagtttgagttttctattttctttttttttttttttgagacagagtctcggctctgtcacccaggctggagtacagtggcgcatctcagctcactgcaagctccgcccaccgggttcacgccattctccactttggcctcctgagtagctgggactacaggcacccgcccccacgcccaactaattttttgtatttttagtagagatggggcttcaccgtggtgtggatctcctgacctcgtaatacgccggactccacctcccaaagtgctgggattacaggcgtagccaccgcgcccggcctgagttatttctgttttcttatgtctttctatgtctcacaatgtctgtaagctttatttctataatataaaaactaaccactgttccacaaacctggccagtattcctggaggatataacactgacatcagcagggttttcaatggcaacaattgcacgagctgccagcagaagcttctcccaggtcctcttgagatttatgatatagatgcctaagtgagggcagaaggatttgtaacacttgacctggctgtgcaaacattcttctcaagtttactctatgtggcagcgagtactaccaaactccagtcatagaggcaagctctactggtgttagcgaaaatcctgccattgatatagcacacttccgacactcagaagttcatgagccaagactggcctccaccctgagctttaatagtgtgcagcctagattcagttggctaaatccatctttcctttctctttccctgtacttattccatcattggttcttaataatccacttcctggctttctgccactctttcccttttataatctgcactgatgcaagattcatcctaaaactcctttacagcatgtcactcccaagtccttggtagctttatctctaaaataagtaagtgccttagccaacttaatgccttccatcctctggttcttccaaaccatattgttcattcccacctcttaccccactttccactcaaaattcagttcaatagcctgtgtaagtacctatgaagttgaaggcactatgcgagctattagaaactcagccaatgaactttctcatgttcatgacccaacccaagtcccgtaactcagacaactgacatattcctcccctggagcagaaatcttttctctcaataacaagaacatttatttaaagtatgttttcaacgtaggggtctggcttcatttaaggaagatagaaagaaattacccagaactaaccacgagaatagcaagctcaaaatttgtacttacagctggagtaacaaaaaattaaagcaatgactaaccatcacttttccttttatagatgtactgttccatctggaagtcaagattggtgccacctaagtgggttcctgctgcaaggaacttaaggacatcctcctccttcatttgcaggacatcaagggctccggacattgtgaaaatttccctttaagttacgacgggaatctgaaataagagagcaacctatttcttttcattccattgagttaagtagggcacacccttaaaagcaaccaaaaactccatctgctatagtgtcaggcattgagcttacagtgtagtgaagggtaaacagaaagttaacccatacatgtcgtgtattacggaacagcgcacatcttactgaggaaatagtgtggacggcagagtaaccgagtaataaaactacacgaaattagcgaatggaaattcaacacttctcaagataggaattaaggaaaagttgtggatgcgccaagttaacaactagatacaaaagagcagccatgtttggaatatgcaaactaaaggcaaaaaaggcatgaattgttttcagacattttcaagcagattgtaggcggatgctttggaattacttcgctgtgctgtcaccacttgtttgtctaaaaacccgagaaccttaactcctgacccgcacggcgcgaagggaaaacccgaccagcccaagtagctggtgtaaatcagcgcctgggcctagactcaccgccgtgtgggactccgaagggatagcaacacaggatgttagcccgctttctgcactcccgcccacccgcggcctggtccgaaccccagctcggagcctccgctggagggcggcctccacccgctcccggggcactctgggaacccgccagcccgagcctggccacgtgcgggccgcaggcctaaggtggggcgcgccgggcgggagacagatccagtctgaccggctttcatcactagtcttctcacccttcatttgagcaggaaaagctcatttttctagccctggaaggcgccagggacgctacacggaactcacccagaacaacgccgtatggacccctctgcaggtagcacggaaaagacaggcg

SEQ ID NO: 77 - protein, 47 aa, Homo sapiens
GKWCHACAELPEPASTTSNPLSELPCCCMGWQCPHSAEENLCYTAQW

SEQ ID NO: 78 - DNA, 404 bp, Homo sapiens
cgggaaatggtgccacgcatgcgcagaacttcccgagccagcatccaccacatcaaacccactgagtgagctcccttgttgttgcatgggatggcaatgtccacatagcgcagaggagaatctgtgttacacagcgcaatggtaggtaggttaacataagatgcctccgtgagaggctggtggtcagccctggggtcagtaaccacaagaagccgtggctcccggaaggctgcctggatctggttagtgaaggttccaggagtgaagcggccagcaattggagtggctccagtggcagcagcaaacttcagcacagccctctggccagtattcctggaggatataacactgacatcagcagggttttcaatggcaacaattgcacgagctgccagcagaagctt

SEQ ID NO: 79 - DNA, 5001 bp, Homo sapiens
taatcacattgaaaaaaagtttatttaaaaaagttctagcagcaagaacagtaacaaaagaaaaggagagacaaacaaaacaagagggaggttgtatcccatggaacttgtctcctttcttaatcgagttctttatatcgagcaaacatgactcttcgaacagcatctcggtaagtctcgttggactgtcttttgctggttcttcctgaggctccaacactggggcccctcatccggccttcagcctgtaaaacaaagacaaactatgaagtgggccctatagattaattagcctcagcgctggtctttgaccacaggcatttctggactagtaagaagcagtatataggataattttttttttttttttagatggagtcatgctctgtcgcccgggctagagtgcagtggtgcgatctgggctcactgcaagtctgcctccccggttcaagcaattcttgtgcctcagcctctgagtagctgggactacaggcgcctgccaccacgcccggctaatttttgtatttttagtagagacagggtttcgccatgttggccaggctggtctcgaactcctggcctcaagtgatccgcccaccttggcctcccaaagtgttgggattacagatgtgagccaccacacctggcagaataattttttttttgagacagagtctcgctctgtcacccaggctggagtgcagtggcacgatcaagatcttggctcactgcaacctctgcctccccggtacaagcgattcttctgcctcagcctcctgagtagctgtgattacaggcgcgcgccactgcacccagctaatttttgtatttttagtagagacaggggtttcatcatattggtcaggctggtctcgaactcctgaccttgtgatccacccacctcggcctcccaaagtgctgggattacaagcataagccactgcgcccggaccctggcaggataatttttttaaagaatcttttggccgggcacagtggctcacgcctgtaatcccaacactttgggaggctgaggtgggcggatcacttgaggccaggagttcgagaccagcctggccaacatggcaaaactctgtctctactaaaaacacaaaaattagctgggtatggtggtatgcacctgtaatcccagctactcaggaggttggggtgggagaattgcttgaacctgggaggaggaggttgcagtgagctgagatcgcaccactgcattccagcctgggcgacagagcaagactctgtctcaaaaaaaaaaaaagaaaagaatctttcattattaaacaaatacccaagatgaattaaggtgggtggtgatgctatgattgcaagatacaggctttaataccagagatatgagaaactcggtaggagagaaataagccaatctcttatggttggcattttcagaggagaagcaagccaacacaaaatgggtaatgtttcctcttatcaccttttctagaggctcttgaaatatgaggggcaaagtccttcatgggaaccagaacaacatgctccttaaattttagccaccaaagacttccgctgacagtccctctcagagatgcctctgtacctttcattgctgattccctggttcttttgcccaaatgattcttcagtctcaaactgcctcagatactgctctactatttactccatgtaaaagtatgatgaaatccattccaactccttccttctcttcctaactccaaactgcaaagggcccagcctgccaagctataacccagtattcaaggctaaagtgttcaactctcatgtcctatcccagcagtagagaaagcccagcaaaatggcaaagacatcactctaggccagaaagtgaaagttaattacattccaggttcatgtcaccccccaaaagatggaaaccattttacctcctctgatgaagccaatccaggatggccatatcccaggcctgtccctttcctccagccagtagcctgttggacacagcctcctttgctgctagtgtcgatatcttctttatcaacaagtttacgatcactgcaaaagaagcagacaccatcacttcctttttttttttttttgagacggagtttcactctttttgcccaggctggagtgcaatggctcgaactcagcttactgcaacctctgcctcctgggttcaggcgattctcctgcctcagcctcccgattagctgggattgcaggtgcccgccaccacacctggctaatttttaatttttttttttttcagtagagacagagtttcactgtgttggccaggctgctctcaaactcctgacctcaggtaatccgcatgccttggcctcccaaagtgctggattacaggcatgagccaccatgcccaacctgacaccatcattttgatgaagacctgcttataagcctaatataaaatctctgtcctcttgattcatatctgcctcttattaagtaagtatatttaacagtacctctgagtgcctgatagctatgttttgttgctgggtacacagactactaagatatggccctgttcacaagaaaggtctcaaacattatgtcaagagatgcttatagaacattgaagagagctcaaccatggatgtagcaacagtagatgtctgctatagaaatacagtagaaggaatcactttgggaggctgaggtgggcagatcacctgaggttgggagttcgtgaccagcctgaccaacatggagaaaccctgtctctactaaaaatacaaaattagccgggtgtggtggcgcatgcctgtaatcctagctactcgggaggctgaggcaggagaatcgcttgaacccaggaggtggaggttgtggttagctgatatcgcgccattgcactccagcctgggcaacaagagtgaaactctgtttcaaaaaaaaaaaagaaatgcagtagaaggaacgaaaactctgaaggggcatcaccaaaaatgacaggaatttcaacaggaagatacatctgaattggatctcgaaataaggagtttgtgtaagagaaaaggaggacacaagcaaggagacacaaaagacaatttgtccaagagagtagtagtagaaactgacaaaggtaaggctgcttggtggccgggtgcagtgactcacgcctgtaatcccagcactttgggaggccaaggcgggtggatcacctgaggtcaggagttcgagaccaccctgaccaacaggtgaaacccctctctactaaaaatacaaacattagcccatagtcccagctactggggaggctgaggcaggagaatcgcttgaacctgggaggcggaggttgcagtgagccaagatcgtgccattgcactccagcctgggcgacagaatgagactgtctcaaaacaaaaggaaaaaaaaaaaaggctgcatggggtcagactgtaaaaaaccttgaacttattaagctaaatcatgtctctgaacctcatccttctggcaaaagttggagctcaggcctccctgtaggcaaggtatacggtttgtaaaaagacaagttataagagtagatttctctttacaaaagtcattttggtggcagggtagagaatggactgaagaacagtgggagcaaagactgggagtaggagtccagtgtggaggtggaatagaatgggagatgctgtggtttaaatgtgtccctcaaagtacatatgttggaaacataatccccaatgcaacagtattgagaggtgaaacttcaggaagtgattaggtcatgagagctctgccctcacaaatggattaatgctgttattgcaggagtgggttctcctgataaaatgattagtctggtttctttctcccaaccccctcttttgcctaagctctcttgttcttttactttccaccatgaggtgacacagcaagaaggccctcaccagatgcagctcctcaatcttggacctcccagcctccagaagcatgagccaaataaatttctgttcattataaattacccagtctgtggtattcagttatggtatcataaaacagactaagacagggggcaagacttaattagggatttggtcactaattggacacaggggataaaggaggaggatggatcagaccatgtagacagtgatgctatgatgctgctactctgaaataataatacaggagatttggacttgttagactgtggtggtggttgaggccatgggtaccttagaaagcccaggtcaagagggtagagcagcaagaccaaagaagcactgagagaagagtcctagagaacacctggatttatccagaaaaggaagaggagcagaaaaggtgactggaaaggagaagagccaggagaatgtggtataaagggtgctgagaataaagaagggtctctttccgcaccccacctaagccttgtcccagaagactcaaggccaaggtgtccttcctcctgatggctgactccttcacctagtcctaatccactgtatttcaaattgcagagaatgacccaatggaatgtcacactaaatggtcccactgtaacaactcataaaaatgaagagagactagacttgaataaaaatatcagggttcactgtacctggtaagaaagtattttagatgaaaactttttcagttgtgcacacacacactttattattgggccatgatacaaaaatacttctcttattgtgggttggatcaaataagctggaatgccagagccccaaaacctgtccagcctcctcaagggccttgctctgtcaattatctccctttccttctgctcatgcatccacttgttacccacttttccttttcttttcttttcttttttttttttgatttggagtctcgctctattacccaggctagagtacagtggtgcaatcttggctcactgcaacctccacctcccatgttcaagcgattctcctgccccagtctcccgagtagctgggattacaggtacccaccaccacgcctggctaatttttttat

SEQ ID NO: 80 - protein, 22 aa, Homo sapiens
LRGITKNDRNFNRKIHLNWISK

SEQ ID NO: 81 - DNA, 453 bp, Homo sapiens
ctctgaggggcatcaccaaaaatgacaggaatttcaacaggaagatacatctgaattggatctcgaaataaggagtttgtgtaagagaaaaggaggacacaagcaaggagacacaaaagacaatttgtccaagagagtagtagtagaaactgacaaaggtaaggctgcttggtggccgggtgcagtgactcacgcctgtaatcccagcactttgggaggccaaggcgggtggatcacctgaggtcaggagttcgagaccaccctgaccaacaggtgaaacccctctctactaaaaatacaaacattagcccatagtcccagctactggggaggctgaggcaggagaatcgcttgaacctgggaggcggaggttgcagtgagccaagatcgtgccattgcactccagcctgggcgacagaatgagactgtctcaaaacaaaaggaaaaaaaaaa

SEQ ID NO: 82 - protein, 76 aa, Homo sapiens
PASASILAGVPMYRNEFTAWRRMSVVYGIGTWSVLGSLLYYSRTMAKSSVDQKDGSASEVPSELSERPSLRPHSSN

SEQ ID NO: 83 - DNA, 228 bp, Homo sapiens
ccggcgagcgcgagcaattctggcgggcgtgccgatggtatcgaacgaattaccgcgtggtatcgccgcatgagcgtggtgtatggcattggcacctggagcgtgctgggcagcctgctgtattatagccgcaccatggcgaaaagcagcgtggatcagaaagatggcagcgcggaagtgccgagcgaactgagcgaacgcccgagcctgcgcccgcatagcagcacc

(19) United States
(12) Patent Application Publication
(10) Pub. No.: US 2013/0130355 A1
(43) Pub. Date: May 23, 2013

(54) METHOD AND SYSTEM OF PARTICLE-PHAGE EPITOPE COMPLEX

(71) Applicants: Jeanne OHRNBERGER, Northville, MI (US); Stephen A. SPINDLER, Ypsilanti, MI (US)

(72) Inventors: Jeanne OHRNBERGER, Northville, MI (US); Stephen A. SPINDLER, Ypsilanti, MI (US)

(73) Assignee: ARMUNE BIOSCIENCES, INC., Kalamazoo, MI (US)

(21) Appl. No.: 13/631,389

(22) Filed: Sep. 28, 2012

Related U.S. Application Data
(60) Provisional application No. 61/540,360, filed on Sep. 28, 2011.

(51) Int. Cl. C12N 7/00

(52) U.S. Cl. CPC C12N 7/00
USPC 435/235.1

(57) ABSTRACT

The present disclosure provides compositions and methods for using phage epitopes to profile the immune response. The phage epitopes can be used to detect one or more antibodies from a sample. Furthermore, the present disclosure provides methods and compositions for detecting a cancer based on the detection of one or more antibodies. In one embodiment, the antibody is an autoantibody. The present disclosure also provides methods of producing antibody detecting complexes.

### CROSS-REFERENCE

This application claims the benefit of U.S. Provisional Application No. 61/540,360, filed Sep. 28, 2011, which application is incorporated herein by reference in its entirety.

### BACKGROUND OF THE INVENTION

Early detection, prognostic prediction, and monitoring of a disease or condition are desirable for therapeutic decisions. Identification of a disease or condition at the earliest stage can provide a higher probability for effective treatment than identifying the disease or condition at a later stage. Thus, assessing one or more biomarkers in a sample from a subject, such as with improved sensitivity, specificity, or accuracy, is advantageous in detecting a disease or condition in the subject.

A novel system and method in detecting one or more biomarkers, such as a plurality of biomarkers, is provided herein. The system and method disclosed herein can be used to detect a condition or disease.

### SUMMARY OF THE INVENTION

Disclosed here are methods of producing an antibody detecting complex, the methods comprising: (a) providing a polypeptide probe, wherein the probe is capable of being specifically bound by an antibody, and the probe is present on a phage; and (b) coupling the phage to a particle, wherein the coupling occurs in a buffer, thereby producing the antibody detecting complex. In one embodiment, the coupling comprises a linker, wherein the linker covalently couples the particle to the phage. In one embodiment, the antibody detecting complex is stable for a longer period of time than an antibody complex produced without the buffer. In one embodiment, the buffer maintains a pH of between about 4 and 6. In one embodiment, the buffer maintains a pH of about 5. In one embodiment, the buffer comprises MES (2-(N-morpholino) ethanesulfonic acid). In one embodiment, the antibody is an autoantibody. In one embodiment, the autoantibody is cancer autoantibody. In one embodiment, the cancer autoantibody is prostate cancer autoantibody. In one embodiment, the polypeptide probe comprises a full length or fragment of a protein encoded by alpha-2 glycoprotein1 (AZGP1), DCHS1 (protocadherin-16 precursor), Homo sapiens family with sequence similarity 53, member B (FAM53B), mitogen-activated protein kinase kinase kinase 9 (MAPKKK9), Deaminase Domain, ADP-ribosylation factor 6 (ARF6), Centrosomal Protein 164 kDa (CEP 164), ADAM metallopeptidase domain 9 (ADAMS), splicing factor, arginine/serine-rich 14 (SFRS14), cytochrome c oxidase subunit Va (Cox5a), TIMP metallopeptidase inhibitor 2 (TIMP2), breast cancer metastasis-suppressor 1-like (BRMSL1), RP3-323M22 (Nucleolin), Homo sapiens chromosome 3 genomic contigs (NKX3-1), casein kinase 2, alpha prime polypeptide (CSNK2A2), Desmocollin 3 (DSC3), ribosomal protein L34 (RPL34), Chromosome 3 UTR region ropporin/RhoEGF (3′ UTR Ropporin), aurora kinase A interacting protein 1 (AURKAIP1), 5′-UTR BMI1, ribosomal protein SA (LAMR1), Contig GRCh37.p2 (1371451::1372008), Prostate Specific Antigen (PSA), ribosomal protein SA (RPSA) (minus strand), RNA Binding Protein 6 (RBM6) (minus strand), or hypothetical protein XP_{—}353238. In one embodiment, the polypeptide probe comprises a full length or protein fragment encoded by SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65 SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 76, or SEQ ID NO: 79. In one embodiment, the polypeptide probe comprises a full length or protein fragment encoded by SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 75, SEQ ID NO: 78, SEQ ID NO: 81, or SEQ ID NO: 83. In one embodiment, the polypeptide probe comprises SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 74, SEQ ID NO: 77, SEQ ID NO: 80, or SEQ ID NO: 82, or a fragment thereof. In one embodiment, the phage is a t7 phage. In one embodiment, the particle is a microsphere. In one embodiment, the microsphere comprises polystyrene. In one embodiment, the microsphere comprises identification information. In one embodiment, the identification information comprises a fluorescent signal. In one embodiment, the identification information comprises microsphere size. In one embodiment, the microsphere is magnetic. In one embodiment, the antibody detecting complex is used to detect autoantibodies. In one embodiment, the antibody detecting complex is used to detect a disease or condition. In one embodiment, the disease or condition is a cardiovascular condition, autoimmune condition, inflammatory condition, infectious disease, or neurological disease. In one embodiment, the disease or condition is cancer. In one embodiment, the cancer is prostate cancer, breast cancer, a carcinoma, a sarcoma, a lymphoma, a germ cell tumor, or a blastoma.

Also disclosed herein are compositions for producing an antibody detecting complex comprising: (a) a polypeptide probe, wherein the probe is capable of being specifically bound by an antibody, and the probe is present on a phage; (b) a particle; and, (c) a buffer, wherein the buffer maintains conditions for coupling the phage to the particle to produce the antibody detecting complex. In one embodiment, the coupling comprises a linker, wherein the linker covalently couples the particle to the phage. In one embodiment, the antibody detecting complex is stable for a longer period of time than an antibody complex produced without the buffer. In one embodiment, the buffer maintains a pH of between about 4 and 6. In one embodiment, the buffer maintains a pH of about 5. In one embodiment, the buffer comprises MES (2-(N-morpholino) ethanesulfonic acid). In one embodiment, the antibody is an autoantibody. In one embodiment, the autoantibody is cancer autoantibody. In one embodiment, the cancer autoantibody is prostate cancer autoantibody. In one embodiment, the polypeptide probe comprises a full length or fragment of a protein encoded by AZGP1, DCHS1, FAM53B, MAPKKK9, Deaminase Domain, ARF6, CEP 164, ADAMS, SFRS14, Cox5a, TIMP2, BRMSL1, Nucleolin, NKX3-1, CSNK2A2, DSC3, RPL34, 3′ UTR Ropporin, AURKAIP1,5′-UTR BMI1, LAMR1, Contig GRCh37.p2 (1371451::1372008), PSA, RPSA (minus strand), RBM6 (minus strand), or hypothetical protein XP_{—}353238. In one embodiment, the polypeptide probe comprises a full length or protein fragment encoded by SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65 SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 76, or SEQ ID NO: 79. In one embodiment, the polypeptide probe comprises a full length or protein fragment encoded by SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 75, SEQ ID NO: 78, SEQ ID NO: 81, or SEQ ID NO: 83. In one embodiment, the polypeptide probe comprises SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 74, SEQ ID NO: 77, SEQ ID NO: 80, or SEQ ID NO: 82, or a fragment thereof. In one embodiment, the phage is a t7 phage. In one embodiment, the particle is a microsphere. In one embodiment, the microsphere comprises polystyrene. In one embodiment, the microsphere comprises identification information. In one embodiment, the identification information comprises a fluorescent signal. In one embodiment, the identification information comprises microsphere size. In one embodiment, the microsphere is magnetic. In one embodiment, the antibody detecting complex is used to detect autoantibodies. In one embodiment, the antibody detecting complex is used to detect a disease or condition. In one embodiment, the disease or condition is a cardiovascular condition, autoimmune condition, inflammatory condition, infectious disease, or neurological disease. In one embodiment, the disease or condition is cancer. In one embodiment, the cancer is prostate cancer, breast cancer, a carcinoma, a sarcoma, a lymphoma, a germ cell tumor, or a blastoma.

Also disclosed herein are kits for producing an antibody detecting complex comprising: (a) a polypeptide probe, wherein the probe is capable of being specifically bound by an antibody, and the probe is present on a phage; (b) a particle; and, (c) a buffer, wherein the buffer maintains conditions for coupling the phage to the particle to produce an antibody detecting complex. Some embodiments further comprise: (d) a linker, wherein the linker covalently couples the particle to the phage. Some embodiments further comprise: (e) instructions for producing the antibody detecting complex.

In another aspect, disclosed herein are antibody detecting complexes comprising: (a) a polypeptide probe, wherein the probe is capable of being specifically bound by an antibody, and the probe is present on a phage; and, (b) a particle, wherein the particle is covalently bound to the phage. Also disclosed herein are antibody detecting complexes comprising: (a) a polypeptide probe, wherein the probe is capable of being specifically bound by an antibody, and the probe is present on a phage; (b) a particle; and, (c) a linker, wherein the linker covalently couples the particle to the phage. In one embodiment, the antibody is an autoantibody. In one embodiment, the autoantibody is cancer autoantibody. In one embodiment, the cancer autoantibody is prostate cancer autoantibody. In one embodiment, the polypeptide probe comprises a full length or fragment of a protein encoded by AZGP1, DCHS1, FAM53B, MAPKKK9, Deaminase Domain, ARF6, CEP 164, ADAMS, SFRS14, Cox5a, TIMP2, BRMSL1, Nucleolin, NKX3-1, CSNK2A2, DSC3, RPL34, 3′ UTR Ropporin, AURKAIP1,5′-UTR BMI1, LAMR1, Contig GRCh37.p2 (1371451::1372008), PSA, RPSA (minus strand), RBM6 (minus strand), or hypothetical protein XP_{—}353238. In one embodiment, the polypeptide probe comprises a full length or protein fragment encoded by SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65 SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 76, or SEQ ID NO: 79. In one embodiment, the polypeptide probe comprises a full length or protein fragment encoded by SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 75, SEQ ID NO: 78, SEQ ID NO: 81, or SEQ ID NO: 83. In one embodiment, the polypeptide probe comprises SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 74, SEQ ID NO: 77, SEQ ID NO: 80, or SEQ ID NO: 82, or a fragment thereof. In one embodiment, the phage is a t7 phage. In one embodiment, the particle is a microsphere. In one embodiment, the microsphere comprises polystyrene. In one embodiment, the microsphere comprises identification information. In one embodiment, the identification information comprises a fluorescent signal. In one embodiment, the identification information comprises microsphere size. In one embodiment, the antibody detecting complex further comprises a linker between the phage and the particle. In one embodiment, the antibody detecting complex further comprises a linker between the phage and the polypeptide probe. In one embodiment, the linker covalently joins the phage and the particle. In one embodiment, the linker non-covalently joins the phage and the particle. In one embodiment, the linker covalently joins the phage and the polypeptide probe. In one embodiment, the linker non-covalently joins the phage and the polypeptide probe. In one embodiment, the antibody detecting complex is used to detect autoantibodies. In one embodiment, the antibody detecting complex is used to detect a disease or condition. In one embodiment, the disease or condition is a cardiovascular condition, autoimmune condition, inflammatory condition, infectious disease, or neurological disease. In one embodiment, the disease or condition is cancer. In one embodiment, the cancer is prostate cancer, breast cancer, a carcinoma, a sarcoma, a lymphoma, a germ cell tumor, or a blastoma.

Also disclosed herein are antibody profiling panels comprising a plurality of antibody detecting complexes, wherein each of the antibody detecting complexes comprise: (a) a polypeptide probe, wherein the probe is capable of being specifically bound by an antibody, and the probe is present on a phage; and, (b) a particle, wherein the particle is covalently coupled to the phage. Also disclosed are antibody profiling panels comprising a plurality of antibody detecting complexes, wherein each of the antibody detecting complexes comprise: (a) a polypeptide probe, wherein the probe is capable of being specifically bound by an antibody, and the probe is present on a phage; (b) a particle; and, (c) a linker, wherein the linker covalently couples the particle to the phage. In one embodiment, the plurality of antibody detecting complexes comprises at least 5 polypeptide probes. In one embodiment, the plurality of antibody detecting complexes comprises at least 10 polypeptide probes. In one embodiment, the antibody is an autoantibody. In one embodiment, the autoantibody is cancer autoantibody. In one embodiment, the cancer autoantibody is prostate cancer autoantibody. In one embodiment, the polypeptide probe comprises a full length or fragment of a protein encoded by AZGP1, DCHS1, FAM53B, MAPKKK9, Deaminase Domain, ARF6, CEP 164, ADAMS, SFRS14, Cox5a, TIMP2, BRMSL1, Nucleolin, NKX3-1, CSNK2A2, DSC3, RPL34, 3′ UTR Ropporin, AURKAIP1,5′-UTR BMI1, LAMR1, Contig GRCh37.p2 (1371451::1372008), PSA, RPSA (minus strand), RBM6 (minus strand), or hypothetical protein XP_{—}353238. In one embodiment, the polypeptide probe comprises a full length or protein fragment encoded by SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65 SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 76, or SEQ ID NO: 79. In one embodiment, the polypeptide probe comprises a full length or protein fragment encoded by SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 75, SEQ ID NO: 78, SEQ ID NO: 81, or SEQ ID NO: 83. In one embodiment, the polypeptide probe comprises SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 74, SEQ ID NO: 77, SEQ ID NO: 80, or SEQ ID NO: 82, or a fragment thereof. In one embodiment, the phage is a t7 phage. In one embodiment, the particle is a microsphere. In one embodiment, the microsphere comprises polystyrene. In one embodiment, the microsphere comprises identification information. In one embodiment, the identification information comprises a fluorescent signal. In one embodiment, the identification information comprises microsphere size. In one embodiment, each of the antibody detecting complexes further comprises a linker between the phage and the particle. In one embodiment, each of the antibody detecting complexes further comprises a linker between the phage and the polypeptide probe. In one embodiment, the linker covalently joins the phage and the particle. In one embodiment, the linker non-covalently joins the phage and the particle. In one embodiment, the linker covalently joins the phage and the polypeptide probe. In one embodiment, the linker non-covalently joins the phage and the polypeptide probe. In one embodiment, the panel is used to detect autoantibodies. In one embodiment, the panel is used to detect a disease or condition. In one embodiment, the disease or condition is a cardiovascular condition, autoimmune condition, inflammatory condition, infectious disease, or neurological disease. In one embodiment, the disease or condition is cancer. In one embodiment, the cancer is prostate cancer, breast cancer, a carcinoma, a sarcoma, a lymphoma, a germ cell tumor, or a blastoma.

Also disclosed herein are methods for detecting a disease or condition comprising: (a) contacting a sample from a subject with an antibody detecting complex, wherein the antibody detecting complex comprises: (i) a polypeptide probe, wherein the probe is capable of being specifically bound by an antibody, and the probe is present on a phage, and (ii) a particle, wherein the particle is covalently coupled to the phage; (b) detecting a presence or level of an antibody bound to the antibody detecting complex; and (c) detecting the disease or condition based on the presence or level of the antibody. Also disclosed are methods for detecting a disease or condition comprising: (a) contacting a sample from a subject with an antibody detecting complex, wherein the antibody detecting complex comprises: (i) a polypeptide probe, wherein the probe is capable of being specifically bound by an antibody, and the probe is present on a phage, (ii) a particle, and (iii) a linker, wherein the linker covalently couples the particle to the phage; (b) detecting a presence or level of an antibody bound to the antibody detecting complex; and (c) detecting the disease or condition based on the presence or level of the antibody. In one embodiment, the disease or condition is a cardiovascular condition, autoimmune condition, inflammatory condition, infectious disease, or neurological disease. In one embodiment, the antibody detecting complex characterizes or screens the disease or condition with at least 80% specificity. In one embodiment, the antibody is an autoantibody. In one embodiment, the autoantibody is a cancer autoantibody. In one embodiment, the cancer autoantibody is a prostate cancer autoantibody. In one embodiment, the disease or condition is cancer. In one embodiment, the antibody detecting complex detects the cancer with at least 80% specificity. In one embodiment, the cancer is prostate cancer, breast cancer, a carcinoma, a sarcoma, a lymphoma, a germ cell tumor, or a blastoma. In one embodiment, the cancer is prostate cancer. In one embodiment, the polypeptide probe comprises a full length or fragment of a protein encoded by AZGP1, DCHS1, FAM53B, MAPKKK9, Deaminase Domain, ARF6, CEP 164, ADAMS, SFRS14, Cox5a, TIMP2, BRMSL1, Nucleolin, NKX3-1, CSNK2A2, DSC3, RPL34, 3′ UTR Ropporin, AURKAIP1,5′-UTR BMI1, LAMR1, Contig GRCh37.p2 (1371451::1372008), PSA, RPSA (minus strand), RBM6 (minus strand), or hypothetical protein XP_{—}353238. In one embodiment, the polypeptide probe comprises a full length or protein fragment encoded by SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65 SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 76, or SEQ ID NO: 79. In one embodiment, the polypeptide probe comprises a full length or protein fragment encoded by SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 75, SEQ ID NO: 78, SEQ ID NO: 81, or SEQ ID NO: 83. In one embodiment, the polypeptide probe comprises SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 74, SEQ ID NO: 77, SEQ ID NO: 80, or SEQ ID NO: 82, or a fragment thereof. In one embodiment, the phage is a t7 phage. In one embodiment, the particle is a microsphere. In one embodiment, the microsphere comprises polystyrene. In one embodiment, the microsphere comprises identification information. In one embodiment, the identification information comprises a fluorescent signal. In one embodiment, the identification information comprises microsphere size. In one embodiment, the antibody detecting complex further comprises a linker between the phage and the particle. In one embodiment, the antibody detecting complex further comprises a linker between the phage and the polypeptide probe. In one embodiment, the linker covalently joins the phage and the particle. In one embodiment, the linker non-covalently joins the phage and the particle. In one embodiment, the linker covalently joins the phage and the polypeptide probe. In one embodiment, the linker non-covalently joins the phage and the polypeptide probe.

Also disclosed herein are methods for detecting a disease or condition comprising: (a) contacting a sample from a subject with an antibody profiling panel, wherein the panel comprises a plurality of antibody detecting complexes, wherein each of the antibody detecting complex comprises: (i) a polypeptide probe, wherein the probe is capable of being specifically bound by an antibody, and the probe is present on a phage, and, (ii) a particle, wherein the particle is covalently coupled to the phage; (b) detecting a presence or level of a plurality of antibodies bound to the plurality of antibody detecting complexes; and (c) detecting the disease or condition based on the presence or level of the plurality of antibodies. Also disclosed are methods for detecting a disease or condition comprising: (a) contacting a sample from a subject with an antibody profiling panel, wherein the panel comprises a plurality of antibody detecting complexes, wherein each of the antibody detecting complex comprises: (i) a polypeptide probe, wherein the probe is capable of being specifically bound by an antibody, and the probe is present on a phage, (ii) a particle, and, (iii) a linker, wherein the linker covalently couples the particle to the phage; (b) detecting a presence or level of a plurality of antibodies bound to the plurality of antibody detecting complexes; and (c) detecting the disease or condition based on the presence or level of the plurality of antibodies. In one embodiment, the plurality of antibody detecting complexes comprises at least 5 polypeptide probes. In one embodiment, the plurality of antibody detecting complexes comprises at least 10 polypeptide probes. In one embodiment, the disease or condition is a cardiovascular condition, autoimmune condition, inflammatory condition, infectious disease, or neurological disease. In one embodiment, the antibody profiling panel detects the disease or condition with at least 80% specificity. In one embodiment, the antibody is an autoantibody. In one embodiment, the autoantibody is a cancer autoantibody. In one embodiment, the cancer autoantibody is a prostate cancer autoantibody. In one embodiment, the disease or condition is cancer. In one embodiment, the antibody profiling panel detects the cancer with at least 80% specificity. In one embodiment, the cancer is prostate cancer, breast cancer, a carcinoma, a sarcoma, a lymphoma, a germ cell tumor, or a blastoma. In one embodiment, the cancer is prostate cancer. In one embodiment, the polypeptide probe comprises a full length or fragment of a protein encoded by AZGP1, DCHS1, FAM53B, MAPKKK9, Deaminase Domain, ARF6, CEP 164, ADAMS, SFRS14, Cox5a, TIMP2, BRMSL1, Nucleolin, NKX3-1, CSNK2A2, DSC3, RPL34, 3′ UTR Ropporin, AURKAIP1,5′-UTR BMI1, LAMR1, Contig GRCh37.p2 (1371451::1372008), PSA, RPSA (minus strand), RBM6 (minus strand), or hypothetical protein XP_{—}353238. In one embodiment, the polypeptide probe comprises a full length or protein fragment encoded by SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65 SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 76, or SEQ ID NO: 79. In one embodiment, the polypeptide probe comprises a full length or protein fragment encoded by SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 75, SEQ ID NO: 78, SEQ ID NO: 81, or SEQ ID NO: 83. In one embodiment, the polypeptide probe comprises SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 74, SEQ ID NO: 77, SEQ ID NO: 80, or SEQ ID NO: 82, or a fragment thereof. In one embodiment, the phage is a t7 phage. In one embodiment, the particle is a microsphere. In one embodiment, the microsphere comprises polystyrene. In one embodiment, the microsphere comprises identification information. In one embodiment, the identification information comprises a fluorescent signal. In one embodiment, the identification information comprises microsphere size. In one embodiment, the antibody detecting complex further comprises a linker between the phage and the particle. In one embodiment, the antibody detecting complex further comprises a linker between the phage and the polypeptide probe. In one embodiment, the linker covalently joins the phage and the particle. In one embodiment, the linker non-covalently joins the phage and the particle. In one embodiment, the linker covalently joins the phage and the polypeptide probe. In one embodiment, the linker non-covalently joins the phage and the polypeptide probe.

Also disclosed herein are cancer autoantibody detecting complexes comprising: (a) a polypeptide probe, wherein the probe is capable of being specifically bound by a cancer autoantibody, and the probe is present on a phage; and, (b) a particle, wherein the particle is covalently bound to the phage. Also disclosed are cancer autoantibody detecting complexes comprising: (a) a polypeptide probe, wherein the probe is capable of being specifically bound by a cancer autoantibody, and the probe is present on a phage; (b) a particle; and, (c) a linker, wherein the linker covalently couples the particle to the phage. Also disclosed are prostate cancer autoantibody detecting complexes comprising: (a) a polypeptide probe, wherein the probe is capable of being specifically bound by a prostate cancer autoantibody, and the probe is present on a phage; and, (b) a particle, wherein the particle is covalently bound to the phage. Also disclosed are prostate cancer autoantibody detecting complexes comprising: (a) a polypeptide probe, wherein the probe is capable of being specifically bound by a prostate cancer autoantibody, and the probe is present on a phage; (b) a particle; and, (c) a linker, wherein the linker covalently couples the particle to the phage. Also disclosed are cancer autoantibody profiling panels comprising a plurality of cancer autoantibody detecting complexes, wherein each of the cancer autoantibody detecting complex comprises: (a) a polypeptide probe, wherein the probe is capable of being specifically bound by a cancer autoantibody, and the probe is present on a phage; and, (b) a particle, wherein the particle is covalently bound to the phage. Also disclosed are cancer autoantibody profiling panel comprising a plurality of cancer autoantibody detecting complexes, wherein each of the cancer autoantibody detecting complex comprises: (a) a polypeptide probe, wherein the probe is capable of being specifically bound by a cancer autoantibody, and the probe is present on a phage; (b) a particle; and, (c) a linker, wherein the linker covalently couples the particle to the phage. Also disclosed are prostate cancer autoantibody profiling panels comprising a plurality of prostate cancer autoantibody detecting complexes, wherein each of the prostate cancer autoantibody detecting complex comprises: (a) a polypeptide probe, wherein the probe is capable of being specifically bound by a prostate cancer autoantibody, and the probe is present on a phage; and, (b) a particle, wherein the particle is covalently bound to the phage. Also disclosed are prostate cancer autoantibody profiling panels comprising a plurality of prostate cancer autoantibody detecting complexes, wherein each of the prostate cancer autoantibody detecting complex comprises: (a) a polypeptide probe, wherein the probe is capable of being specifically bound by a prostate cancer autoantibody, and the probe is present on a phage; (b) a particle; and, (c) a linker, wherein the linker covalently couples the particle to the phage.

Also disclosed herein are methods for detecting cancer in a subject, comprising: (a) contacting a sample from the subject with a cancer autoantibody detecting complex, wherein the cancer autoantibody complex comprises: (i) a polypeptide probe, wherein the probe is capable of being specifically bound by a cancer autoantibody, and the probe is present on a phage, and, (ii) a particle, wherein the particle is covalently coupled to the phage; (b) detecting a presence or level of a cancer autoantibody bound to the cancer autoantibody detecting complex; and, (c) detecting the cancer based on the presence or level of the cancer autoantibody. Also disclosed are methods for detecting cancer in a subject, comprising: (a) contacting a sample from the subject with a cancer autoantibody detecting complex, wherein the cancer autoantibody complex comprises: (i) a polypeptide probe, wherein the probe is capable of being specifically bound by a cancer autoantibody, and the probe is present on a phage, (ii) a particle, and (iii) a linker, wherein the linker covalently couples the particle to the phage; (b) detecting a presence or level of a cancer autoantibody bound to the cancer autoantibody detecting complex; and, (c) detecting the cancer based on the presence or level of the cancer autoantibody. Also disclosed are methods for detecting prostate cancer in a subject, comprising: (a) contacting a sample from the subject with a prostate cancer autoantibody detecting complex, wherein the prostate cancer autoantibody complex comprises: (i) a polypeptide probe, wherein the probe is capable of being specifically bound by a prostate cancer autoantibody, and the probe is present on a phage, and (ii) a particle, wherein the particle is covalently coupled to the phage; (b) detecting a presence or level of a prostate cancer autoantibody bound to the prostate cancer autoantibody detecting complex; and, (c) detecting the prostate cancer based on the presence or level of the prostate cancer autoantibody. Also disclosed are methods for detecting prostate cancer in a subject, comprising: (a) contacting a sample from the subject with a prostate cancer autoantibody detecting complex, wherein the prostate cancer autoantibody complex comprises: (i) a polypeptide probe, wherein the probe is capable of being specifically bound by a prostate cancer autoantibody, and the probe is present on a phage, (ii) a particle, and (iii) a linker, wherein the linker covalently couples the particle to the phage; (b) detecting a presence or level of a prostate cancer autoantibody bound to the prostate cancer autoantibody detecting complex; and, (c) detecting the prostate cancer based on the presence or level of the prostate cancer autoantibody. Also disclosed are methods for detecting cancer in a subject, comprising: (a) contacting a sample from the subject with a cancer autoantibody detecting panel comprising a plurality of cancer autoantibody detecting complexes, wherein each of the cancer autoantibody detecting complexes comprise: (i) a polypeptide probe, wherein the probe is capable of being specifically bound by a cancer autoantibody, and the probe is present on a phage, and (ii) a particle, wherein the particle is covalently coupled to the phage; (b) detecting a presence or level of cancer autoantibodies bound to the cancer autoantibody detecting panel; and, (c) detecting the cancer based on the presence or level of the cancer autoantibodies. Also disclosed are methods for detecting cancer in a subject, comprising: (a) contacting a sample from the subject with a cancer autoantibody detecting panel comprising a plurality of cancer autoantibody detecting complexes, wherein each of the cancer autoantibody detecting complexes comprise: (i) a polypeptide probe, wherein the probe is capable of being specifically bound by a cancer autoantibody, and the probe is present on a phage, (ii) a particle, and (iii) a linker, wherein the linker covalently couples the particle to the phage; (b) detecting a presence or level of cancer autoantibodies bound to the cancer autoantibody detecting panel; and, (c) detecting the cancer based on the presence or level of the cancer autoantibodies. Also disclosed are methods for detecting prostate cancer in a subject, comprising: (a) contacting a sample from the subject with a prostate cancer autoantibody detecting panel comprising a plurality of prostate cancer autoantibody detecting complexes, wherein each of the prostate cancer autoantibody detecting complexes comprise: (i) a polypeptide probe, wherein the probe is capable of being specifically bound by a prostate cancer autoantibody, and the probe is present on a phage, and (ii) a particle, wherein the particle is covalently coupled to the phage; (b) detecting a presence or level of prostate cancer autoantibodies bound to the prostate cancer autoantibody detecting panel; and, (c) detecting the prostate cancer based on the presence or level of the prostate cancer autoantibodies. Also disclosed are methods for detecting prostate cancer in a subject, comprising: (a) contacting a sample from the subject with a prostate cancer autoantibody detecting panel comprising a plurality of prostate cancer autoantibody detecting complexes, wherein each of the prostate cancer autoantibody detecting complexes comprise: (i) a polypeptide probe, wherein the probe is capable of being specifically bound by a prostate cancer autoantibody, and the probe is present on a phage, (ii) a particle, and (iii) a linker, wherein the linker covalently couples the particle to the phage; (b) detecting a presence or level of prostate cancer autoantibodies bound to the cancer autoantibody detecting panel; and, (c) detecting the prostate cancer based on the presence or level of the prostate cancer autoantibodies.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates stability of a coupled complex up to 6 months.

FIG. 2 illustrates detection level of biomarkers in a sample with autoantibody detection complex up to 6 months.

FIG. 3 illustrates reproducibility of the coupling of a phage to a particle.

FIG. 4 illustrates detection level of biomarkers in a sample with autoantibody detection complex up to 9 months using A. a single T7 preparation and B. a single 12B2 preparation.

FIG. 5 illustrates detection levels for a single sample screened with 20 different biomarkers.

FIG. 6 illustrates the linearity of biomarker detection for 4 samples screened with a biomarker.

FIG. 7 contains the nucleotide sequence of Homo sapiens alpha-2-glycoprotein 1, zinc-binding (AZGP1), mRNA.

FIG. 8 (A-D) contains the nucleotide sequence of Homo sapiens DCHS1 gene for protocadherin-16 precursor, complete cds, without stop codon, AB384634.1

FIG. 9 (A-B) contains the nucleotide sequence of Homo sapiens family with sequence similarity 53, member B (FAM53B), mRNA.

FIG. 10 (A-B) contains the nucleotide sequence of Homo sapiens mitogen-activated protein kinase kinase kinase 9(MAP3K9), mRNA.

FIG. 11 contains the nucleotide sequence from clone RP1-20N2 on chromosome 6q24. Contains the gene for a novel protein similar to yeast and bacterial cytosine deaminase, NTs 48121-50100 (Deaminase Domain).

FIG. 12 (A-B) contains the nucleotide sequence of Homo sapiens ADP-ribosylation factor 6 (ARF6), mRNA.

FIG. 13 (A-B) contains the nucleotide sequence of Homo sapiens centrosomal protein 164 kDa (CEP164), mRNA, NM_{—}014956.4.

FIG. 14 (A-B) contains the nucleotide sequence of Homo sapiens ADAM metallopeptidase domain 9 (ADAMS), transcript variant 2, non-coding RNA.

FIG. 15 (A-B) contains the nucleotide sequence of Homo sapiens splicing factor, arginine/serine-rich 14 (SFRS14), transcript variant 1, mRNA.

FIG. 16 contains the nucleotide sequence of Homo sapiens cytochrome c oxidase subunit Va (COX5A), nuclear gene encoding mitochondrial protein, mRNA.

FIG. 17 (A-B) contains the nucleotide sequence of Homo sapiens TIMP metallopeptidase inhibitor 2 (TIMP2), mRNA.

FIG. 18 contains the nucleotide sequence of Homo sapiens breast cancer metastasis-suppressor 1-like (BRMS1L), mRNA.

FIG. 19 contains the nucleotide sequence of Homo sapiens nucleolin (NCL), mRNA.

FIG. 20 contains the nucleotide sequence of Homo sapiens 3 BAC RP13-61613 (Roswell Park Cancer Institute Human BAC Library) NTs 22921-24900 (NKX3-1).

FIG. 21 contains the nucleotide sequence of Homo sapiens casein kinase 2, alpha prime polypeptide (CSNK2A2), mRNA.

FIG. 22 contains the nucleotide sequence of Homo sapiens desmocollin 3 (DSC3), RefSeqGene on chromosome 18. NTs 46261-48240.

FIG. 23 contains the nucleotide sequence of Homo sapiens ribosomal protein L34 (RPL34), transcript variant 2, mRNA.

FIG. 24 contains the nucleotide sequence of Homo sapiens 3 BAC RP11-783D3 (Roswell Park Cancer Institute Human BAC Library) NTs 178621-180600 (Chromosome 3 UTR region ropporin/RhoEGF).

FIG. 25 contains the nucleotide sequence of Homo sapiens aurora kinase A interacting protein 1 (AURKAIP1), transcript variant 1, mRNA.

FIG. 26 (A-B) contains the nucleotide sequence of Homo sapiens BMI1 polycomb ring finger oncogene, mRNA (cDNA clone MGC:12685 IMAGE:4138748), complete cds.

FIG. 27 contains the nucleotide sequence of Homo sapiens ribosomal protein SA (RPSA), transcript variant 1, mRNA. Also known as LAMR1.

FIG. 28 contains the nucleotide sequence of Homo sapiens chromosome 19 genomic contig, GRCh37.p2 (1371451::1372008).

FIG. 29 (A-C) contains the nucleotide sequence of Human prostate specific antigen gene, complete cds.

FIG. 30 (A-B) contains the nucleotide sequence of Homo sapiens ribosomal protein SA (RPSA) (minus strand) (gi|224589815:c39454033-39448204 Homo sapiens chromosome 3, GRCh37.p9 Primary Assembly).

FIG. 31 (A-B) contains the nucleotide sequence of Homo sapiens Ribosomal Binding Protein 6 (minus strand) (g|224589815:c50114685-50109685 Homo sapiens chromosome 3, GRCh37.p9 Primary Assembly).

### DETAILED DESCRIPTION OF THE INVENTION

The compositions and methods of the present disclosure relate to a complex for detecting molecules. In one embodiment, the complex comprises a particle and a probe, wherein the probe is displayed or present on a phage. In one embodiment, the phage is linked or coupled to the particle. In one embodiment, the phage is covalently linked to the particle. In another embodiment, the phage is non-covalently linked to the particle. In yet another embodiment, the phage is conjugated to the particle. In one embodiment, a linker is incorporated between the phage and the particle. In one embodiment, the linker covalently couples the phage and particle. In another embodiment, the linker non-covalently couples the particle and the phage. In another embodiment, a linker is incorporated between the phage and the probe. In one embodiment, the linker covalently couples the phage and probe. In another embodiment, the linker non-covalently couples the phage and probe. Detection of a molecule in a sample from a subject can be used to detect a disease or condition in the subject. Various diseases or conditions such as, but not limited to, a cancer, cardiovascular condition, neurological disorder, autoimmune disease, inflammatory condition, or an infectious disease, can be detected by a method or composition described herein.

In one embodiment, the complex is an antibody detecting complex. In one embodiment, the complex detects an autoantibody. In one embodiment, the antibody detecting complex is used to characterize, screen, identify or detect a disease or condition. Various diseases or conditions such as, but not limited to, a cancer, cardiovascular condition, neurological disorder, autoimmune disease, or an infectious disease, can be detected by a method or composition described herein.

A disease or condition can be detected for a subject using a composition or method disclosed herein. In one embodiment, the subject is an individual or patient. In another embodiment, the subject has a pre-existing condition. In one another embodiment, the subject is a cancer patient. In another embodiment, the subject does not have a pre-existing condition. In another embodiment, the subject exhibits no symptom of a disease or condition. In another embodiment, a subject has no detectable symptom. In one embodiment, the individual is an asymptomatic individual. In another embodiment, the individual is a symptomatic individual. In one embodiment, the disease is a cancer, such as prostate cancer. In yet another embodiment, the subject exhibits a symptom of a disease or condition. In one embodiment, the disease is a cancer, such as prostate cancer. The subject can be a mammal, including, but not limited to, humans, non-human primates, rodents, and the like. In one embodiment, the subject is a human.

Detecting a disease or condition (including pre-symptomatic early stage detecting) can include determining the prognosis, diagnosis, or theranosis of a disease or condition, or determining the stage or progression of a disease or condition. In one embodiment, a prognosis is predicting or giving a likelihood of outcome of a disease or condition, such as an extent of malignancy of a cancer, a likelihood of survival, or expected life expectancy. In another embodiment, a prognosis is a prediction or likelihood analysis of a disease or condition progression or recurrence. Detecting a disease or condition can also be screening a subject for a disease or condition, and determining the likelihood or possibility a subject has, or will develop, a disease or condition.

In one embodiment, a theranosis is a therapy selected based on an outcome of determining a binding of one or more antibodies from a sample from a subject to a polypeptide probe as described herein. In one embodiment, a theranosis is identifying an appropriate treatment or treatment efficacy for a cancer. In one embodiment, a theranosis is modifying a treatment. In another embodiment, a theranosis is selecting a treatment regimen. In yet another embodiment, a theranosis is discontinuing or not selecting a particular treatment regimen.

In one embodiment, detecting a disease is detecting a cancer, such as determining the prognosis, diagnosis, or theranosis of a cancer. In yet another embodiment, detecting a cancer is detecting the cancer, such as pre-symptomatic early stage detecting. In one embodiment, detecting a cancer is determining the stage or progression of the cancer, such as early-stage, late-stage or advanced stage of cancer. In another embodiment, detecting a cancer is determining the progression, recurrence, metastatic spread or relapse or the cancer. In one embodiment, the diagnosis is prediction or likelihood an individual or subject has a disease or condition, such as prostate cancer.

A disease or condition can be detected by determining a presence or absence, or level, of one or more antibodies in a sample. In one embodiment, a sample is obtained from a subject. In another embodiment, a sample is a biological fluid. The biological fluid can be, but not limited to, peripheral blood, sera, or plasma. The sample can be ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, or bronchopulmonary aspirates.

Phage-Particle Complex

The compositions and methods of the present disclosure relate to a complex for detecting a molecule, such as a biological molecule or molecule in a sample from a subject. In one embodiment, the complex comprises a particle and a probe, wherein the probe is displayed or present on a phage. In one embodiment, a phage is bound, linked, or coupled to a particle to form a phage-particle complex. In one embodiment, a linker is incorporated between the phage and the particle. In one embodiment, the linker covalently couples the phage and particle. In another embodiment, the linker non-covalently couples the particle and the phage. In another embodiment, a linker is incorporated between the phage and the probe. In one embodiment, the linker covalently couples the phage and probe. In another embodiment, the linker non-covalently couples the phage and probe.

In one embodiment, a phage-particle complex is used to detect a biological molecule, including, but not limited, to an antibody, a ligand, a nucleic acid, or a biological molecule that is capable of forming a non-covalent bond with a probe. In one embodiment, the probe is a polypeptide. In another embodiment the probe is an antigen. In another embodiment, the probe is a receptor or a portion of a receptor. In another embodiment, the probe is a ligand. In another embodiment the probe is a nucleic acid. In another embodiment the probe is a bait molecule that interacts with a specific target molecule (such as a protein or nucleic acid molecule). In another embodiment, the nature of probe decides the biological molecule that can be identified by the probe. For example, if a probe is an antigen, the identifiable biological molecule can be an antibody raised against the antigen. If a probe is a receptor or a portion of a receptor, the identifiable biological molecule can be a ligand that naturally binds to the receptor. If a probe is nucleic acid, the identifiable biological molecule can be a nucleic acid complementary to the probe.

In one embodiment, the complex is an antibody detecting complex, thus the biological molecule detected by the phage-particle complex is an antibody. In one embodiment, the antibody is an autoantibody. The probe is capable of being specifically bound by an antibody. In one embodiment, the probe is a polypeptide probe. In one embodiment, the polypeptide probe is present on a phage, wherein the phage is linked or coupled to the particle. The phage can be linked covalently or non-covalently to the particle. In one embodiment, a linker is incorporated between the phage and the particle. The linker can covalently or non-covalently couple the phage and particle. In another embodiment, a linker is incorporated between the phage and the probe. The linker can covalently or non-covalently couple the phage and probe.

In one embodiment, the phage-particle complex comprises a single probe, such as a single polypeptide probe. In another embodiment, the phage-particle comprises multiple probes. In another embodiment, the antibody detecting complex comprises a plurality of probes, such as at least 1, 2, 3, 4, 5, 6, 7, 8, 9, or 10 probes. In one embodiment, the phage-particle complex comprises multiple probes of the same type, such two or more polypeptide probes or two or more nucleic acid probes. In another embodiment, the phage-particle complex comprises multiple probes of different types, such as one or more polypeptide probes with one or more nucleic acid probes.

A plurality of phage-particle complexes can be used for high-throughput detection of molecules. While the phage provides a platform on which the probes can be presented to biological molecule for an opportunity of non-covalent binding, the particle provides a platform by which a user can store identification information corresponding to the nature of phage and the probe.

The encoding, i.e., assigning a particular code to a combination of a phage and probe and recording the code to a particular particle, can be achieved through controlled manufacturing process. For example, during the synthesis of phage-particle, a batch of phages having a known type of probes is prepared. A particle is given identification information, such as a fluorescent signal. The particular signal given to a particle is recorded. In the next step, the phage and the particle is coupled by methods disclosed herein. By doing so, a fluorescent signal, known to a user, encoded on the particle, is assigned to a phage with known type of probes.

The phage-particle complex can be used for detecting a condition. The phage portion of the complex can display one or more probes on the surface. The bead portion enables identification and quantitation of one or more molecules bound by the probe. A particle not only gives a unique identification to the probe it is coupled to, but also provides a means to be quantitated. For example, the collective intensity of fluorescent can be proportional to the amount of particles emitting fluorescent signals. Measuring the collective light intensity can thus provide a measurement for the quantity of antibodies or autoantibodies. Alternatively, the number of fluorescent particles can be counted by a machine. Such machine is equipped to pass the particle as a discrete unit, e.g., a single particle, through a light and the change in emission spectrum caused by the passing is recorded as an event corresponding to the passage of a single particle.

The target molecule, either by its identity or by its quantity, can provide an indication of a presence or absence of a disease or condition. For example, overexpression of a certain ligand can be an indication of a disease or condition. Presence of a certain gene product or antibody against can be an indication of a disease or condition. In one embodiment, detection of an antibody, such as an autoantibody is accomplished by contacting the antibody or autoantibody in a sample with a phage-particle complex. In one embodiment, detection comprises determining the presence or absence of one or more antibodies. In another embodiment, detection comprises quantitating the amount of antibodies in a sample.

Probe

The compositions and methods of the present disclosure relate to a complex comprising a probe, wherein the probe is displayed or present on a phage. A probe can be any biological molecule capable of binding to other biological target molecules. The probe can be, but not limited, to a protein, polypeptide, antibody, ligand, or nucleic acid. The probe can be DNA, RNA, a monoclonal antibody, polyclonal antibody, Fab, Fab′, single chain antibody, synthetic antibody, aptamer (DNA/RNA), peptoid, zDNA, peptide nucleic acid (PNA), locked nucleic acid (LNA), lectin, synthetic or naturally occurring chemical compound (including but not limited to a drug or labeling reagent), dendrimer, or a combination thereof. In one embodiment, the probe is a polypeptide. In another embodiment the probe is an antigen. In another embodiment, the probe is a receptor or a portion of a receptor. In another embodiment, the probe is a ligand. In another embodiment the probe is a nucleic acid. In another embodiment the probe is a bait molecule that interacts with a specific target molecule (such as a protein or nucleic acid molecule).

In one embodiment, the probe is capable of being specifically bound by an antibody. In a further embodiment, the antibody is an autoantibody. In one embodiment, the level, presence, or absence of an antibody in a sample can be determined by detecting the binding of one or more antibodies to a polypeptide probe. In one embodiment, an antibody is an autoantibody. An autoantibody refers to an antibody produced by a host (with or without immunization) and directed to a host antigen (such as a tumor antigen). Tumor-associated antigens recognized by humoral effectors of the immune system are an attractive target for diagnostic and therapeutic approaches to human cancer.

The binding of an antibody with a polypeptide probe can be specific, such that the interaction of the autoantibody with the polypeptide probe is dependent upon the presence of a particular structure (i.e., the antigenic determinant or epitope) of the polypeptide probe. Antigenic determinates or epitopes can comprise amino acids in linear or non-linear sequence in a polypeptide probe and can also comprise one or more amino acids which are in proximity to each other via protein folding (e.g., conformational epitopes). Thus, a single polypeptide or protein can potentially be bound by multiple antibodies or autoantibodies which recognize different epitopes. In some instances, known epitopes of a particular polypeptide can be used as a probe to detect for the presence, absence or level of antibodies or autoantibodies which bind a particular epitope.

The polypeptide probe can be an antigen identified through serologic identification of antigens, for example by recombinant expression cloning (SEREX), such as described by Kim et al., Biotech. Lett. (2004); 26: 585-588. Generally, in this method, an antigen can be identified by screening expression cDNA libraries from human solid tumors with sera of autologous patients. This type of screening of a cDNA expression library by conventional methods typically requires the preparation of a large number of membrane filters blotted with bacteriophage plaques that are then searched with a specific probe. In the case of the SEREX experiments, the screening is performed using sera from cancer patients, which can be in very limited quantities.

A polypeptide probe for detecting an antibody can also be identified by phage-display technology, which can be based on the insertion of foreign nucleotide sequences into genes encoding for various capsid proteins of T7 phage, resulting in a heterogeneous mixture of phages, each displaying the different peptide sequence encoded by a corresponding insert. A physical link between a displayed fusion protein and DNA encoded for it make this phage target selectable. The phage target can express or display a polypeptide probe, which can be used to detect antibodies that are produced by a subject, or autoantibodies, which can then be used to detect a cancer. The polypeptide probe can be displayed by a phage and used to detect an antibody from a sample obtained from a subject. In one embodiment, an antibody is an autoantibody.

Polypeptide is used in its broadest sense and may include a sequence of subunit amino acids, amino acid analogs, or peptidomimetics. The subunits may be coupled by peptide bonds. The polypeptides can be naturally occurring, processed forms of naturally occurring polypeptides (such as by enzymatic digestion), chemically synthesized or recombinantly expressed. The polypeptides for use in the methods of the present invention can be chemically synthesized using standard techniques. The polypeptides can comprise D-amino acids (which are resistant to L-amino acid-specific proteases), a combination of D- and L-amino acids, β amino acids, or various other designer or non-naturally occurring amino acids (e.g., β-methyl amino acids, Cα-methyl amino acids, and Nα-methyl amino acids, etc.) to convey special properties. Synthetic amino acids may include ornithine for lysine, and norleucine for leucine or isoleucine. In addition, the polypeptides can have peptidomimetic bonds, such as ester bonds, to prepare polypeptides with novel properties. For example, a polypeptide may be generated that incorporates a reduced peptide bond, i.e., R₁—CH₂—NH—R₂, where R₁ and R₂ are amino acid residues or sequences. A reduced peptide bond may be introduced as a dipeptide subunit. Such a polypeptide would be resistant to protease activity, and would possess an extended half-live in vivo. Polypeptides can also include peptoids (N-substituted glycines), in which the side chains are appended to nitrogen atoms along the molecule's backbone, rather than to the α-carbons, as in amino acids. Polypeptides and peptides are intended to be used interchangeably throughout this application, i.e. where the term peptide is used, it may also include polypeptides and where the term polypeptides is used, it may also include peptides.

The polypeptide probe can be a fragment or portion of a larger protein. The fragment can range in size from two amino acid residues to the entire amino acid sequence minus one amino acid. In one embodiment, a polypeptide probe is a fragment of an untranslated region (UTR) of a protein, such as a fragment that is encoded by a nucleic sequence that is a UTR region of a gene, such as the 5′ or 3′ UTR of a gene.

The fragment can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 amino acids in size. In one embodiment, the fragment is less than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 amino acids in size. A polypeptide probe useful in the compositions and methods herein, regardless of size, is capable of specific interaction with an antibody, such as an autoantibody.

In one embodiment, a polypeptide probe can be a fragment of a protein encoded by a gene, or a region upstream or downstream of a coding sequence, such as a UTR region, of a gene listed in Table 1. In one embodiment, a polypeptide probe is a fragment of a protein encoded by a gene, or a fragment encoded by a sequence of a UTR region of a gene. In one embodiment, the gene can be alpha-2 glycoprotein1 (AZGP1), DCHS1 (protocadherin-16 precursor), Homo sapiens family with sequence similarity 53, member B (FAM53B), mitogen-activated protein kinase kinase kinase 9 (MAPKKK9), Deaminase Domain, ADP-ribosylation factor 6 (ARF6), Centrosomal Protein 164 kDa (CEP 164), ADAM metallopeptidase domain 9 (ADAMS), splicing factor, arginine/serine-rich 14 (SFRS14), cytochrome c oxidase subunit Va (Cox5a), TIMP metallopeptidase inhibitor 2 (TIMP2), breast cancer metastasis-suppressor 1-like (BRMSL1), RP3-323M22 (Nucleolin), Homo sapiens chromosome 3 genomic contigs (NKX3-1), casein kinase 2, alpha prime polypeptide (CSNK2A2), Desmocollin 3 (DSC3), ribosomal protein L34 (RPL34), Chromosome 3 UTR region ropporin/RhoEGF (3′ UTR Ropporin), aurora kinase A interacting protein 1 (AURKAIP1), 5′-UTR BMI1, ribosomal protein SA (LAMR1), Contig GRCh37.p2 (1371451::1372008), Prostate Specific Antigen (PSA), ribosomal protein SA (RPSA) (minus strand), RNA Binding Protein 6 (RBM6) (minus strand), or hypothetical protein XP_{—}353238. Other suitable markers can include those known in the art, such as biomarkers disclosed in U.S. patent application Ser. No. 13/050,544, U.S. patent application Ser. No. 13/072,542, and U.S. Pat. No. 7,858,323, which are hereby incorporated by reference in their entirety. A polypeptide probe can comprise a peptide sequence, or fragment thereof, such as those listed in Table 1. In one embodiment, a polypeptide probe comprises SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 74, SEQ ID NO: 77, SEQ ID NO: 80, or SEQ ID NO: 82, or a fragment thereof. In another embodiment, a polypeptide probe comprises a polypeptide encoded by SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 75, SEQ ID NO: 78, SEQ ID NO: 81, or SEQ ID NO: 83, or a fragment thereof. In another embodiment, a polypeptide probe comprises a polypeptide encoded by SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65 SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 76, or SEQ ID NO: 79, or a fragment thereof

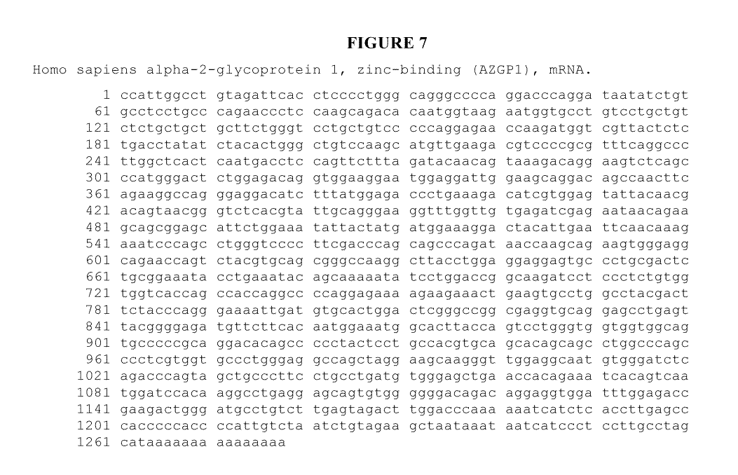

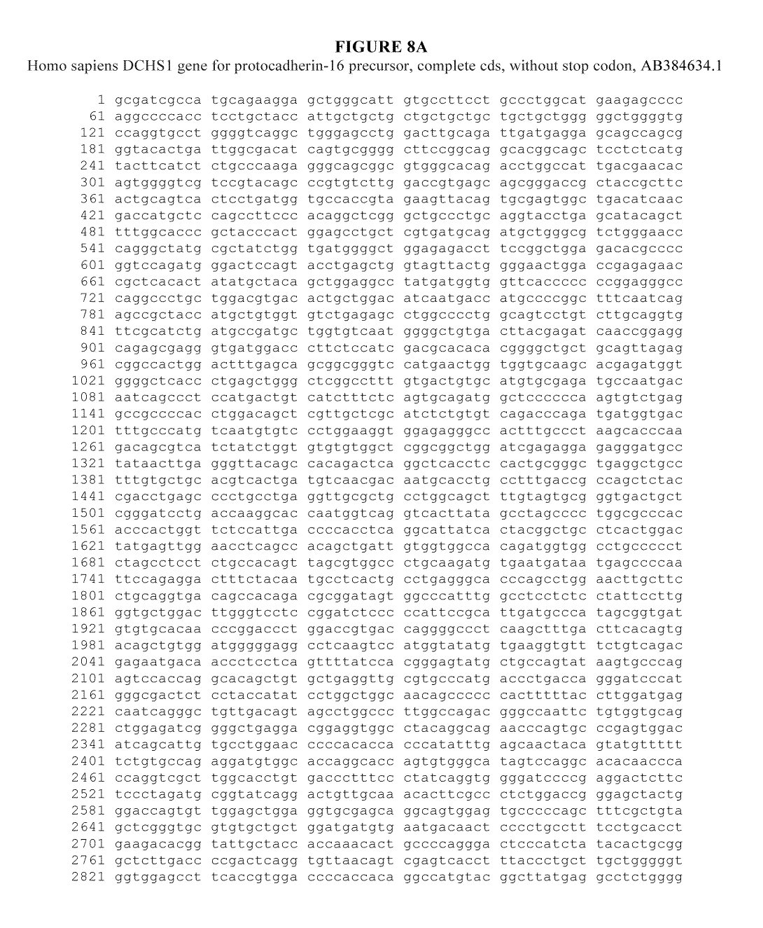

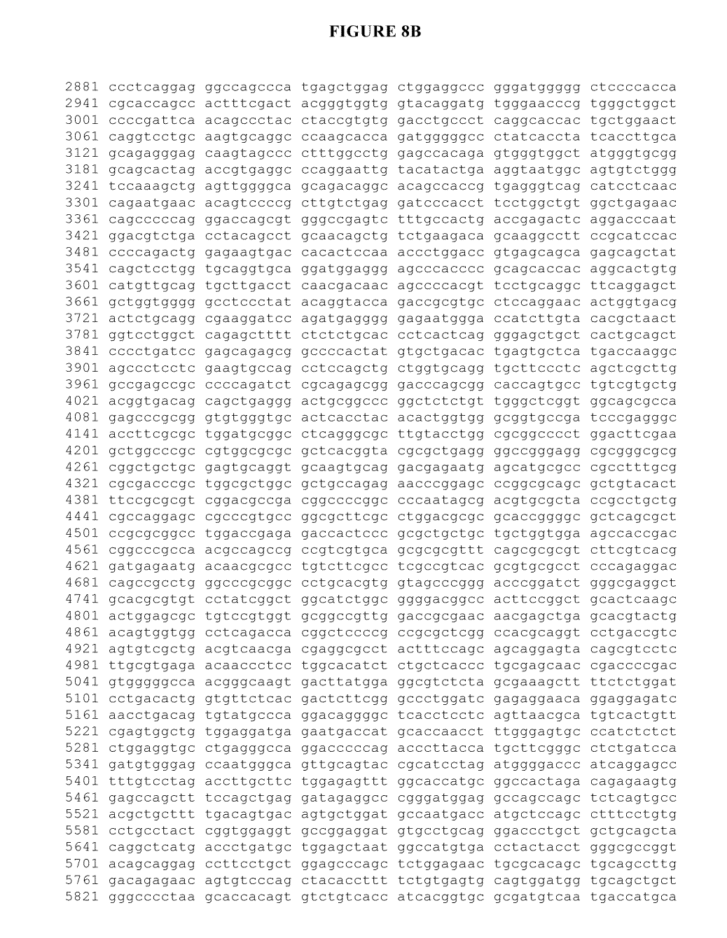

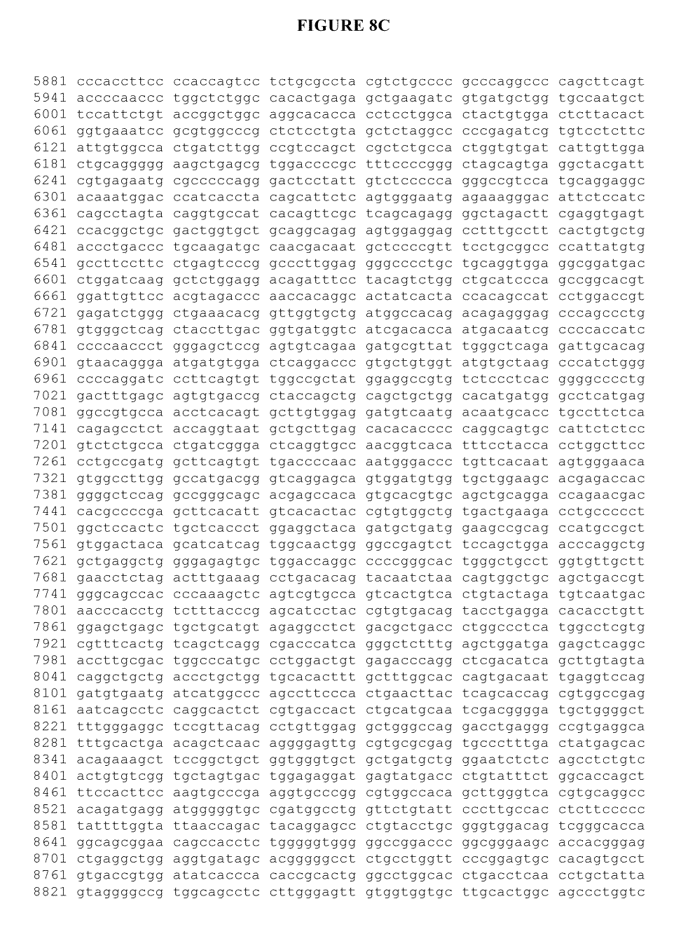

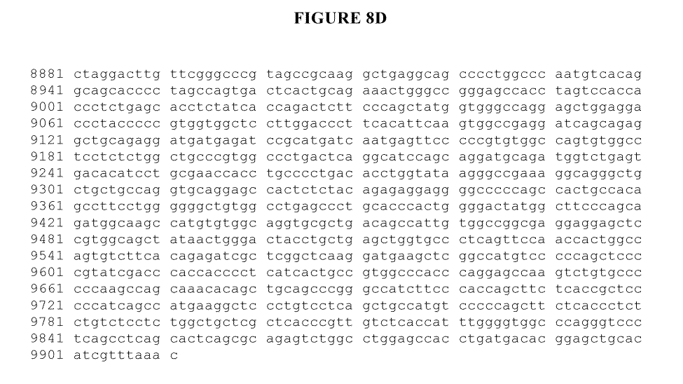

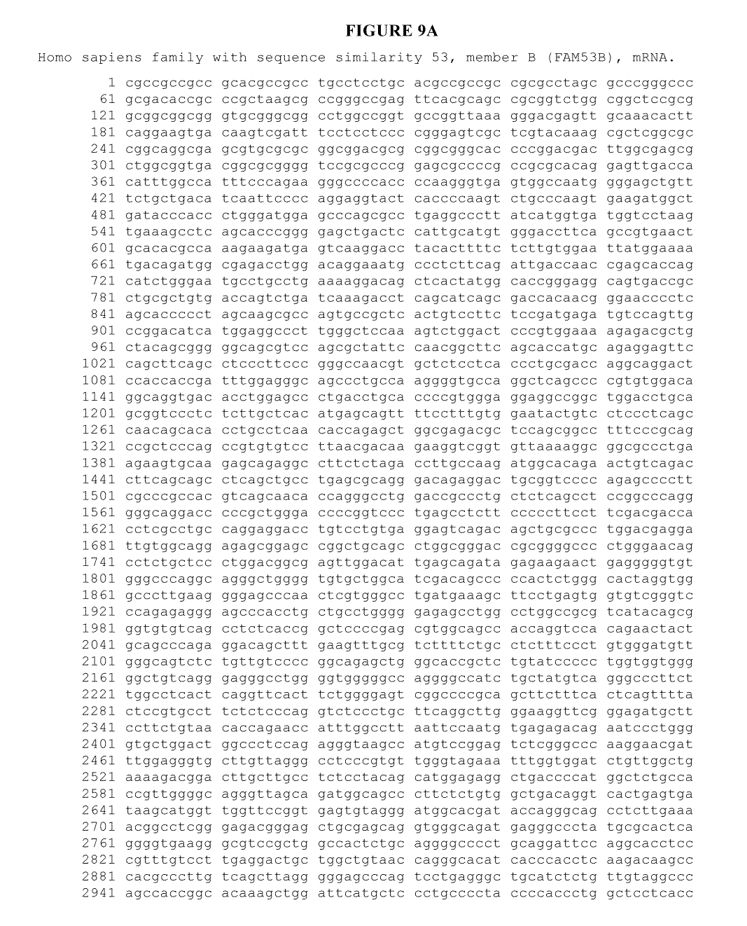

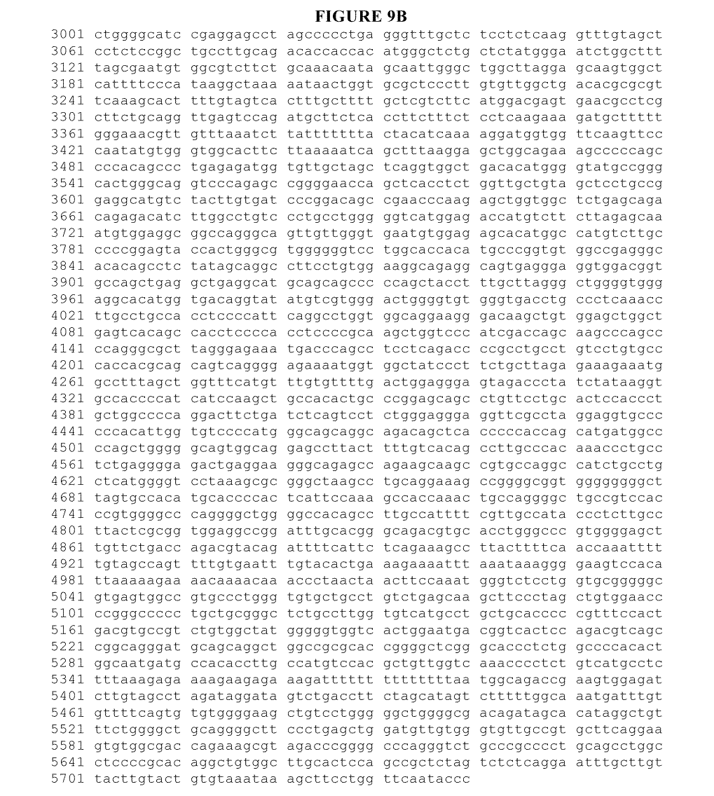

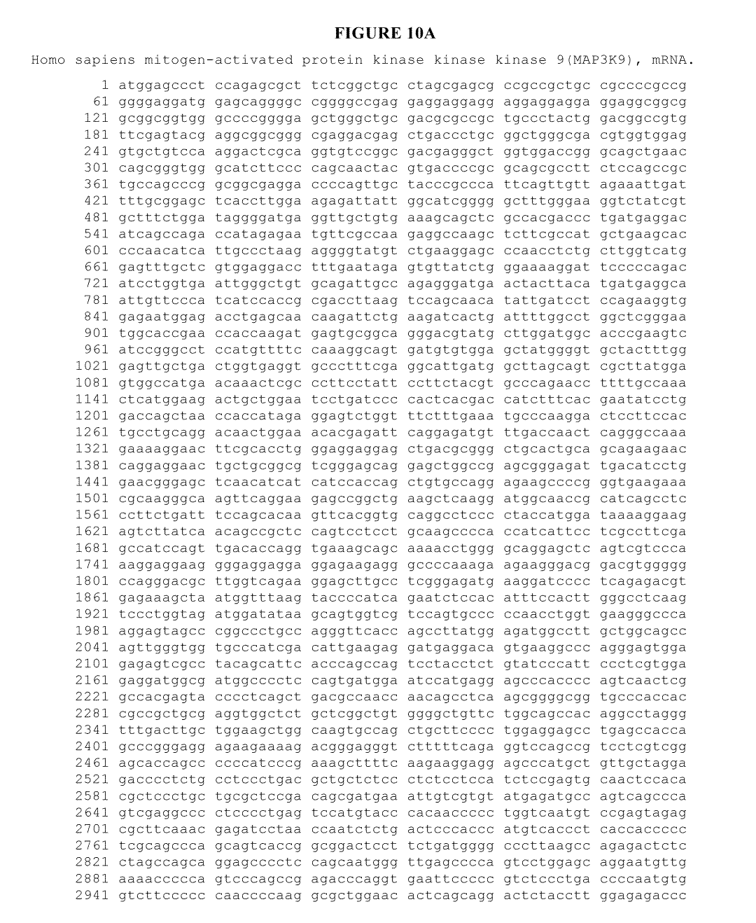

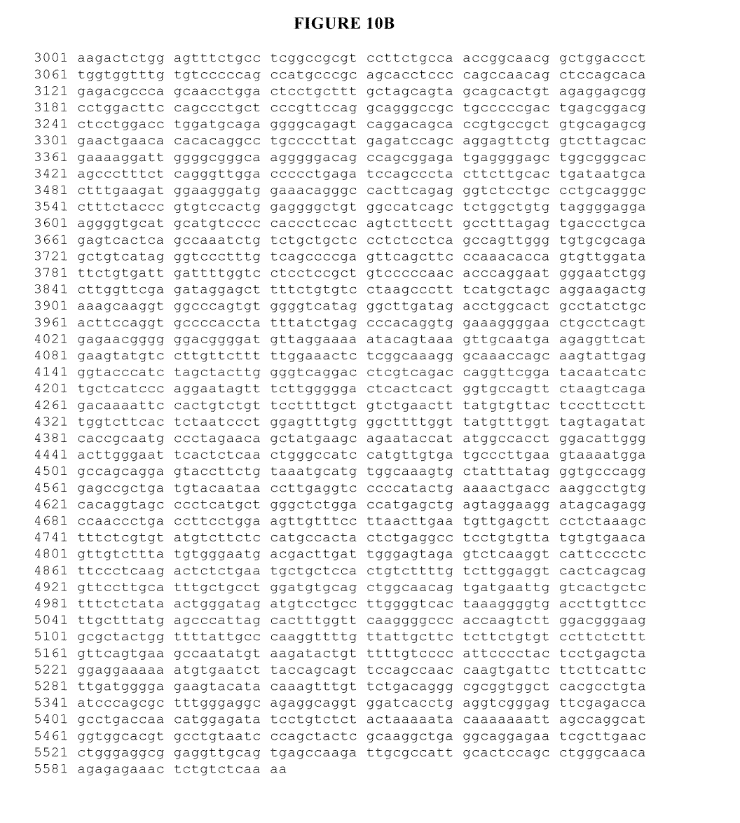

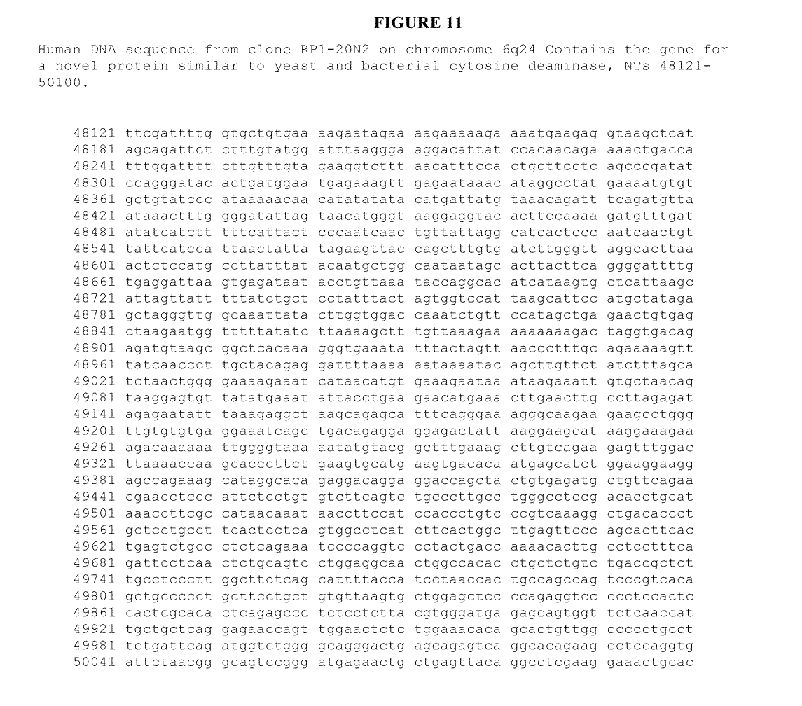

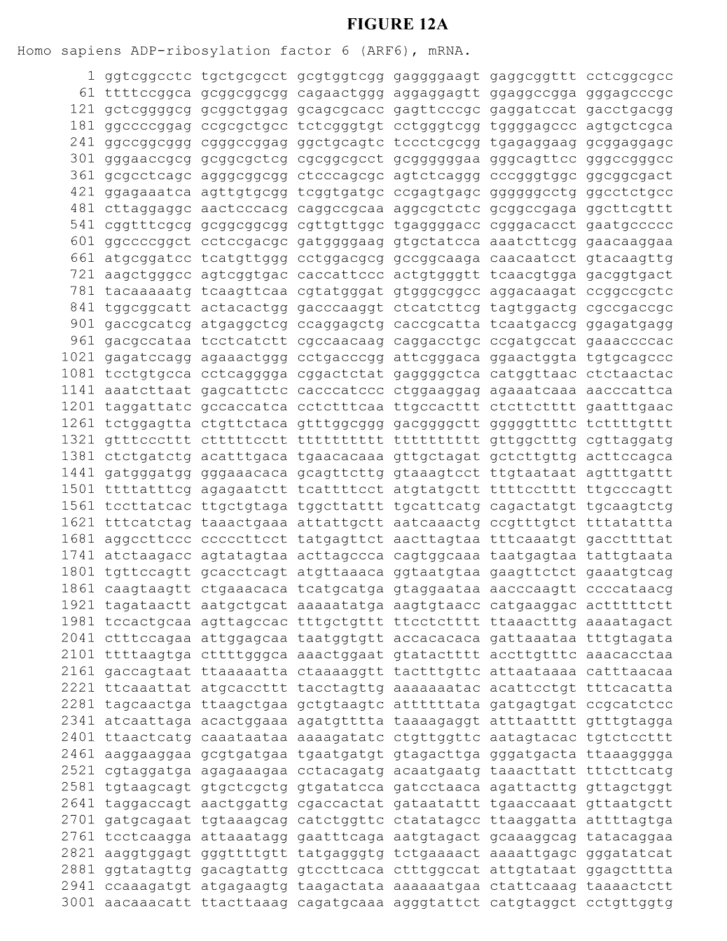

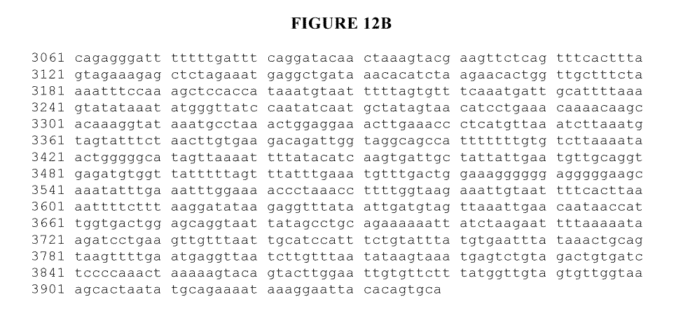

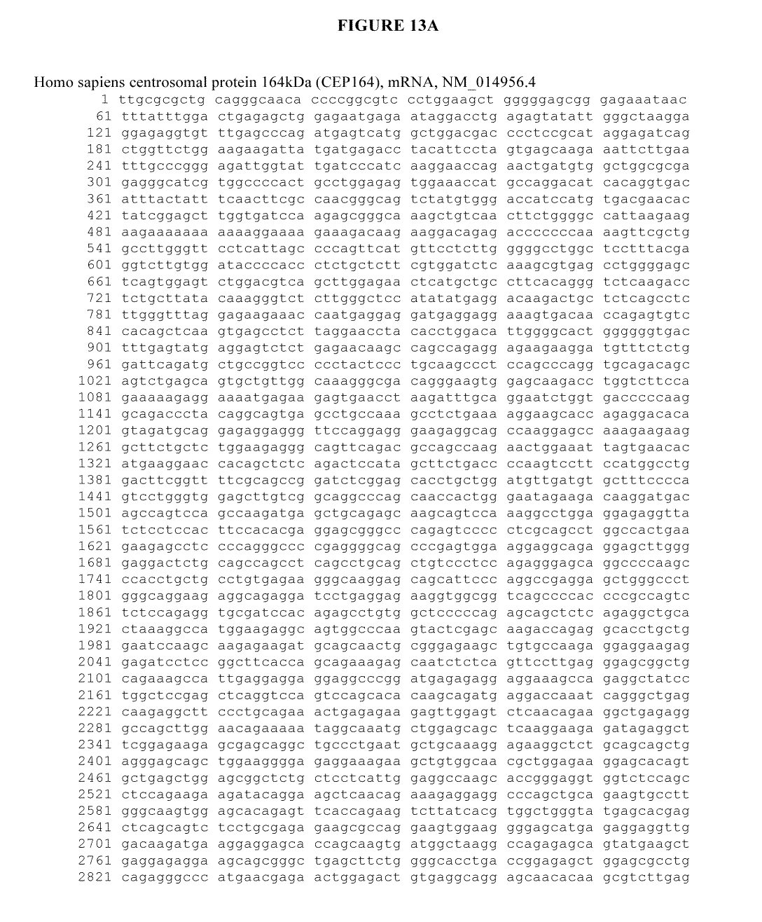

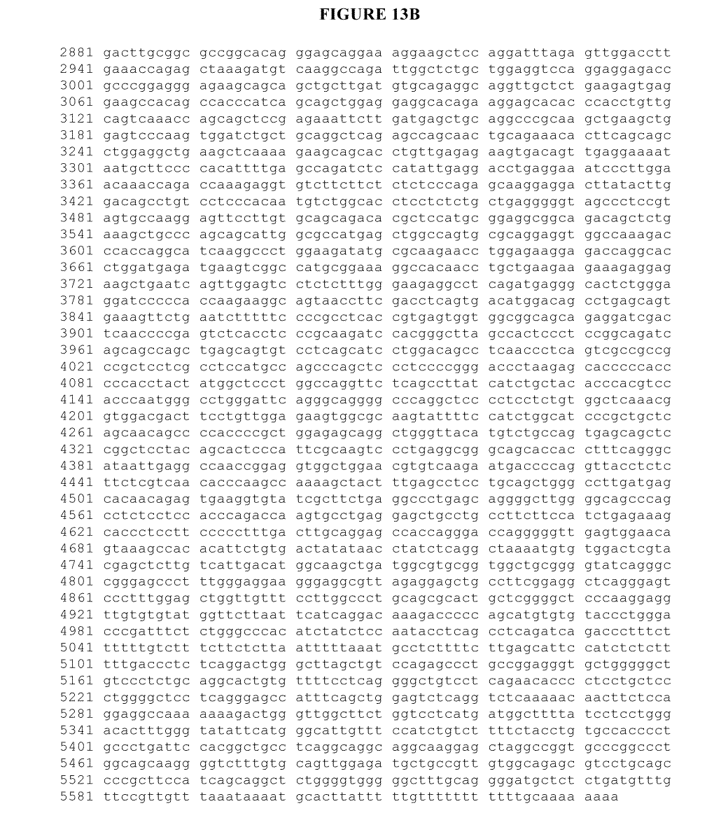

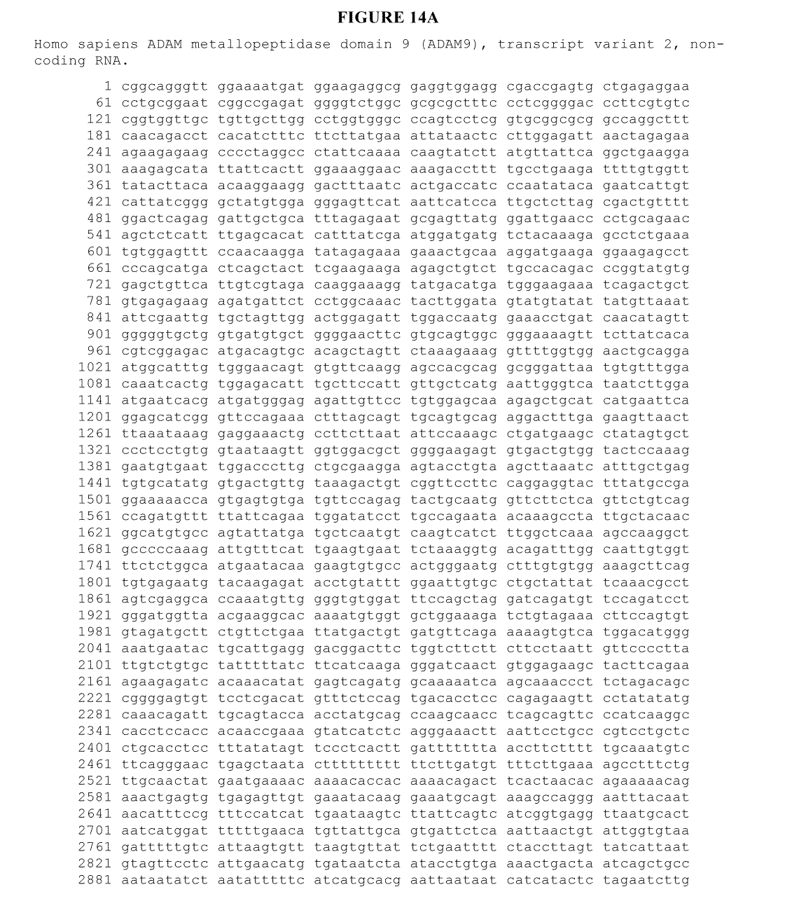

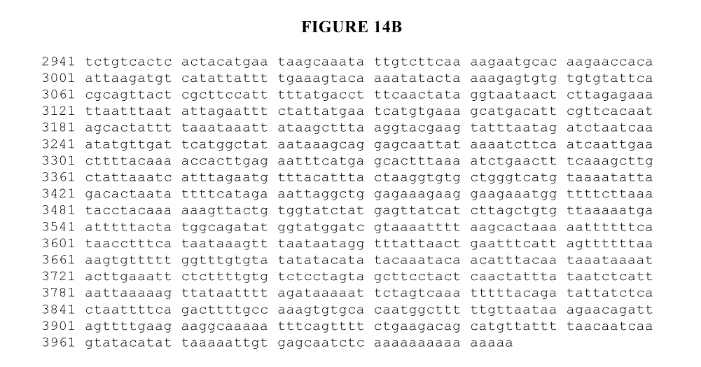

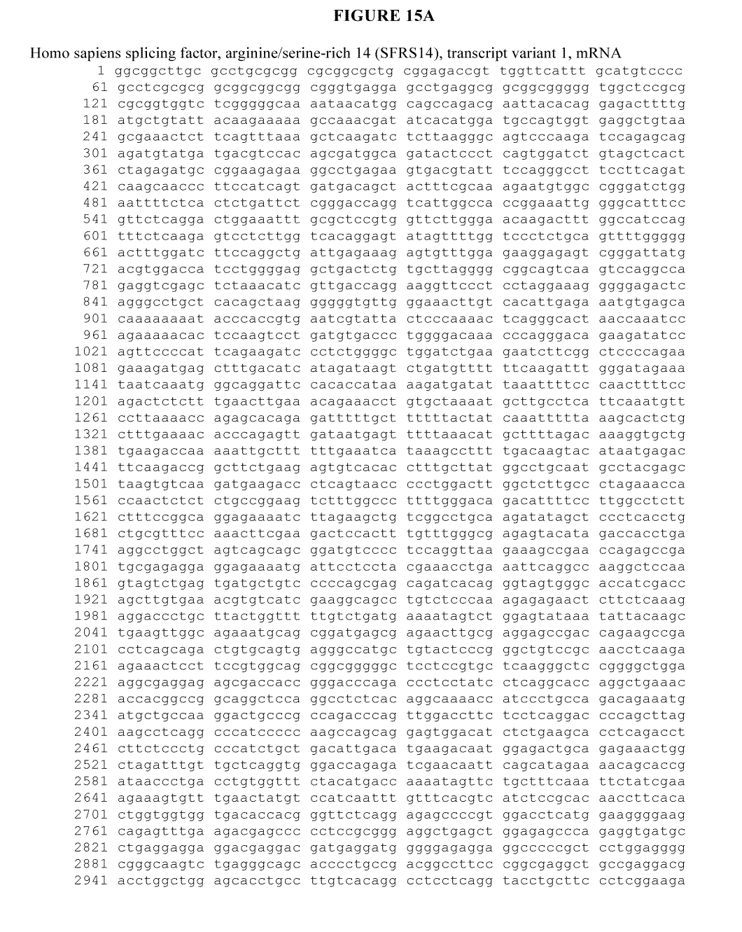

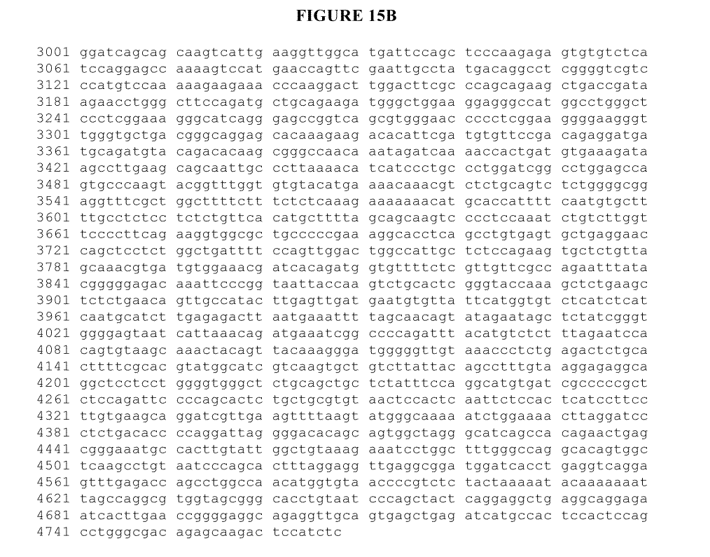

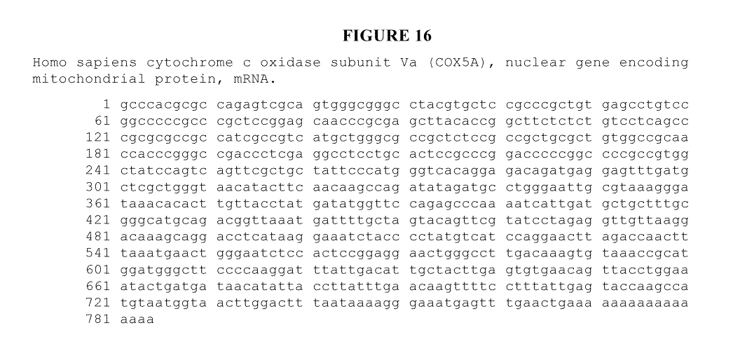

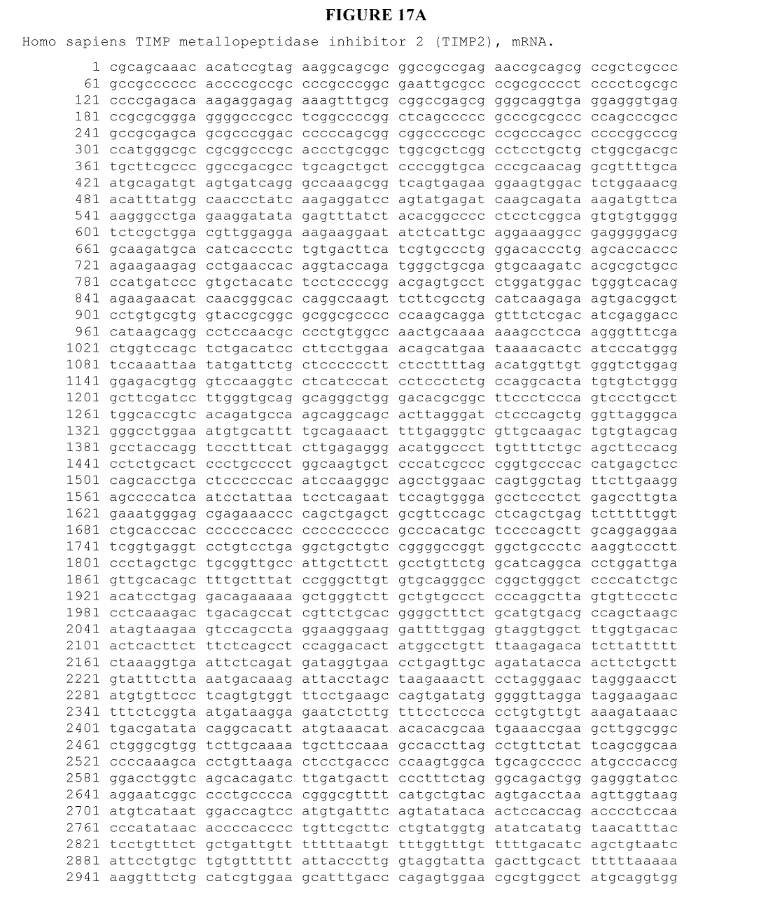

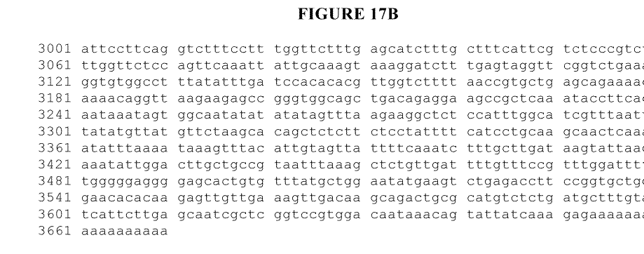

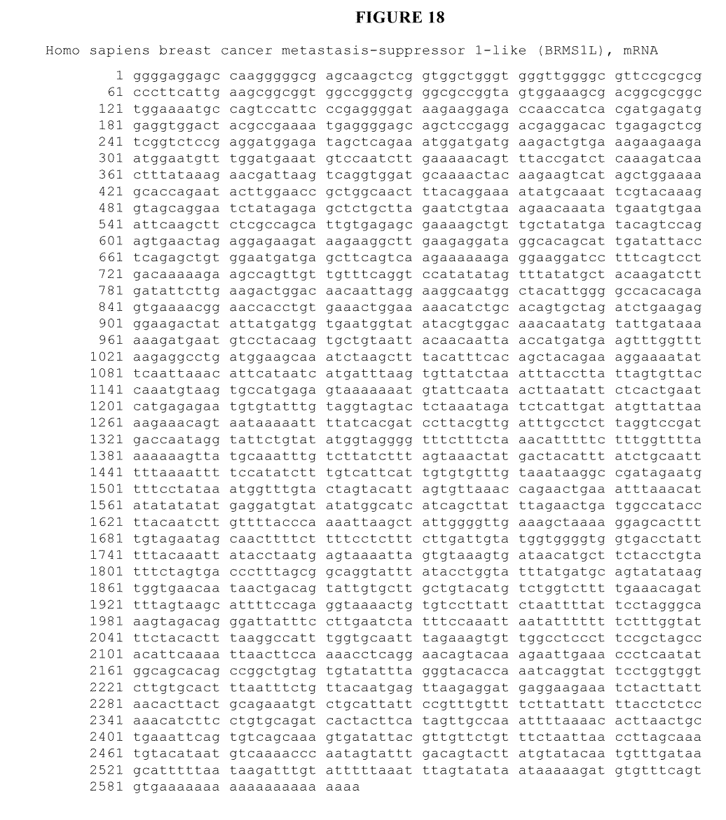

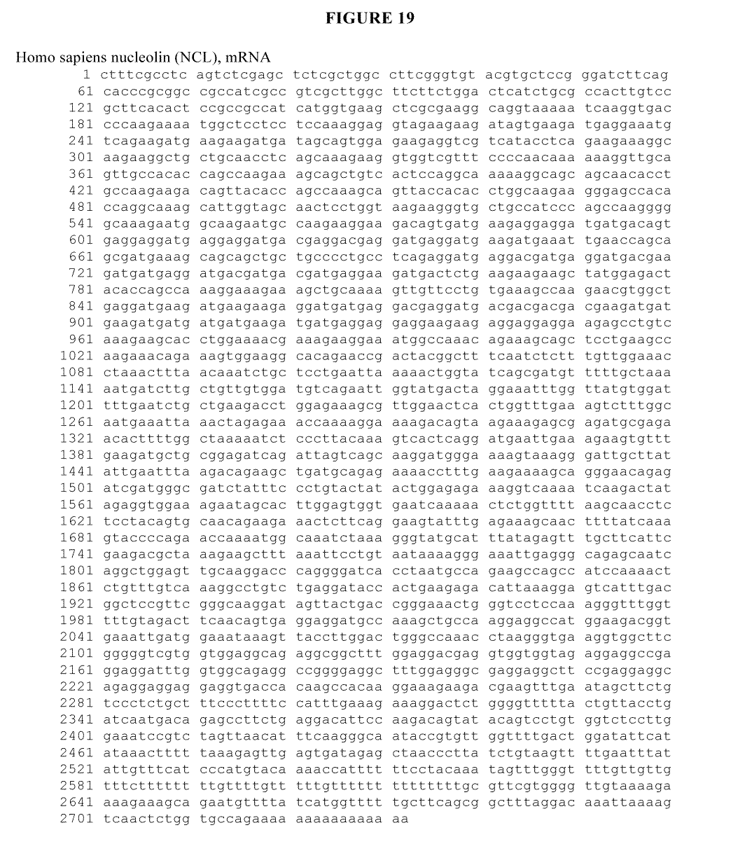

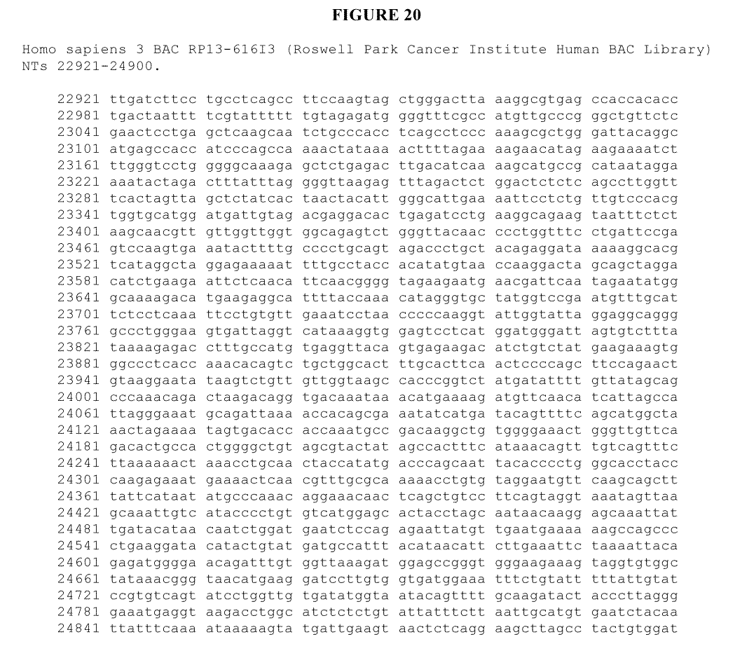

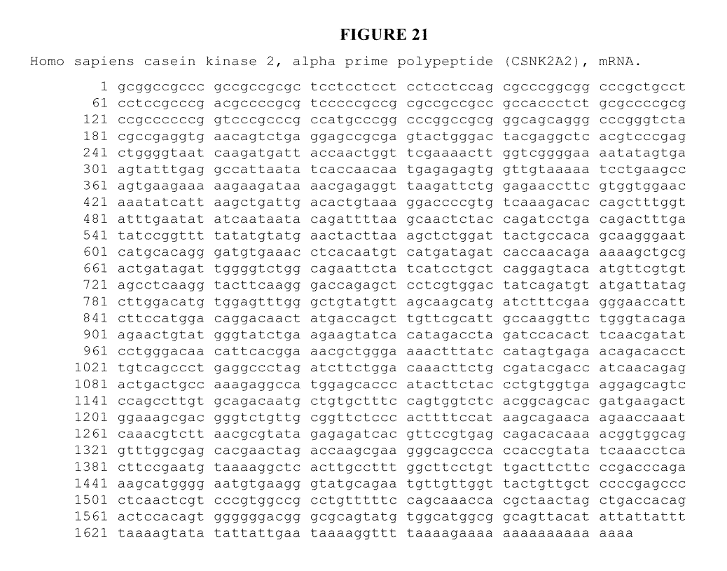

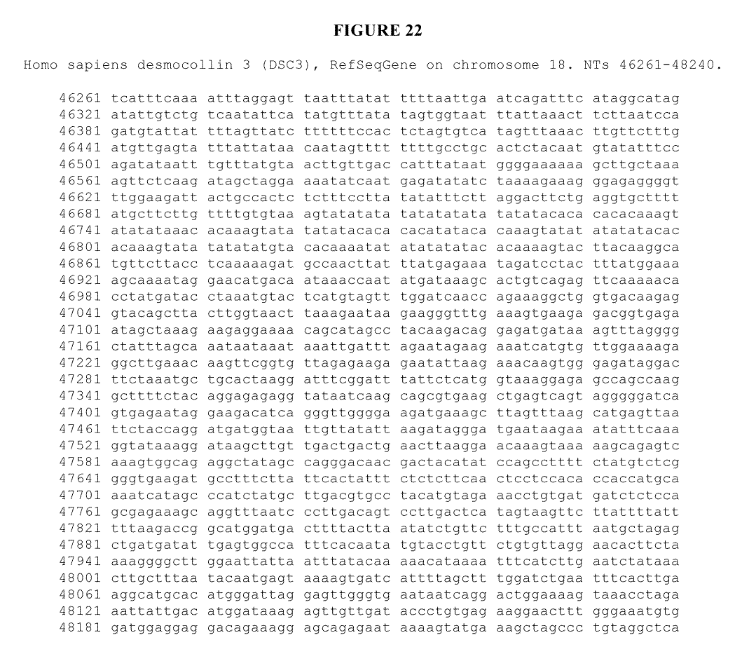

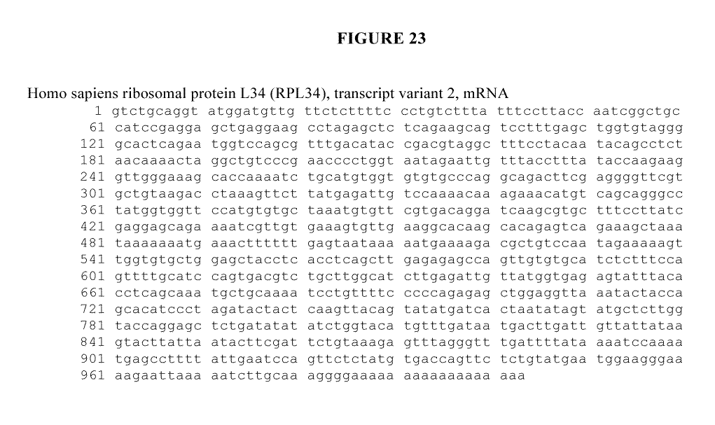

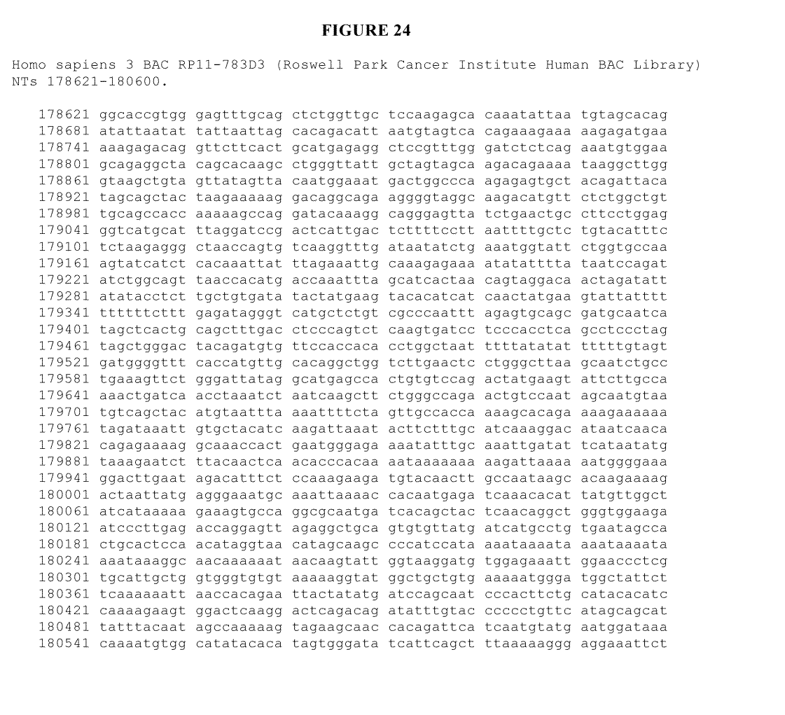

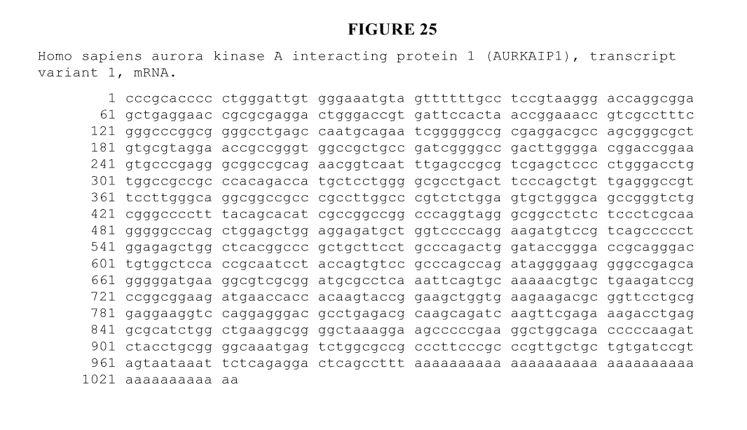

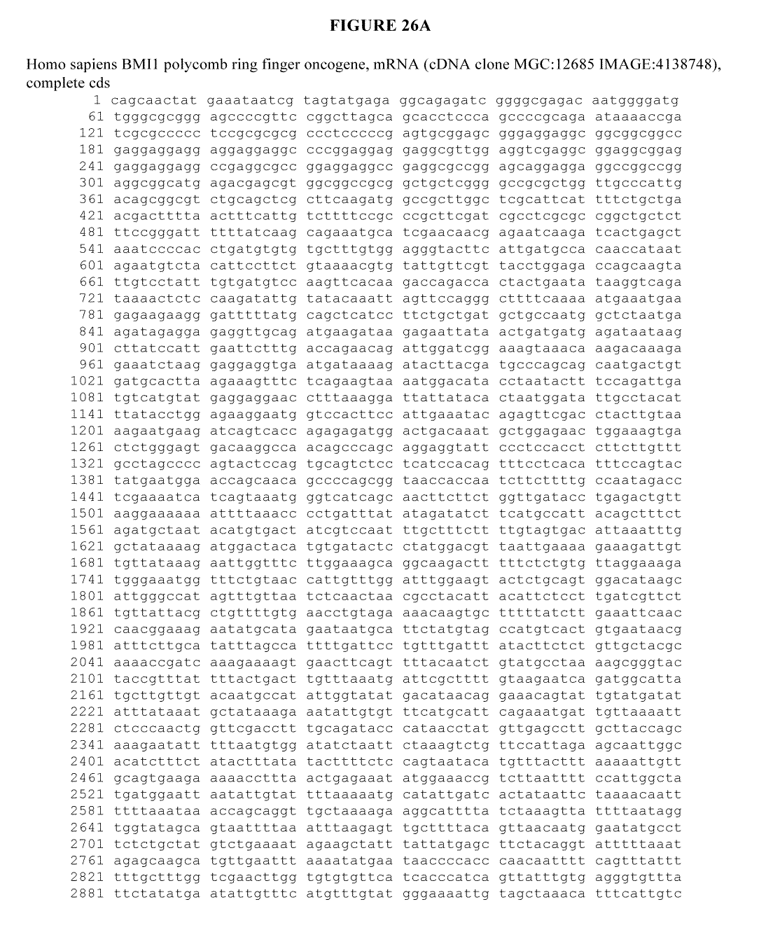

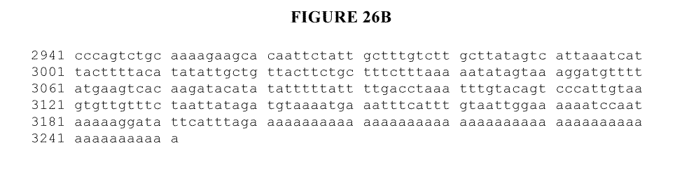

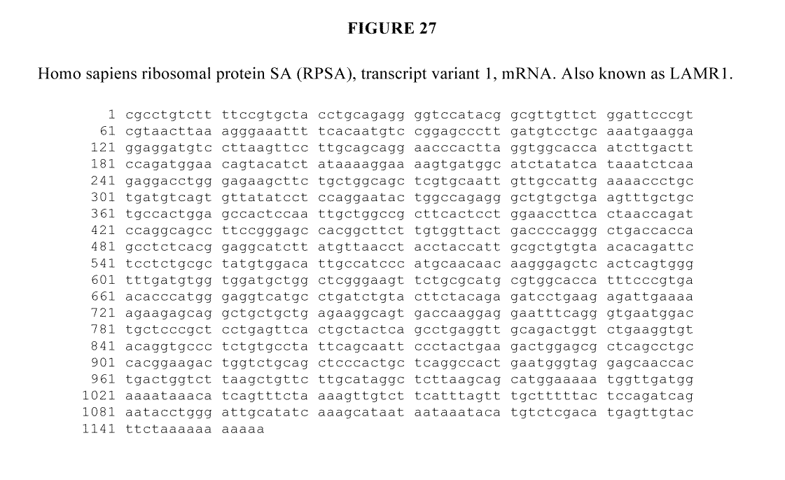

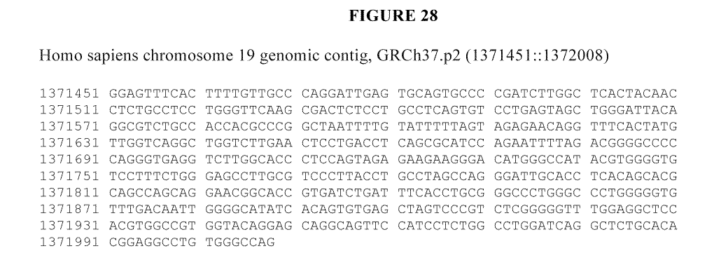

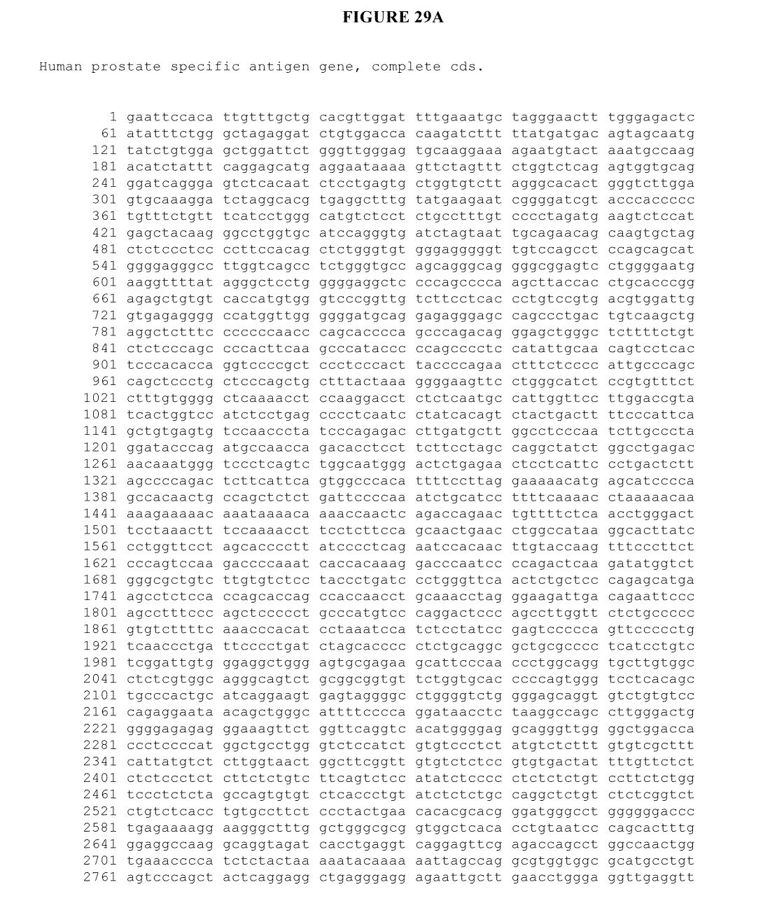

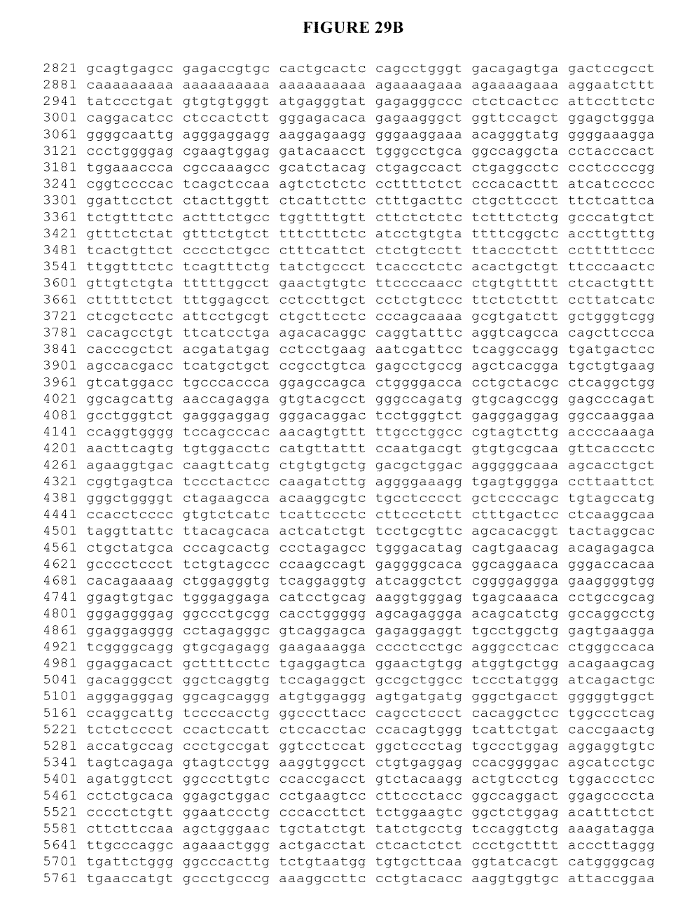

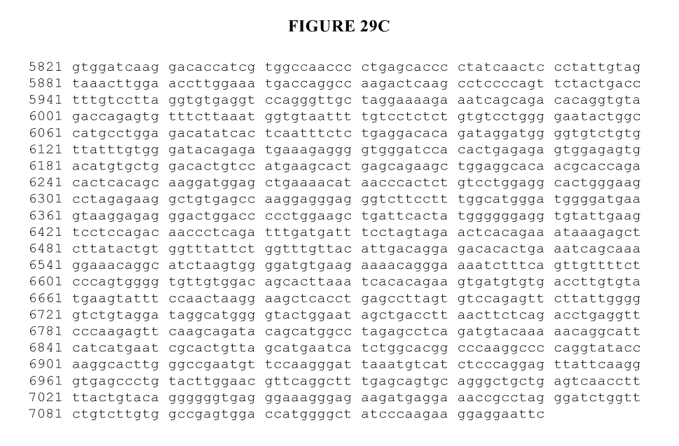

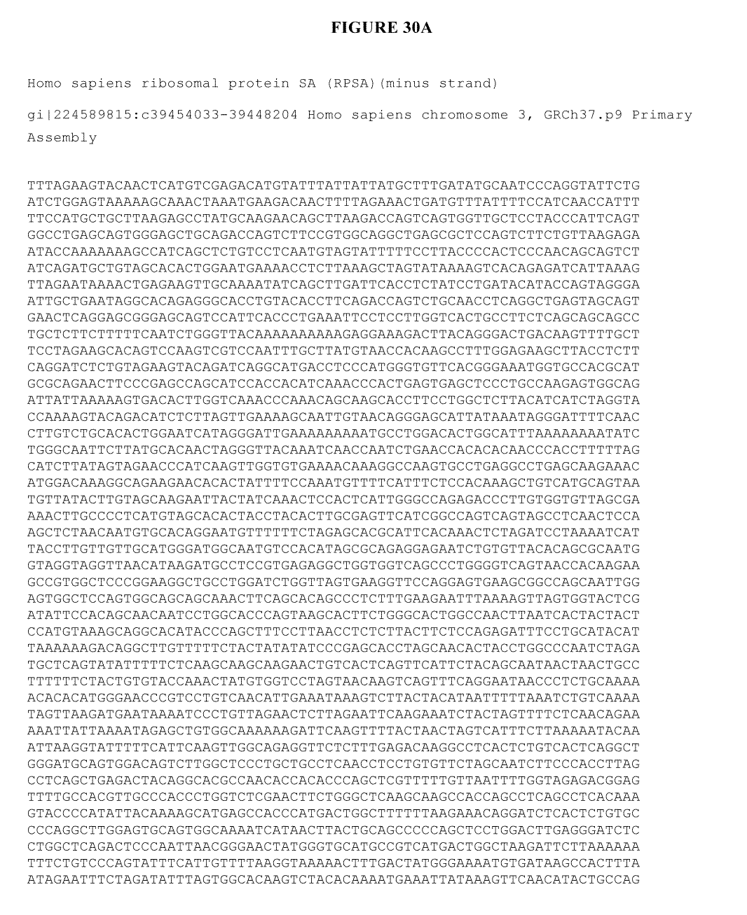

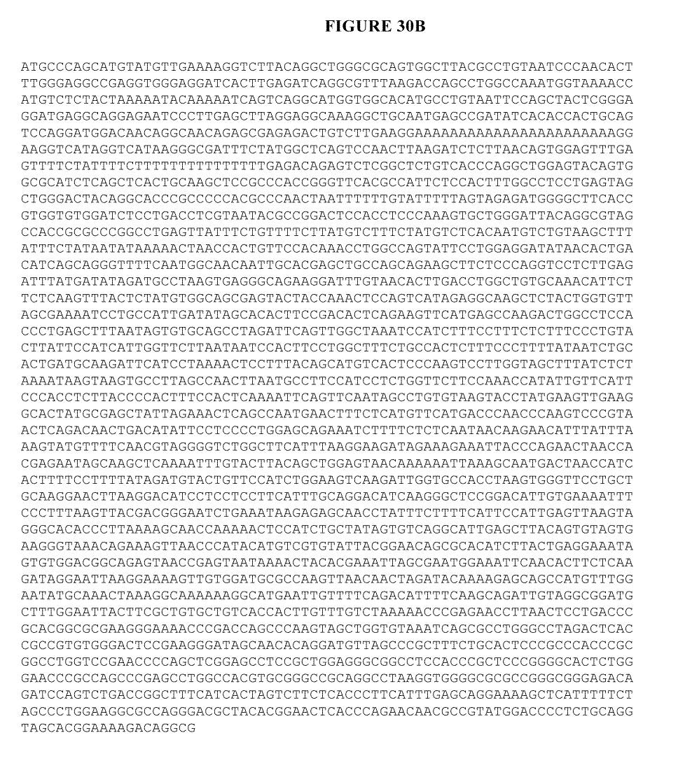

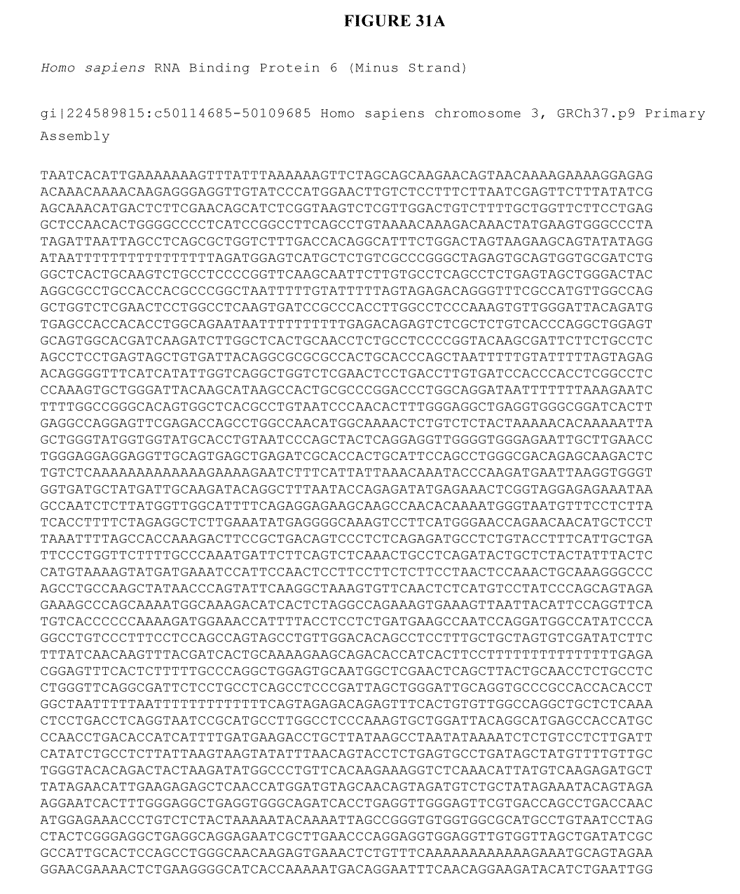

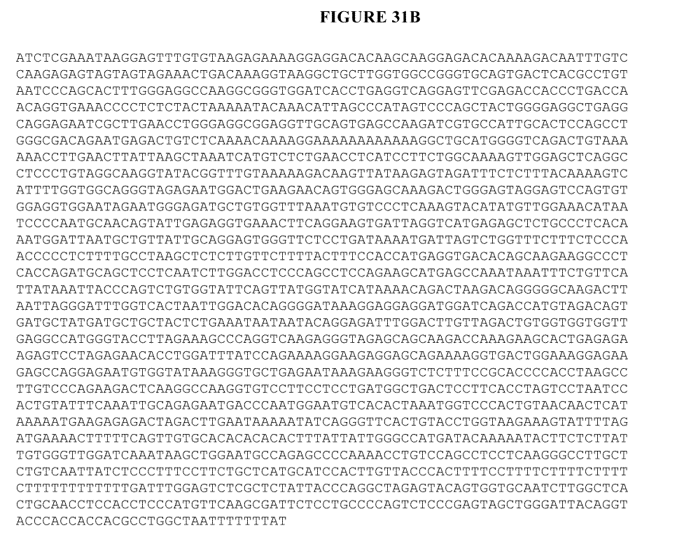

TABLE 1

| | | | | | Clone DNA Sequence |
|---|---|---|---|---|---|
| Clone | | NCBI | Gene | Peptide | (Encoding Peptide |
| ID | Gene | Designation | Sequence | Sequence | Sequence) |
| 3F5 | alpha-2 | NM_001185.3 | FIG. 7 | SSVPPQDTAP | TCAAGCGTGCCCCCGCAGGA |
| | glyco | | (SEQ ID | YSCHVQHSSL | CACAGCCCCCTACTCCTGCC |
| | protein1 | | NO: 51) | AQPLVVPWE | ACGTGCAGCACAGCAGCCTG |
| | (AZGP1) | | | AS | GCCCAGCCCCTCGTGGTGCC |
| | | | | (SEQ ID NO: 1) | CTGGGAGGCCAGCTAGGAAG |
| | | | | | CAAGGGTTGGAGGCAATGTG |
| | | | | | GGATCTCAGACCCAGTAGCT |
| | | | | | GCCCTTCCTGCCTGATGTGG |
| | | | | | GAGCTGAACCACAGAAATCA |
| | | | | | CAGTCAATGGATCCACAAGG |
| | | | | | CCTGAGGAGCAGTGTGGGGG |
| | | | | | GACAGACAGGAGGTGGATTT |
| | | | | | GGAGACCGAAGACTGGGATG |
| | | | | | CCTGTCTTGAGTAGACTTGG |
| | | | | | ACCCAAAAAATCATCTCACC |
| | | | | | TTGAGCCCACCCCCACCCCA |
| | | | | | TTGTCTAATCTGTAGAAGCC |
| | | | | | GGAAGCTTGCGGCCGCACTC |
| | | | | | GAGTAACTAGTTAACCCCTT |
| | | | | | GGGGCCTCTAAACGGGTCTT |
| | | | | | GAGGGGTTANCTNGTTNCTC |
| | | | | | GNGTGCGGCCGCNNGCTTCC |
| | | | | | GGCTTCTNCNGNTTNGNCNN |
| | | | | | TG N |
| | | | | | (SEQ ID NO: 26) |
| 2E11 | DCHS | AB384634.1 | FIG. 8 | PQTTAPRRAR | AGCTTTCGCTAGAGACGCCT |
| | 1 | | (SEQ ID | PRRS (SEQ ID | CCATAAGTCACTTGCCCGTT |
| | (protocadherin- | | NO: 52) | NO: 2) | GGCCCCCACGATCGGGGTCG |
| | 16 precursor) | | | | GTTGCTCGCAGGGCTGAGCA |
| | | | | | GAGATGTGCCAGGAGGGTTG |
| | | | | | TTCTCACGCAAGAGGACGCT |
| | | | | | GTACTCCTGCTGCTGGAAAG |
| | | | | | TAGGCGCCTCGTCGTTGACG |
| | | | | | TCAGCGACACTGACGGTCAG |
| | | | | | GACCTGCGTGGCCGAGCGCG |
| | | | | | GCGGGGAGCCGTGGTCTGAG |
| | | | | | G (SEQ ID NO: 27) |
| 17B8 | FAM5 | NM_014661.3 | FIG. 9 | EVHIKKKTKQ | GGGAAGTCCACATTAAAAAG |
| | 3B | | (SEQ ID | TLTNFQMGLL | AAAACAAAACAAACCCTAAC |
| | | | NO: 53) | VRGREWPCP | TAACTTCCAAATGGGTCTCCT |
| | | | | GCAACLSKLP | GGTGCGGGGGCGTGAGTGGC |
| | | | | (SEQ ID NO: 3) | CGTGCCCTGGGTGTGCTGCC |
| | | | | | TGTCTGAGCAAGCTTCCCTA |
| | | | | | GCTGTGGAACCCCGGGCCCC |
| | | | | | CTGCTGCGGGCTCTGCCTTG |
| | | | | | GTGTCATGCCTGCTGCACCC |
| | | | | | CCGTTTCCACTGACGTGCCGT |
| | | | | | CTGTGGCTATGGGGGTGGTC |
| | | | | | ACTGGAATGACGGTCACTCC |
| | | | | | AGACGTCAGCCGGCAGGGAT |
| | | | | | GCAGCAGGCTGGCCGCGCA |
| | | | | | (SEQ ID NO: 28) |
| 3F6 | MAPK | NM_033141.2 | FIG. 10 | SSGSGESRLQ | TCAAGCGGGAGTGGAGAGA |
| | KK9 | | (SEQ ID | HSPSQSYLCIP | GTCGCCTACAGCATTCACCC |
| | | | NO: 54) | FPRGEDGDGP | AGCCAGTCCTACCTCTGTATC |
| | | | | SSDGIHEEPTP | CCATTCCCTCGTGGAGAGGA |
| | | | | VNSATSTPQL | TGGCGATGGCCCCTCCAGTG |
| | | | | TPTNSLKRGG | ATGGAATCCATGAGGAGCCC |
| | | | | AHHRRCEVA | ACCCCAGTCAACTCGGCCAC |
| | | | | LLGCGAVLA | GAGTACCCCTCAGCTGACGC |
| | | | | ATGLGFDLLE | CAACCAACAGCCTCAAGCGG |
| | | | | AGKCQLLPLE | GGCGGTGCCCACCACCGCCG |
| | | | | EPEPPAREEK | CTGCGAGGTGGCTCTGCTCG |
| | | | | KRREGLFQRS | GCTGTGGGGCTGTTCTGGCA |
| | | | | SRPRRSTSPPS | GCCACAGGCCTAGGGTTTGA |
| | | | | RKLFKKEEHQ | CTTGCTGGAAGCTGGCAAGT |
| | | | | ACGRTRVTS | GCCAGCTGCTTCCCCTGGAG |
| | | | | (SEQ ID NO: 4) | GAGCCTGAGCCACCAGCCCG |
| | | | | | GGAGGAGAAGAAAAGACGG |
| | | | | | GAGGGTCTTTTTCAGAGGTC |
| | | | | | CAGCCGTCCTCGTCGGAGCA |
| | | | | | CCAGCCCCCCATCCCGAAAG |
| | | | | | CTTTTCAAGAAGGAGGAGCA |
| | | | | | CCAAGCTTGCGGCCGCACTC |
| | | | | | GAGTAACTAGTTAACCCCTT |
| | | | | | GGGGCCTCTAAACGGGTCTT |
| | | | | | GAGGGGGTTANCTNGTTACT |
| | | | | | CGNGTGCGGCCGCNNGCTTG |
| | | | | | GTGCTCNNCNTTN (SEQ ID |
| | | | | | NO: 29) |
| 5F4 | Deaminase | AL031320.1 | FIG. 11 | VSGSQRVKYL | GAGATGTAAGCGGCTCACAA |
| | Domain | | (SEQ ID | LVNPLQKKFI | AGGGTGAAATATTTACTAGT |
| | Cont | | NO: 55) | NPCYRGF | TAACCCCCTTGCAGAAAAAG |
| | 1 | | | (SEQ ID NO: 5) | TTATCAACCCTTGCTACAGA |
| | | | | | GGATTTTAAAAAATAAAATA |
| | | | | | CAGCTTGTTCTATCTTTAGCA |
| | | | | | TCTAACTGGGGAAAAGAATC |
| | | | | | ATAACATGTGAAAGAATAAA |
| | | | | | TAAGAAATTGTGCTAACAGT |
| | | | | | AAGGAGTGTTATATGAAATA |
| | | | | | TTACCTGAAGAACATGAAAC |
| | | | | | TTGAACTTGCTAGAGATAGA |
| | | | | | GAATATTTAAAGAGGCTAAG |
| | | | | | CAGAGCATTTCAGGGAAAGG |
| | | | | | GCAAGAAGAAGCCTGGGTTG |
| | | | | | TGTGTGAGGAAATCAGCTGA |
| | | | | | CAGAGGAGGAGACTATTAAG |
| | | | | | GAAGCATAAGGAAAGAAAG |
| | | | | | ACAAAAAATTGGGGTAAAAA |
| | | | | | TATGTACGGCTTTGAAAGCT |
| | | | | | T (SEQ ID NO: 30) |
| 3C3 | ARF6 | NM_001663.3 | FIG. 12 | PKCRLQRQYT | GAAATGTAGACTGCAAAGGC |
| | | | (SEQ ID | GKGGVGFVY | AGTATACAGGAAAAGGTGGA |
| | | | NO: 56) | EGV | GTGGGTTTTGTTTATGAGGGT |
| | | | | (SEQ ID NO: 6) | GTCTGAAAACTAAAATTGAG |
| | | | | | CGGGATATCATGGTATAGTT |
| | | | | | GGACAGTATTGGTCCTTCAC |
| | | | | | ACTTTGGCCATATTGTATAAT |
| | | | | | GGAGCTTTTACCAAAGATGT |
| | | | | | ATGAGAAGTGTAAGACTATA |
| | | | | | AAAAAATGAACTATTCAAAG |
| | | | | | TAAAACTCTTAACAAACATT |
| | | | | | TTACTTAAAGCAGATGCAAA |
| | | | | | AGGGTATTCTCATGTAGGCT |
| | | | | | CCTGTTGGTGCAGAGGGATT |
| | | | | | TTTTTGATTTCAGGATACAAC |
| | | | | | TAAAGTACGAAGTTCTCAGT |
| | | | | | TTCACTTTAGTAGAAAGAGC |
| | | | | | TCTAGAAATGAGGCTGATAA |
| | | | | | ACACATCTAAGAACACTGGT |
| | | | | | TGCTTTCTAAAATTTCCAAAG |
| | | | | | CTCCACCATAAATGTAATTTT |
| | | | | | TAGTGTTTCAAATGATTGCAT |
| | | | | | TTTAAAGTATATAAATATGG |
| | | | | | GTTATCCAATATCAATGCTAT |
| | | | | | AGTAACATCCTGAAACAAAA |
| | | | | | CAAGCACAAAGGTATAAATG |
| | | | | | CCTAAACTGGAGGAAGCTTG |
| | | | | | (SEQ ID NO: 31) |
| 1B4A | Centrosomal | NM_014956.4 | FIG. 13 | PVSSSGSYSTP | TGGAGGAGAGGCTGGGCT GC |
| | Protein | | (SEQ ID | IRKSLRRAAPP | CCCAAGCCCCTGCTCAGGGC |
| | (CEP164) | | NO: 57) | FRA (SEQ ID | CTCAGAAGCCATACACCTTC |
| | (Minus strand) | | | NO: 7) | ACTCTGATTGTGCTCATCAA |
| | | | | | GGCCCAGCATGCAGGAGGCT |
| | | | | | CAAAGTAGCTTTTGGCTTGG |
| | | | | | GTGTTGACGAGAAGAGAGGT |
| | | | | | AACCTGGGGTCATTCTTGAC |
| | | | | | ACGTTCCAGCCACCTCCGGT |
| | | | | | TGGCCTCAATTATGCCCTGA |
| | | | | | AAGGTGGTGCTGCCCGCCTC |
| | | | | | AGGGACTTGCGAATGGGAGT |
| | | | | | GCTGTAGGAGCCGGAGCTGC |
| | | | | | TCACTGG (SEQ ID NO: 32) |
| 1H3 | ADAM | NR_027878.1 | FIG. 14 | NSGASGSRNF | TCGGCATAAAGTACCTCCTG |
| | metallo | | (SEQ ID | SSCSAEDFEK | GAAGGAACCGACAGTCTTTA |
| | peptidase | | NO: 58) | (SEQ ID NO: 8) | CAACAGTCACCATATGCACA |
| | domain | | | | CTCAGCAAATGATTTAAGCT |
| | 9 | | | | TACAGGTACTTCCTTCGCAG |
| | | | | | CAAGGGTCCAATTCACATTC |
| | | | | | CTTTGGAGTACCACAGTCAC |
| | | | | | ACTCTTCCCCAGCGTCCACC |
| | | | | | AACTTATTACCACAGGAGGG |
| | | | | | AGCACTATAGGCTTCATCAG |
| | | | | | GCTTTGGAATATTAAGAAGG |
| | | | | | CAGTTTCCTCCTTTATTTAAA |
| | | | | | GTTACTTCTCAAAGTCCTCTG |
| | | | | | CACTGCAACTGCTAAAGTTT |
| | | | | | CTGGAACCCGATGCTCCTGA |
| | | | | | ATTC (SEQ ID NO: 33) |
| 6E2 | SFRS14 | NM_001017392.3 | FIG. 15 | KAECFKNLIV | AAGCAGAGTGCTTTAAAAAT |
| | | | (SEQ ID | KKQKSLCSGF | TTGATAGTAAAAAAGCAAAA |
| | | | NO: 59) | KEHLNEASIL | ATCTCTGTGCTCTGGTTTTAA |
| | | | | AQVSVSSSKR | GGAACATTTGAATGAGGCAA |
| | | | | VWKSWENLIS | GCATTTTAGCACAGGTTTCTG |
| | | | | SFMVWNPAH | TTTCAAGTTCAAAGAGAGTC |
| | | | | LIISIPNLEKTS | TGGAAAAGTTGGGAAAATTT |
| | | | | DLSMMSKLA | AATATCATCTTTTATGGTGTG |
| | | | | AALE (SEQ ID | GAATCCTGCCCATTTGATTAT |
| | | | | NO: 9) | TTCTATCCCAAATCTTGAAA |
| | | | | | AAACATCAGACTTATCTATG |
| | | | | | ATGTCAAAGCT (SEQ ID NO: |
| | | | | | 34) |
| 3E4 | Cox5a | NM_004255.3 | FIG. 16 | NTLVTYDMV | AACACACTTGTTACCTATGA |
| | | | (SEQ ID | PEPKIIDAALR | TATGGTTCCAGAGCCCAAAA |
| | | | NO: 60) | ACRRLNDFAS | TCATTGATGCTGCTTTGCGGG |
| | | | | TVRILEVVKD | CATGCAGACGGTTAAATGAT |
| | | | | KAGPHKEIYP | TTTGCTAGTACAGTTCGTATC |
| | | | | YVIQELRPTL | CTAGAGGTTGTTAAGGACAA |
| | | | | NELGISTPEEL | AGCAGGACCTCATAAGGAAA |
| | | | | GLDKV | TCTACCCCTATGTCATCCAGG |
| | | | | (SEQ ID NO: | AACTTAGACCAACTTTAAAT |
| | | | | 10) | GAACTGGGAATCTCCACTCC |
| | | | | | GGAGGAACTGGGCCTTGACA |
| | | | | | AAGTGTAACCGCATAATAAA |
| | | | | | AGGGAAATGAGTTTGAACTG |
| | | | | | (SEQ ID NO: 35) |
| 18D3 | TIMP2 | NM_003255.4 | FIG. 17 | CSKHSSLLLFS | ATGTTCTAAGCACAGCTCTCT |
| | | | (SEQ ID | SCKQLKIFKIK | TCTCCTATTTTCATCCTGCAA |
| | | | NO: 61) | FTL (SEQ ID | GCAACTCAAAATATTTAAAA |
| | | | | NO: 11) | TAAAGTTTACATTGTAGTTAT |
| | | | | | TTTCAAATCTTTGCTTGATAA |
| | | | | | GTATTAAGAAATATTGGACT |
| | | | | | TGCTGCCGTAATTTAAAGCT |
| | | | | | CTGTTGATTTTGTTTCCGTTT |
| | | | | | GGATTTTTGGGGGAGGGGAG |
| | | | | | CACTGTGTTTATGCTGGAAT |
| | | | | | ATGAAGTCTGAGACCTTCGG |
| | | | | | TGCTGGGAACACACAAGAGT |
| | | | | | TGTTGAAAGTTGACAAGCAG |
| | | | | | ACTGCGCATGTCTCTGATGCT |
| | | | | | TTGTATCATTCTTGAGCAATC |
| | | | | | GCTCGGTCCGTGGACAATAA |
| | | | | | ACAGTATTATCAAAGAGAAA |
| | | | | | AAAAA (SEQ ID NO: 39) |
| 8E10 | BRMSL1 | NM_032352.3 | FIG. 18 | APRTRTLRAR | TCGTCGAGGCTCCTGCTCCTG |
| | | | (SEQ ID | RSPRMEIAQK | TGACTCTCGAGCAGCCAGAG |
| | | | NO: 62) | WMMKTVKEE | GCTCCTACCTCTATCGAGTCT |
| | | | | EWNVWMKCP | TTACCTACTACTTCTGACACT |
| | | | | ILKNSLPIS | TTCTTCTTCTTACCTTACAAA |
| | | | | KINFIKND | CCTACTTTACAGGTTAGAAC |
| | | | | (SEQ ID NO: | TTTTTGTCAAATGGCTAGAGT |
| | | | | 12) | TTCTAGTTGAAATATTTCTTG |
| | | | | | CTAATTCAGTCCACCTACGTT |
| | | | | | TTGATGTTCTTCAGTATCGAC |
| | | | | | CTTTTCGTGGTCTTATGAACC |
| | | | | | TTGGCGACCGTTGAAATGTC |
| | | | | | CTTTTATACGTTTAAGCATGT |
| | | | | | TTCCATCGTCCTTAGATATCT |
| | | | | | CTCGAGACGAATCTTAGACA |
| | | | | | TTTCTTGTTTATACTTACACT |
| | | | | | TTAAGTTCGAA (SEQ ID NO: |
| | | | | | 37) |
| 15F1 | RP3- | NM_005381.2 | FIG. 19 | LVSILLTKTIY | TTACTGTTACCTGATCAATGA |
| | 323M22 | | (SEQ ID | (SEQ ID NO: | CAGAGCCTTCTGAGGACATT |
| | (Nudeolin) | | NO: 63) | 13) | CCAAGACAGTATACAGTCCT |
| | | | | | GTGGTCTCCTTGGAAATCCG |
| | | | | | TCTAGTTAACATTTCAAGGG |
| | | | | | CAATACCGTGTTGGTTTTGAC |
| | | | | | TGGATATTCATATAAACTTTT |
| | | | | | TAAAGAGTTGAGTGATAGAG |
| | | | | | CTAACCCTTATCTGTAAGTTT |
| | | | | | TGAATTTATATTGTTTCATCC |
| | | | | | CATGTACAAAACCATTTTTTC |
| | | | | | CTACAAATAGTTTGGGTTTTG |
| | | | | | TTGTTGTTTCTTTTTTTTGTTT |
| | | | | | TGTTTTTGTTTTTTTTTTTTTT |
| | | | | | GCGTTCGTGGGGTTGTAAAA |
| | | | | | GAAAAGAAAGCAGAATGTTT |
| | | | | | TATCATGGTTTTTGCTTCAGC |
| | | | | | GGCTTTAGGACAAATTAAAA |
| | | | | | G (SEQ ID NO: 38) |
| 36C4 | Homo | AC128709 | FIG. 20 | LICISLMA | CCTGGGCAGTGATTAGGTCA |
| | sapiens | (Homo | (SEQ ID | NDVEHLF | TAAAGGTGGAGTCCTCATGG |
| | chromosome 3 | sapiens 3 | NO: 64) | MFICHLS | ATGGGATTAGTGTCTTTATA |
| | genomic | BAC RP13- | | (SEQ ID NO: | AAAGAGACCTTTGCCATGTG |
| | contigs | 61613) | | 14) | AGGTTACAGTGAGAAGACAT |
| | (NKX3-1) | | | | CTGTCTATGAAGAAAGTGGG |
| | | | | | CCCTCACCAAACACAGTCTG |
| | | | | | CTGGCACTTTGCACTTCAACT |
| | | | | | CCCCAGCTTCCAGAACTGTA |
| | | | | | AGGAATATAAGTCTGTTGTT |
| | | | | | GGTAAGCCACCCGGTCTATG |
| | | | | | ATATTTTGTTATAGCAGCCCA |
| | | | | | AACAGACTAAGACAGGTGAC |
| | | | | | AAATAAACATGAAAAGATGT |
| | | | | | TCAACATCATTAGCCATTAG |
| | | | | | GGAAATGCAGATTAAAA |
| | | | | | (SEQ ID NO: 39) |
| 5D11 | CSNK | NM_001896.2 | FIG. 21 | SSCSEYNVRV | TCATCCTGCTCGGAGTACAA |
| | 2A2 | | (SEQ ID | ASRYFKGPEL | TGTTCGTGTAGCCTCAAGGT |
| | | | NO: 65) | LVDYQMYDY | ACTTCAAGGGACCAGAGCTC |
| | | | | SLDMWSLGC | CTCGTGGACTATCAGATGTA |
| | | | | MLASMIFRRE | TGATTATAGCTTGGACATGT |
| | | | | PFFHGQDNYD | GGAGTTTGGGCTGTATGTTA |
| | | | | QLVRIAKVLG | GCAAGCATGATCTTTCGAAG |
| | | | | TEELYGYLKK | GGAACCATTCTTCCATGGAC |
| | | | | YHIDLDPHFN | AGGACAACTATGACCAGCTT |
| | | | | DILGQHSRKR | GTTCGCATTGCCAAGGTTCT |
| | | | | WENLSIVRTD | GGGTACAGAAGAACTGTATG |
| | | | | TLSALRP | GGTATCTGAAGAAGTATCAC |
| | | | | (SEQ ID NO: | ATAGACCTAGATCCACACTT |
| | | | | 15) | CAACGATATCCTGGGACAAC |
| | | | | | ATTCACGGAAACGCTGGGAA |
| | | | | | AACTTATCCATAGTGAGAAC |
| | | | | | AGACACCTTGTCAGCCCTGA |
| | | | | | GGCCCTAGATCTTCTGGACA |
| | | | | | AACTTCTGCGATACGACCAT |
| | | | | | CAACAGAGACTGACTGCCAA |
| | | | | | AGAGGCCATGGAGCACCCAT |
| | | | | | ACTTCTACCCTGTGGTGAAG |
| | | | | | GAGCAGTCCCAGCCTTGTGC |
| | | | | | AGACAATGCTGTGCTTTCCA |
| | | | | | GTGGTCTCACGGCAGCACGA |
| | | | | | TGAAGACTGGAAAGCGACGG |
| | | | | | GT (SEQ ID NO: 40) |
| 5F8 | Desmocollin 3 | NG_016782.1 | FIG. 22 | SAFRGYLANN | TAAGCTTTCATCTTCCCCAAC |
| | | | (SEQ ID | K | CCTGATGTCTTCCTATTCTCA |
| | | | NO: 66) | (SEQ ID NO: | CTGATCCCCCTACTGACTCA |
| | | | | 16) | GCTTCACGCTTCTTGATTATA |
| | | | | | CCTCTCTCCTGTAGAAAAGC |
| | | | | | CTTGGCTGGCTCTCCTTTAGG |
| | | | | | ATGAGAATAAATCCGAAATC |
| | | | | | CTTAGTGTAGCATTTAGAAG |
| | | | | | TCCTATCTCCCACTTGTTTCT |
| | | | | | TAATATTCTCTTCTCTAACAC |
| | | | | | CGAACTTGTTTCAAGCCTCTT |
| | | | | | TTCCAACACATGATTTCTTCT |
| | | | | | ATTCTAAATCAATTTATTTAT |
| | | | | | TATTTGCTAAATAGCCCCTA |
| | | | | | AAC |
| | | | | | (SEQ ID NO: 41) |
| 3D10 | RPL34 | NM_033625.2 | FIG. 23 | QARLFIFITQK | TTCTCGAGTGCGGCCGCAGC |
| | | | (SEQ ID | SFIFLFSFLTLC | TTGGGTATGGAGACATATCA |
| | | | NO: 67) | LCLQHFHNDF | TATAAGTAATGCTAGGGTCN |
| | | | | LLLDKESTLD | GTGGTAGGAAGTTTTTTCAT |
| | | | | PVTNTFSTHG | AGGAGGTGTATGAGTTGGTC |
| | | | | TKTLLLTSLFL | GTAGCGGAATCGGGGGTATG |
| | | | | (SEQ ID NO: | CTGTTCGAATTCATAAGAAC |
| | | | | 17) | AGGGAGGTTAGAAGTAGGGT |
| | | | | | CTTGGTTCCATGTGTGCTAAA |
| | | | | | TGTGTTCGTGACAGGATCAA |
| | | | | | GCGTGCTTTCCTTATCGAGG |
| | | | | | AGCAGAAAATCGTTGTGAAA |
| | | | | | GTGTTGAAGGCACAAGCACA |
| | | | | | GAGTCAGAAAGCTAAATAAA |
| | | | | | AAAATGAAACTTTTTTGAGT |
| | | | | | AATAAAAATGAAAAGACGC |
| | | | | | GCTTGA (SEQ ID NO: 42) |
| 3D11 | Chromosome | AC117381.5 | FIG. 24 | VSTFLSRVGR | GTTTCCACATTCTTGTCAAGG |
| | 3 UTR | (Homo | (SEQ ID | VSLLNFLPF | GTTGGTAGGGTCAGTCTTTTA |
| | region | sapiens 3 | NO: 68) | (SEQ ID NO: | AATTTCTTGCCATTTTAGTGA |
| | ropporin/ | BAC RP11- | | 18) | CTGTGCATTGGTATTTCATTG |
| | RhoEGF | 783D3) | | | TGGTTTATTTGCATGATGACT |
| | | | | | AATGCTCAACACCAACTAAT |
| | | | | | CATGTTGAGTATTTTTAATGT |
| | | | | | GCTTATTTGCCACTCATATAT |
| | | | | | CTTCTTTGATGAAGTGTCTCT |
| | | | | | TCAAATATTTTGCCCATTTAA |
| | | | | | AAACTGTATTGATTCTTATTA |
| | | | | | TTGAATTGCAATAATTCTTTC |
| | | | | | TATCCGGATATATATCCTTTG |
| | | | | | CCAGATATGTGTATTACAAA |
| | | | | | TGTTTTCTCCTAGCCTTCCAC |
| | | | | | CTCAGCCTCCCAAGTAGCTG |
| | | | | | GGAATGCAGGTGTGCACCAC |
| | | | | | CACTCCAGGGTTTTTTGTTGT |
| | | | | | TGTTGTTGTTGTTTTTCTGTA |
| | | | | | GAGACAGGGTCTTGCCATGC |
| | | | | | TGCCGAGGCTGCTCTCAAAC |
| | | | | | TCCTGGGATCAAGAAATCCT |
| | | | | | CCTGCCTCGGCCTCCCAAAG |
| | | | | | TGCTGACATTACAAGCATGA |
| | | | | | GCCACTGTGCCTGGCTAACT |
| | | | | | TTTCATCTTTTAAAGTAGTGT |
| | | | | | CTTGCAAAGAACAACATTTT |
| | | | | | AATGAAGTCCATTTATCAAC |
| | | | | | TTTTTGATTCATTGTCCATGC |
| | | | | | TTTTTGCATAATAAGAAATCT |
| | | | | | TTGCCTGCCTCAAAATTGCA |
| | | | | | AAGCTT |
| | | | | | (SEQ ID NO: 43) |
| 7A9 | AURK | NM_001127230.1; | FIG. 25 | AARLGPSLEC | CGGCCGCCCGCCTTGGCCCG |
| | AIP1 | NM_001127229.1; | (SEQ ID | WAAGSAGPF | TCTCTGGAGTGCTGGGCAGC |
| | | NM_017900.2 | NO: 69) | TAHRRPAQV | CGGGTCTGCGGGCCCCTTTA |
| | | (transcript | | GRPLSLARGP | CAGCACATCGCCGGCCGGCC |
| | | variants) | | SWSWRRCWS | CAGGTAGGGCGGCCTCTCTC |
| | | | | PGRCPSAPWR | CCTCGCAAGGGGGCCCAGCT |
| | | | | AGSRPAASCP | GGAGCTGGAGGAGATGCTGG |
| | | | | DWIPGPQGL | TCCCCAGGAAGATGTCCGTC |
| | | | | WLHRNPTSVR | AGCCCCCTGGAGAGCTGGCT |
| | | | | PAR | CACGGCCCGCTGCTTCCTGC |
| | | | | (SEQ ID NO: | CCAGACTGGATACCGGGACC |
| | | | | 19) | GCAGGGACTGTGGCTCCACC |
| | | | | | GCAATCCTACCAGTGTCCGC |
| | | | | | CCAGCCAGATAGGGGAAGG |
| | | | | | GGCCGAGCAGGGGGATGAA |
| | | | | | GGCGTCGCGGATGCGCCTCA |
| | | | | | AATTCAGTGCAAAAACGTGC |
| | | | | | TGAAGATCCGCCGGCGGAAG |
| | | | | | ATGAA |
| | | | | | (SEQ ID NO: 44) |
| 3C4 | 5′- | BC011652.2 | FIG. 26 | GGGRGAGGG | ATCACAAATAGGACAATACT |
| | UTR | | (SEQ ID | RGAGAGGGR | TGCTGGTCTCCAGGTAACGA |
| | BMI1 | | NO: 70) | PEAA (SEQ ID | ACAATACACGTTTTACAGAA |
| | | | | NO: 20) | GGAATGTAGACATTCTATTA |
| | | | | | TGGTTGTGGCATCAATGAAG |
| | | | | | TACCCTCCACAAAGCACACA |
| | | | | | CATCAGGTGGGGATTTAGCT |
| | | | | | CAGTGATCTTGATTCTCGTTG |
| | | | | | TTCGATGCATTTCTGCTTGAT |
| | | | | | AAAAAATCCCGGAAAGAGC |
| | | | | | AGCCGGCGCGAGGCGATCGA |
| | | | | | AGCGGGCGGAAAAGACAAT |
| | | | | | GAAAGTTAAAAGTCGTTCAG |
| | | | | | CAGAAAATGAATGCGAGCCA |
| | | | | | AGCGGCCATCTTGAAGCGAG |
| | | | | | CTGCAGACGCCGCTGTCAAT |
| | | | | | GGGCAACCAGCGCGGCCCCG |
| | | | | | AGCAGCCGCGGCCGCCACGC |
| | | | | | TCGTCTCATGCCGCCTCCGGC |
| | | | | | CGGCCTCCTCCTGCTCCGGC |
| | | | | | GCCTCGGCCTCCTCCGGCGC |
| | | | | | CTCGGCCTCCTCCTCCTCCGC |
| | | | | | CTCCGCCTCGACCTCCAACG |
| | | | | | CCTCCTCCTCCGGGGCCTCCT |
| | | | | | CCTCCTCCTCCTCGGC (SEQ |
| | | | | | ID NO: 45) |
| 12B2 | 5′- | BC011652.2 | FIG. 26 | QRSGRDNGD | AAGCTTATTATCTCATCATCA |
| | UTR | | (SEQ ID | VGAGAPFRLS | GTTATAATTCTCTTATCTTCA |
| | BMI1 | | NO: 70) | STSQPRRIKPI | TCTGCAACCTCTCCTCTATCT |
| | | | | APPPRAPSPEX | TCATTAGAGCCATTGGCAGC |
| | | | | GAGGGGGGR | ATCAGCAGAAGGATGAGCTG |
| | | | | GGGGGGPGG | CATAAAAATCCCTTCTTCTCT |
| | | | | GGVGGRGGG | TCATTTCATTTTTGAAAAGCC |
| | | | | GGGGGRGAG | CTGGAACTAATTTGTATACA |
| | | | | GGRGAGAGG | ATATCTTGGAGAGTTTTATCT |
| | | | | GRPEAA (SEQ | GACCTTATATTCAGTAGTGG |
| | | | | ID NO: 21) | TCTGGTCTTGTGAACTTGGAC |
| | | | | | ATCACAAATAGGACAATACT |
| | | | | | TGCTGGTCTCCAGGTAACGA |
| | | | | | ACAATACACGTTTTACAGAA |
| | | | | | GGAATGTAGACATTCTATTA |
| | | | | | TGGTTGTGGCATCAATGAAG |
| | | | | | TACCCTCCACAAAGCACACA |
| | | | | | CATCAGGNGGGGATTTAGCT |
| | | | | | CAGTGATCTTGATTCTCGTTG |
| | | | | | TTCGATGCATTTCTGCTTGAT |
| | | | | | AAAAAATCCCGGAAAGAGC |
| | | | | | AGCCGGCGCGAGGCGATCGA |
| | | | | | AGCGGGCGGAAAAGACAAT |
| | | | | | GAAAGTTAAAAGTCGTTCAG |
| | | | | | CAGAAAATGAATGCGAGCCA |
| | | | | | AGCGGCCATCTTGAAGCGAG |
| | | | | | CTGCAGACGCCGCTGTCAAT |
| | | | | | GGNCAACCAGCGCGGCCCCG |
| | | | | | AGCAGCCGCGGCCGCCACGC |
| | | | | | TCGTCTCATGCCGCCTCCGGC |
| | | | | | CGGCCTCCTCCTGCTCCGGC |
| | | | | | GCCTCGGCCTCCTCCGGCGC |
| | | | | | CTCGGCCTCCTCCTCCTCCGC |
| | | | | | CTCCGCCTCGACCTCCAACG |
| | | | | | CCTCCTCCTCCGCTTGAATTC |
| | | | | | GGATCCCCGAGCATCACACC |
| | | | | | TGACTGGAATACGAACAGCT |
| | | | | | CCACATNCNGT (SEQ ID NO: |
| | | | | | 46) |
| 1D10 | 5′- | BC011652.2 | FIG. 26 | GGRGGGGGG | GGAGGTCGAGGCGGAGGCG |
| | UTR- | | (SEQ ID | GGRGAGGGR | GAGGAGGAGGAGGCCGAGG |
| | BMI1 | | NO: 70) | GAGAGGGRP | CGCCGGAGGAGGCCGAGGC |
| | | | | EAA (SEQ ID | GCCGGAGCAGGAGGAGGCC |
| | | | | NO: 22) | GGCCGGAGGCGGCATGAGAC |
| | | | | | GAGCGTGGCGGCCGCGGCTG |
| | | | | | CTCGGGGCCGCGCTGGTTGN |
| | | | | | CCATTGACAGCGGCGTCTGC |
| | | | | | AGCTCGCTTCAAGATGGCCG |
| | | | | | CTTGGCTCGCATTCATTTTCT |
| | | | | | GCTGAACGACTTTTAACTTTC |
| | | | | | ATTGTCTTTTCCGCCCGCTTC |
| | | | | | GATCGCCTCGCGCCGGCTGC |
| | | | | | TCTTTCCGGGATTTTTTATCA |
| | | | | | AGCAGAAATGCATCGAACAA |
| | | | | | CGAGAATCAAGATCACTGAG |
| | | | | | CTAAATCCCCNCCTGATGTG |
| | | | | | TGTGCTTTGTGGAGGGTACTT |
| | | | | | CATTGATGCCACAACCATAA |
| | | | | | TAGAATGTCTACATTCCTTCT |
| | | | | | GTAAAACGTGTATTGTTCGTT |
| | | | | | ACCTGGAGACCAGCAAGTAT |
| | | | | | TGTCCTATTTGTGATGTCCAA |
| | | | | | GTTCACAAGACCAGACCACT |
| | | | | | ACTGAATATAAGGTCAGATA |
| | | | | | AAACTCTCCAAGATATTGTA |
| | | | | | TACAAATTAGTTCCAGGGCT |
| | | | | | TTTCAAAAATGAAATGAAGA |
| | | | | | GAAGAAGGGATTTTTATGCA |
| | | | | | GCTCATCCTTCTGCTGATGCT |
| | | | | | GCCAATGGCTCTAATGAAGA |
| | | | | | TAGAGGAGGACGGTTGCAGA |
| | | | | | TGAAGATAAGAGAATTATAA |
| | | | | | NCTGATGATGAGATAATAAG |
| | | | | | GCTTGCGGCCGCACTCGAGA |
| | | | | | AACAGT (SEQ ID NO: 47) |
| 18D2 | 5′- | BC011652.2 | FIG. 26 | GVGGRGGGG | GGCGTTGGAGGTCGAGGCGG |
| | UTR- | | (SEQ ID | GGGGRGAGG | AGGCGGAGGAGGAGGAGGC |
| | BMI1 | | NO: 70) | GRGAGAGGG | CGAGGCGCCGGAGGAGGCC |
| | | | | RPEAA (SEQ | GAGGCGCCGGAGCAGGAGG |
| | | | | ID NO: 23) | AGGCCGGCCGGAGGCGGCAT |
| | | | | | GAGACGAGCGTGGCGGCCGC |
| | | | | | GGCTGCTCGGGGCCGCGCTG |
| | | | | | GTTGNCCATTGACAGCGGCG |
| | | | | | TCTGCAGCTCGCTTCAAGAT |
| | | | | | GGCCGCTTGGCTCGCATTCA |
| | | | | | TTTTCTGCTGAACGACTTTTA |
| | | | | | ACTTTCATTGTCTTTTCCGCC |
| | | | | | CGCTTCGATCGCCTCGCGCC |
| | | | | | GGCTGCTCTTTCCGGGATTTT |
| | | | | | TTATCAAGCAGAAATGCATC |
| | | | | | GAACAACGAGAATCAAGATC |
| | | | | | ACTGAGCTAAATCCCCNCCT |
| | | | | | GATGTGTGTGCTTTGTGGAG |
| | | | | | GGTACTTCATTGATGCCACA |
| | | | | | ACCATAATAGAATGTCTACA |
| | | | | | TTCCTTCTGTAAAACGTGTAT |
| | | | | | TGTTCGTTACCTGGAGACCA |
| | | | | | GCAAGTATTGTCCTATTTGTG |
| | | | | | ATGTCCAAGTTCACAAGACC |
| | | | | | AGACCACTACTGAATATAAG |
| | | | | | GTCAGATAAAACTCTCCAAG |
| | | | | | ATATTGTATACAAATTAGTTC |
| | | | | | CAGGGCTTTTCAAAAATGAA |
| | | | | | ATGAAGAGAAGAAGGGATTT |
| | | | | | TTATGCAGCTCATCCTTCTGC |
| | | | | | TGATGCTGCCAATGGCTCTA |
| | | | | | ATGAAGATAGAGGAGGACG |
| | | | | | GTTGCAGATGAAGATAAGAG |
| | | | | | AATTATAANCTGATGATGAG |
| | | | | | ATAATAAGGCTTGCGGCCGC |
| | | | | | ACTCGAGAAACAGT (SEQ ID |
| | | | | | NO: 48) |
| 21B4 | LAMR | NM_002295.4 | FIG. 27 | REMVPRMRR | CGCGAAATGGTGCCGCGCAT |
| | 1 | | (SEQ ID | TSRASIHHIKP | GCGCCGCACCAGCCGCGCGA |
| | | | NO: 71) | TE (SEQ ID | GCATTCATCATATTAAACCG |
| | | | | NO: 24) | ACCGAA |
| | | | | | (SEQ ID NO: 49) |
| 2D4 | Contig | NT_011255.14 | FIG. 28 | LAHRPPCAEP | GGAGTTTCACTTTTGTTGCCC |
| | GRCh3 | | (SEQ ID | DPGQRMELPA | AGGATTGAGTGCAGTGCCCC |
| | 7.p2 | | NO: 72) | PVPRPRGASK | GATCTTGGCTCACTACAACC |
| | (13714 | | | PRDGTSSHCD | TCTGCCTCCTGGGTTCAAGC |
| | 51::137 | | | MPNCQHPQG | GACTCTCCTGCCTCAGTGTCC |
| | 2008) | | | PGPAGEIRSRC | TGAGTAGCTGGGATTACAGG |
| | | | | RSCWLRAVR | CGTCTGCCACCACGCCCGGC |
| | | | | CNPWLGR | TAATTTTGTATTTTTAGTAGA |
| | | | | (SEQ ID NO: | GAACAGGTTTCACTATGTTG |
| | | | | 25) | GTCAGGCTGGTCTTGAACTC |
| | | | | | CTGACCTCAGCGCATCCAGA |
| | | | | | ATTTTAGACGGGGCCCCCAG |
| | | | | | GGTGAGGTCTTGGCACCCTC |
| | | | | | CAGTAGAGAAGAAGGGACA |
| | | | | | TGGGCCATACGTGGGGTGTC |
| | | | | | CTTTCTGGGAGCCTTGCGTCC |
| | | | | | CTTACCTGCCTAGCCAGGGA |
| | | | | | TTGCACCTCACAGCACGCAG |
| | | | | | CCAGCAGGAACGGCACCGTG |
| | | | | | ATCTGATTTCACCTGCGGGC |
| | | | | | CCTGGGCCCTGGGGGTGTTG |
| | | | | | ACAATTGGGCATATCACAGT |
| | | | | | GTGAGCTAGTCCCGTCTCGG |
| | | | | | GGTTTGGAGGCTCCACGTGG |
| | | | | | CCGTGGTACAGGAGCAGGCA |
| | | | | | GTTCCATCCTCTGGCCTGGAT |
| | | | | | CAGGCTCTGCACACGGAGGC |
| | | | | | CTGTGGGCCAG |
| | | | | | (SEQ ID NO: 50) |
| 4C4 | PSA | M27274.1 | FIG. 29 | SEGRTVT | TGTGTGGGTATGAGGGTATG |
| | | | (SEQ ID | NKVSRKY | AGAGGGCCCCTCTCACTCCA |
| | | | NO: 73) | TG (SEQ ID | TTCCTTCTCCAGGACATCCCT |
| | | | | NO: 74) | CCACTCTTGGGAGACACAGA |
| | | | | | GAAGGGCTGGTTCCAGCTGG |
| | | | | | AGCTGGGAGGGGCAATTGAG |
| | | | | | GGAGGAGGAAGGAGAAGGG |
| | | | | | GGAAGGAAAACAGGGTATG |
| | | | | | GGGGAAAGGACCCTGGGGA |
| | | | | | GCGAAGTGGAGGATACAACC |
| | | | | | TTGGGCCTGCAGGCCAGGCT |
| | | | | | ACCTACCCACTTGGAAACCC |
| | | | | | ACGCCAAAGCCGCATCTACA |
| | | | | | GCTGAGCCACTCTGAGGCCT |
| | | | | | CCCCTCCCCGGCGGTCCCCA |
| | | | | | CTCAGCTCCAAAGTCTCTCTC |
| | | | | | CCTTTTCTCTCCCACACTCTA |
| | | | | | TCATCCCCCGGATTCCTCTCT |
| | | | | | ACTTGGTTCTCATTCTTCCTT |
| | | | | | TGACTTCCTGATCCTGTGTAT |
| | | | | | TTTCGGCTCACCTTGATTTGT |
| | | | | | CACTGTTCTCCCCTC (SEQ ID |
| | | | | | NO: 75) |
| 4H9 | RPSA | NC_000003.11 | FIG. 30 | GKWCHACAE | CGGGAAATGGTGCCACGCAT |
| | (minus strand) | (c394033- | (SEQ ID | LPEPASTTSNP | GCGCAGAACTTCCCGAGCCA |
| | | 39448204) | NO: 76) | LSELPCCCMG | GCATCCACCACATCAAACCC |
| | | | | WQCPHSAEE | ACTGAGTGAGCTCCCTTGTT |
| | | | | NLCYTAQW | GTTGCATGGGATGGCAATGT |
| | | | | (SEQ ID NO: | CCACATAGCGCAGAGGAGAA |
| | | | | 77) | TCTGTGTTACACAGCGCAAT |
| | | | | | GGTAGGTAGGTTAACATAAG |
| | | | | | ATGCCTCCGTGAGAGGCTGG |
| | | | | | TGGTCAGCCCTGGGGTCAGT |
| | | | | | AACCACAAGAAGCCGTGGCT |
| | | | | | CCCGGAAGGCTGCCTGGATC |
| | | | | | TGGTTAGTGAAGGTTCCAGG |
| | | | | | AGTGAAGCGGCCAGCAATTG |
| | | | | | GAGTGGCTCCAGTGGCAGCA |
| | | | | | GCAAACTTCAGCACAGCCCT |
| | | | | | CTGGCCAGTATTCCTGGAGG |
| | | | | | ATATAACACTGACATCAGCA |
| | | | | | GGGTTTTCAATGGCAACAAT |
| | | | | | TGCACGAGCTGCCAGCAGAA |
| | | | | | GCTT (SEQ ID NO: 78) |
| 40A3 | RBM6 | NC_000003.11 | FIG. 31 | LRGITKNDRN | CTCTGAGGGGCATCACCAAA |
| | (minus strand) | (c50114685-) | (SEQ ID | FNRKIHLNWI | AATGACAGGAATTTCAACAG |
| | | 50109685) | NO: 79 | SK (SEQ ID | GAAGATACATCTGAATTGGA |
| | | | | NO: 80) | TCTCGAAATAAGGAGTTTGT |
| | | | | | GTAAGAGAAAAGGAGGACA |
| | | | | | CAAGCAAGGAGACACAAAA |
| | | | | | GACAATTTGTCCAAGAGAGT |
| | | | | | AGTAGTAGAAACTGACAAAG |
| | | | | | GTAAGGCTGCTTGGTGGCCG |
| | | | | | GGTGCAGTGACTCACGCCTG |
| | | | | | TAATCCCAGCACTTTGGGAG |
| | | | | | GCCAAGGCGGGTGGATCACC |
| | | | | | TGAGGTCAGGAGTTCGAGAC |
| | | | | | CACCCTGACCAACAGGTGAA |
| | | | | | ACCCCTCTCTACTAAAAATA |
| | | | | | CAAACATTAGCCCATAGTCC |
| | | | | | CAGCTACTGGGGAGGCTGAG |
| | | | | | GCAGGAGAATCGCTTGAACC |
| | | | | | TGGGAGGCGGAGGTTGCAGT |
| | | | | | GAGCCAAGATCGTGCCATTG |
| | | | | | CACTCCAGCCTGGGCGACAG |
| | | | | | AATGAGACTGTCTCAAAACA |
| | | | | | AAAGGAAAAAAAAAA (SEQ |
| | | | | | ID NO: 81) |
| 21D10 | hypothetical | XP_353238.1 | | PASASILAGVP | CCGGCGAGCGCGAGCAATTC |
| | protein | | | MYRNEFTAW | TGGCGGGCGTGCCGATGGTA |
| | XP_353238 | | | RRMSVVYGIG | TCGAACGAATTACCGCGTGG |
| | | | | TWSVLGSLLY | TATCGCCGCATGAGCGTGGT |
| | | | | YSRTMAKSSV | GTATGGCATTGGCACCTGGA |
| | | | | DQKDGSASEV | GCGTGCTGGGCAGCCTGCTG |
| | | | | PSELSERPSLR | TATTATAGCCGCACCATGGC |
| | | | | PHSSN (SEQ | GAAAAGCAGCGTGGATCAGA |
| | | | | ID NO: 82) | AAGATGGCAGCGCGGAAGTG |
| | | | | | CCGAGCGAACTGAGCGAACG |
| | | | | | CCCGAGCCTGCGCCCGCATA |
| | | | | | GCAGCACC (SEQ ID NO: 83) |

An antibody, such as an autoantibody, to one or more of a protein, or a fragment of a protein, encoded by a gene such as listed in Table 1, or a polypeptide encoded by a UTR sequence of a gene such as one listed in Table 1, can be detected according to one or more methods described herein and used to detect a cancer, such as prostate cancer Other suitable markers can include those known in the art, such as biomarkers disclosed in U.S. patent application Ser. No. 13/050,544, U.S. patent application Ser. No. 13/072,542, and U.S. Pat. No. 7,858,323, which are hereby incorporated by reference in their entirety. Many of the proteins may have a role in various cancers, including prostate cancer. For example, the human DCHS1 protein (protocadherin-16 precursor) is believed to be a calcium-dependent cell adhesion protein found in the cell membrane of fibroblast cells. Without being bound by theory, DCHS1 is a cadherin, a class of type-1 transmembrane proteins. Cadherins typically play important roles in cellular adhesion, for example, by binding cells expressing similar cadherins to each other. Structurally, DCHS1 is thought to contain 27 cadherin repeats (extracellular calcium ion-binding domains). DCHS1 expression has been associated with certain cancers, potentially playing a role in tumor adherence (see, e.g., Sjöblom, et. al. Science, (2006) 314:268-274).

Another of the proteins, CEP164 is believed to be a centrosomal protein which binds chromatin and plays a role in the DNA damage-activated signaling cascade. It is known to interact with ataxia telangiectasia mutated (ATM) and ATM/Rad3-related (ATR) kinases which phosphorylate CEP164 upon replication stress, ultraviolet radiation (UV), and ionizing radiation (IR). CEP164 also plays a role in cell cycle regulation, specifically at the G2/M checkpoint and in nuclear division (see, e.g., Sivasubramaniam et al., Genes & Dev. (2008); 22(5):687-600). As CEP164 plays a role in genome stabilization, misregulation or mutation of this gene and/or protein can play a role in certain cancers.

In a further example, the human KBTBD6 (kelch repeat and BTB (POZ) domain containing 6) is a protein expressed in a wide variety of normal tissues. Its expression and/or misregulation has also been noted in multiple cancer types, including prostate, ovarian, kidney and lung tumors. The function of the protein is not currently known, however, the presence of the kelch repeat and BTB domain suggest that the protein is involved in protein-protein interactions and actin filament organization.

Certain ribosomal proteins, such as RPS19 and RPL34 have also been associated with certain cancers. RPS19 (ribosomal protein S19) encodes a ribosomal protein that is a component of the 40S subunit. Located in the cytoplasm as part of the ribosomal complex, mutations in this gene are associated with Diamond-Blackfan anemia, suggesting a non-ribosomal function for the protein in erythropoietic differentiation. RPS19 protein is also known to interact with fibroblast growth factor-2 (see, e.g., Soulet et al., Biochem. Biophys. Res. Commun. (2001); 289:591-596). Increased expression of RPS19 has been associated with some cancers, but the role of RPS19 in cancer development is unknown. RPL34 (60S Ribosomal protein L34) is a ribosomal protein that is a component of the 60S subunit and is located in the cytoplasm. Expression of the gene encoding the RPL34 protein is known to be regulated by c-MYC and has been shown to have increased expression in primary invasive and metastatic breast cancer cells and colorectal cancer cells (see, e.g., Zucchi et al., Proc. Nat'l Acad. Sci., (2004); 101:18147-18152; Sjöblom, et. al. Science, (2006) 314:268-274).

Certain nucleic acid-binding proteins, such as RMB6 and HEMK1 have also been associated with certain cancers when misregulated and/or mutated. RBM6 (RNA binding protein 6) is a cytosolic protein that binds to poly-G homopolymers in vitro, but its function in vivo is not currently known. The protein thought to be phosphorylated (potentially by ATM or ATR) in its active form. The gene encoding the protein, without being bound by theory, is located in a portion of the genome, modifications of which are associated with cancerous transformation, such as lung carcinomas. Additionally, translocations of the gene which result in aberrant fusion proteins have been reported to be associated with cancer cells (see, e.g., Gu et al., Blood, (2007); 110:323-333). The human HEMK1 (HEMK methyltransferase family protein 1) protein is an S-adenosylmethionine-dependent methyltransferase and is also thought to bind nucleic acids. HEMK1 is considered a tumor-suppressor, misregulation of which is associated with various cancers, including prostate cancer, pancreatic cancer and liver cancer (see, e.g., U.S. Pat. App. Pub. No. 2008/0213791).

Antigens associated with cancers that give rise to autoantibodies have been described in colon, breast, lung, ovary, or head and neck cancers. These antigens are incorporated herein by reference in their entirety: Scanlan et al. Characterization of human colon cancer antigens recognized by autologous antibodies. Intl J Cancer 1998; 76:652-8; Disis et al. Existent T-cell and antibody immunity to HER-2/neu protein in patients with breast cancer. Cancer Res 1994; 54:16-20; Diesinger et al. Toward a more complete recognition of immunoreactive antigens in squamous cell lung carcinoma. Int J Cancer 2002; 102:372-8; Chatterjee et al. Diagnostic markers of ovarian cancer by high-throughput antigen cloning and detection on arrays. Cancer Res 2006; 66:1181-90; Lin et al., Cancer Epidemiol. Biomarkers Prev. 2007 November; 16(11):2396-405.

Thus one or more polypeptide probes, such as a fragment of a protein encoded by a gene, or a polypeptide encoded by a sequence of a UTR region of a gene, such as a gene listed in Table 1, can be used to detect one or more antibodies, such as autoantibodies, from a sample from a subject. In one embodiment, a polypeptide probe is a fragment of a protein encoded by a gene, or a fragment encoded by a sequence of a UTR region of a gene. In one embodiment, the gene can be alpha-2 glycoprotein1 (AZGP1), DCHS1 (protocadherin-16 precursor), Homo sapiens family with sequence similarity 53, member B (FAM53B), mitogen-activated protein kinase kinase kinase 9 (MAPKKK9), Deaminase Domain, ADP-ribosylation factor 6 (ARF6), Centrosomal Protein 164 kDa (CEP 164), ADAM metallopeptidase domain 9 (ADAMS), splicing factor, arginine/serine-rich 14 (SFRS14), cytochrome c oxidase subunit Va (Cox5a), TIMP metallopeptidase inhibitor 2 (TIMP2), breast cancer metastasis-suppressor 1-like (BRMSL1), RP3-323M22 (Nucleolin), Homo sapiens chromosome 3 genomic contigs (NKX3-1), casein kinase 2, alpha prime polypeptide (CSNK2A2), Desmocollin 3 (DSC3), ribosomal protein L34 (RPL34), Chromosome 3 UTR region ropporin/RhoEGF (3′ UTR Ropporin), aurora kinase A interacting protein 1 (AURKAIP1), 5′-UTR BMI1, ribosomal protein SA (LAMR1), Contig GRCh37.p2 (1371451::1372008), PSA, RPSA (minus strand), RBM6 (minus strand), or hypothetical protein XP_{—}353238. Other suitable markers can include those known in the art, such as biomarkers disclosed in U.S. patent application Ser. No. 13/050,544, U.S. patent application Ser. No. 13/072,542, and U.S. Pat. No. 7,858,323, which are hereby incorporated by reference in their entirety. A polypeptide probe can comprise a peptide sequence, or fragment thereof, such as those listed in Table 1. In one embodiment, a polypeptide probe comprises SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 74, SEQ ID NO: 77, SEQ ID NO: 80, or SEQ ID NO: 82, or a fragment thereof. In another embodiment, a polypeptide probe comprises a polypeptide encoded by SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 75, SEQ ID NO: 78, SEQ ID NO: 81, OR SEQ ID NO: 83, or a fragment thereof. In another embodiment, a polypeptide probe comprises a polypeptide encoded by SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65 SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 76, or SEQ ID NO: 79, or a fragment thereof.

Probe Presentation

In one embodiment, a phage is used as a vehicle for presenting probes to antibodies or autoantibodies. The presentation of a probe can be accomplished by expressing the probe on the surface of the phage or chemically coupling the probe to a protein on the surface of the phage. Expression on the surface can be accomplished by expressing the probe as part of a phage surface protein, such as a capsid protein. Detailed methods and compositions for coupling a probe to a phage or coupling a phage to a particle is described herein.

Any phage can be used as a presentation vehicle. A phage useful for methods and compositions described herein includes, but is not limited to, MS2 phage, T2 phage, T4 phage, lambda phage, T12 phage, R17 phage, M13 phage, G4 phage, P1 phage, P2 phage, P4 phage, Phi X 174 phage, N4 phage, Phi6 phage, Phi29 phage, and 186 phage. In one embodiment, MS2 phage is used as a vehicle to present a probe. In another embodiment, M13 phage is used as the vehicle. In another embodiment, a T7 phage is used.

In another embodiment, a virus-like particle (VLP) can be also be used as a presentation vehicle. A VLP contains one or more proteins from a virus, optionally combined or formulated with a phospholipid. A VLP is typically not pathogenic, incapable of replication, and does not contain any of the native viral genome. A virus-like particle can self-assemble when L1, the major capsid protein of human and animal papillomaviruses, is expressed in yeast, insect cells, mammalian cells or bacteria (, see Schiller and Rodent, in Papillomavirus Reviews: Current Research on Papillomaviruses; Lacey, ed. Leeds, UK: Leeds Medical Information, pp 101 12 (1996)). Morphologically indistinct VLPs can also be produced by expressing a combination of the L1 and L2 capsid proteins. VLPs are composed of 72 pentamers of L1 in a T=7 icosahedral structure (Baker et al., Bioplzys. J. 60(6): 1445 56 (1991)). VLPs are morphologically similar to authentic virions and are capable of inducing high titres of neutralizing antibodies upon administration into an animal.

The viral protein components of a VLP can be recombinantly produced or isolated from a virus. As a presentation vehicle, a probe can be coupled to a viral protein of a VLP. A viral protein useful for a method or composition described herein include, but is not limited to, a polypeptide derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ phage (such as coat proteins), GA-phage, fr-phage, AP205 phage, and Ty (such as retrotransposon Ty protein p1).

Particle

In one embodiment, a particle is coupled to a phage, wherein the phage is coupled or linked to a probe. A particle can carry information about the nature of probe and the phage. In one embodiment, the information is encoded to a particle as a form of identification. In one embodiment, identifying information is pre-assigned to the description of probes and phages, enabling detection of identifying information to be readily related to the description. Encoding can be accomplished by using a material including, but not limiting, a signal that is magnetic, isotopic, luminescent, fluorescent, or a combination thereof. For example, a particular fluorescent signal can be assigned to a particular particle. Conversely, a pool of particles can be randomly labeled with a variety of distinguishable fluorescent signals and then sorted according to differences in fluorescent signal. A fluorescent signal can be a signal from a particular fluorophore or a mix of two or more fluorophores having distinct emission spectra.

In one embodiment, a particle is coupled to a single phage. In one embodiment, a phage coupled to a particle comprises a single probe. In another embodiment, a phage coupled to a particle comprises multiple probes. In another embodiment, a particle is coupled to a plurality of phages, such as at least 2, 3, 4, 5, 6, 7, 8, 9, or 10 phages. Each of the plurality of phages may comprise the same probe or different probe as another phage coupled to a particle. In one embodiment, a subset of the plurality of phages comprises a different probe that is of the same type of probe as another subset of the plurality. For example, one subset of phages can comprise a polypeptide probe specific for one antibody and a second subset comprises a polypeptide probe specific for a second antibody. In another embodiment, a subset of the plurality of phages comprises a probe that is of a different type of probe in another subset of the plurality. For example, one subset of phages can comprise a polypeptide probe and a second subset a nucleic acid probe.

A particle provides a platform on which a phage can be coupled to. In one embodiment, a particle provides a chemical and/or physical element common to a plurality of phage-particle complexes. In one embodiment, the phage-particle complex is used in purification, separation, fractionation, partition, precipitation sorting or grouping of one or more biological molecules. For example, a particle made of glass can be precipitated, and thus separating an antibody or autoantibody bound onto the particle out of a sample.

Any material capable of carrying information, providing a solid platform, and capable of providing a common element can be used to form a probe-phage-particle complex. A particle can be made of any chemically and biologically inert material capable of carrying information. A particle can be in a form of a bead, a sphere, or a granule. In one embodiment, the particle is a microsphere. A particle can be porous or non-porous. Various polymeric materials can be used to manufacture a particle. For example, biologically inert material that produces enough strength as a particle for a phage can be employed to manufacture a particle. Polymers which can be used include, but are not limited to, the following: polystyrene; poly(tetra)-fluoroethylene (PTFE); polyvinylidenedifluoride; polycarbonate; polymethylmethacrylate; polyvinylethylene; polyethyleneimine; poly(etherether)ketone; polyoxymethylene (POM); polyvinylphenol; polylactides; polymethacrylimide (PMI); polyatkenesulfone (PAS); polypropylene; polyethylene; polyhydroxyethylmethacrylate (HEMA); polydimethyl-siloxane; polyacrylamide; polyimide; and block-copolymers. The particle can be can be polystyrene, brominated polystyrene, polyacrylic acid, polyacrylonitrile, polyacrylamide, polyacrolein, polydimethylsiloxane, polybutadiene, polyisoprene, polyurethane, polyvinyl acetate, polyvinylchloride, polyvinylpyridine, polyvinylbenzylchloride, polyvinyltoluene, polyvinylidene chloride, polydivinylbenzene, polyglycidylmethacrylate, polymethylmethacrylate, or copolymers, blends, composites, or combination thereof. In one embodiment, the microsphere comprises polystyrene.

The particle can have a diameter of between about 1 nm-1000 μm, 1 nm-500 μm, 5 nm-500 μm, or 10 nm-100 μm. In one embodiment, the particle has a diameter of between about 10 nm and 100 μm. In yet another embodiment, the particle has a diameter of less than about 1000 μm, 500 μm, 400 μm, 300 μm, 200 μm, or 100 μm. In one embodiment, the particle has a diameter of greater than or less than about 10 μn, 9 μm, 8 μm, 7 μm, 6 μm, 5 μm, 4 μm, 3 μm, 2 μm, or 1 μm. In one embodiment, the particle is a microsphere wherein the microsphere comprises identification information encoded in microsphere size. The microsphere can have a diameter of between about 1 nm-1000 μm, 1 nm-500 μm, 5 nm-500 μm, or 10 nm-100 μm. In one embodiment, the microsphere has a diameter of between about 10 nm and 100 μm. In yet another embodiment, the microsphere has a diameter of less than about 1000 μm, 500 μm, 400 μm, 300 μm, 200 μm, or 100 μm. In one embodiment, the microsphere has a diameter of greater than or less than about 10 μm, 9 μm, 8 μm, 7 μm, 6 μm, 5 μm, 4 μm, 3 μm, 2 μm, or 1 μm.

In one embodiment, the microsphere comprises identification information, wherein the identification information comprises a fluorescent signal. In one embodiment, the particle is labeled or stained with more than one dye, such as at least 2, 3, 4, 5, 6, 7, 8, 9, or 10 different dyes. In one embodiment, the particle is labeled or stained with two dyes. In another embodiment, the two dyes are hydrophobic. In another embodiment, the two dyes are fluorescent dyes, such as squaric acid-based dyes. In yet another embodiment, the squaric acid-based dyes are selected from cyclobutenedione derivatives, symmetrical and unsymmetrical squaraines, substituted cephalosporin compounds, fluorinated squaraine compositions, alkylalkoxy squaraines, or squarylium compounds. In another embodiment, the squaric acid-based dyes are selected from a red fluorescent dye and an orange fluorescent dye, such as the red fluorescent dye comprising 1,3-bis(1,3-dihydro-1,3,3-trimethyl-2H-indol-2-ylidene)methyl]-2,4-dihydroxycyclobutenediylium, bis(inner salt) and the orange fluorescent dye comprising 2-(3,5-dimethylpyrrol-2-yl)-4-(3,5-dimethyl-2H-pyrrol-2-ylidene)-3-hydroxy-2-cyclobuten-1-one. In one embodiment, the microsphere is a Luminex® bead or microsphere.

Producing a Phage-Particle Complex

In one embodiment, a probe is expressed as part of the phage. In one aspect, a probe is expressed as part of the phage. A part of the phage can be any place on the phage that is accessible to antibodies or autoantibodies. In one embodiment, the part is surface protein of a phage. In another embodiment, the surface protein is a capsid protein. To express probe as part of a phage, the DNA sequence of a polypeptide probe is inserted into genes encoding for various capsid proteins of a phage such as a T7 phage. The insertion, without being bound by theory, is expressed as part of the capsid protein and the polypeptide probe is displayed on the outside of the assembled phage. Infection of a host cell with the recombinant phage will produce a population of phage displaying the inserted polypeptide sequence, which can be used to detect antibodies that are produced by a subject. A host can be any cell type which is capable of infection by the phage. In one embodiment, the host is a bacteria. In another embodiment, the bacteria is Escherichia coli (E. coli). In one embodiment, a linker sequence of nucleotides is included at the 5′, 3′, or both ends of the inserted sequence. The linker sequence, when expressed, generates a spacer sequence of polypeptide where it is desirable in to add rotational freedom and reduce steric limitations on the displayed polypeptide probe. The linker should be of an appropriate length to allow the polypeptide probe to interact freely with antibodies in a sample. In one embodiment, the linker sequence of nucleotides encodes 1, 2, 3, 4, 5, or more glycine residues. In another embodiment, a probe is chemically coupled to the phage. In one aspect, a probe is chemically modified for coupling to a phage. Chemical modification can be a modification that leads to covalent bonding. In one embodiment, the N-terminal of a probe is modified to covalently attach to a phage. In another embodiment, the C-terminal of a probe is modified to covalently attach to a phage. In another embodiment, a side chain of an amino acid is modified to covalently attach to a phage. The modification can be chemical modification or incorporation of unusual amino acids during the synthesis of a polypeptide probe. Conversely, the phage, or a VLP can be chemically modified. In one embodiment, a capsid protein of a phage is modified to covalently attach to one or more probes.

For chemical coupling, various coupling chemistry are known in the art. For example, N-terminal or side-chain protection can be used for the synthesis of N-terminal modified polypeptide probe. Protecting agents for an amino group include, but are not limited to, acyl type protecting groups (e.g., formyl, trifluoroacetyl, acetyl), aromatic urethane type protecting groups (e.g. benzyloxycarboyl (Cbz) and substituted Cbz), aliphatic urethane protecting groups (e.g., t-butyloxycarbonyl (Boc), isopropyloxycarbonyl, cyclohexyloxycarbonyl) and alkyl type protecting groups (e.g., benzyl, triphenylmethyl). Protecting agents for side chain includes, but are not limited to, The side chain protecting groups for Tyr include tetrahydropyranyl, tert-butyl, trityl, benzyl, Cbz, Z—Br-Cbz, and 2,5-dichlorobenzyl. The side chain protecting groups for Asp include benzyl, 2,6-dichlorobenzyl, methyl, ethyl, and cyclohexyl. The side chain protecting groups for Thr and Ser include acetyl, benzoyl, trityl, tetrahydropyranyl, benzyl, 2,6-dichlorobenzyl, and Cbz. The side chain protecting group for Thr and Ser is benzyl. The side chain protecting groups for Arg include nitro, Tosyl (Tos), Cbz, adamantyloxycarbonyl mesitoylsulfonyl (Mts), and Boc. The side chain protecting groups for Lys include Cbz, 2-chlorobenzyloxycarbonyl (2-Cl-Cbz), 2-bromobenzyloxycarbonyl (2-BrCbz), Tos, and Boc. In one embodiment, coupling is performed by activating a microsphere in Sulfo-NHS (diluted in dH₂O), EDC (diluted in dH₂O) and incubating in MES, pH 5.0.

A phage coupled or linked to a probe can then be linked or coupled to a particle by a variety of methods. For example, a phage can be chemically coupled to a particle. Chemical coupling can be achieved through covalent bonding. The coupling can be mediated by methods such as streptavidin-biotin coupling. The coupling can also be mediated by using an N-hydroxysulfosuccinimide enhanced carbodiimide-mediated coupling reaction.

The successful coupling of the phage to the particle can depend upon the coupling reaction conditions. The pH of the coupling reaction is a parameter that can be modified; for example, to ensure the stability of the phage-particle complex. The pH of the reaction conditions can be between about 1 and 14; for example, 1-14, 1-13, 1-12, 1-11, 1-10, 1-9, 1-8, 1-7, 1-6, 1-5, 1-4, 1-3, 1-2, 2-14, 2-13, 2-12, 2-11, 2-10, 2-9, 2-8, 2-7, 2-6, 2-5, 2-4, 2-3, 3-14, 3-13, 3-1, 2, 3-11, 3-10, 3-9, 3-8, 3-7, 3-6, 3-5, 3-4, 4-14, 4-13, 4-12, 4-11, 4-10, 4-9, 4-8, 4-7, 4-6, 4-5, 5-14, 5-13, 5-12, 5-11, 5-10, 5-9, 5-8, 5-7, 5-6, 6-14, 6-13, 6-12, 6-11, 6-10, 6-9, 6-8, 6-7, 7-14, 7-13, 7-12, 7-11, 7-10, 7-9, 7-8, 8-14, 8-13, 8-12, 8-11, 8-10, 8-9, 9-14, 9-13, 9-12, 9-11, 9-10, 10-14, 10-13, 10-12, 10-11, 11-14, 11-13, 11-12, 12-14, 12-13, 13-14, or any included interval. The pH of the coupling reaction can be about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, or 14. In one embodiment, the pH of the coupling reaction is between about 4 and 6. In one embodiment, the pH of the coupling reaction is between about 6 and 8. In one embodiment, the pH of the coupling reaction is about 7.2. In one embodiment, the pH of the coupling reaction is about 5.

The pH of the coupling reaction can be controlled using a buffer. Any suitable buffer can be utilized according to the methods disclosed herein. The choice of a buffer to use can be made in light of the desired pH of the coupling reaction. A buffer can be a mixture of chemicals; for example: hydrochloric acid and sodium citrate, citric acid and sodium citrate, acetic acid and sodium acetate, dipotassium phosphate (K₂HPO₄) and monopotassium phosphate (KH₂PO₄), disodium phosphate (Na₂HPO₄) and monosodium phosphate (NaH₂PO₄), borax and sodium hydroxide, disodium phosphate and citric acid, etc. A buffer used in the coupling reaction can comprise TAPS buffer (3-{[tris(hydroxymethyl)methyl]amino}propanesulfonic acid), Bicine buffer (N,N-bis(2-hydroxyethyl)glycine), Tris buffer (tris(hydroxymethyl)methylamine), Tricine buffer (N-tris(hydroxymethyl)methylglycine), TAPSO buffer (3-[N-Tris(hydroxymethyl)methylamino]-2-hydroxypropanesulfonic Acid), HEPES buffer (4-2-hydroxyethyl-1-piperazineethanesulfonic acid), TES buffer (2-{[tris(hydroxymethyl)methyl]amino}ethanesulfonic acid), MOPS buffer (3-(N-morpholino)propanesulfonic acid), PIPES buffer (piperazine-N,N′-bis(2-ethanesulfonic acid)), Cacodylate buffer (dimethylarsinic acid), SSC buffer (saline sodium citrate), or MES buffer (2-(N-morpholino)ethanesulfonic acid). The coupling reaction can comprise one or more buffers. In one embodiment, the coupling reaction comprises phosphate buffered saline. The coupling reaction comprising phosphate buffered saline can have a pH of between about 6 and 8 (e.g., about 7.2). In one embodiment, the coupling reaction comprises MES buffer. The coupling reaction comprising MES buffer can have a pH of between about 4 and 6 (e.g., about 5).

The choice of pH and/or buffer for the coupling reaction between the phage and particle can affect the stability of the phage and/or phage particle complex. In one embodiment, the pH of the coupling reaction is selected based upon stability of the phage at the pH chosen. In one embodiment, a phage-particle complex that is coupled using a buffer has increased stability compared to a phage-particle complex produced without using the buffer. In one embodiment, a phage-particle complex that is coupled using a buffer has increased stability compared to the phage-particle complex coupled in a different buffer. In one embodiment, a phage-particle complex that is coupled at a pH has increased stability compared to the phage-particle complex produced at a different pH. Stability of a phage-particle complex can be measured by the ability of the phage-particle complex to detect an antibody. For example, a coupling reaction comprising a buffer and a pH can produce a phage-particle complex that can detect a biomarker about 1, 2, 3, 4, 5, 6, 7, 8, 9, 10, 11, or 12 months longer than a coupling reaction comprising a different buffer or a different pH.

The choice of pH and/or buffer for the coupling reaction between the phage and the particle can affect the success rate of the coupling in producing an antibody detecting complex. For example, the coupling reaction between the phage and particle can produce an antibody detecting complex between 0 and 100% of the time. In one embodiment, the coupling reaction comprising a buffer and/or pH according to the methods disclosed herein produces an antibody detecting complex with a success rate that is higher than a coupling reaction without the buffer, using a different buffer, and/or using a different pH.

In another embodiment, a phage can be coupled to a particle via DNA double strand formation. A phage, probe, or particle can be covalently labeled with a single strand DNA in which a phage-particle complex is formed by complementary interactions of single strands. In one embodiment, a probe is labeled with 18-mer oligonucleotide and a phage is labeled with another 18-mer oligonucleotide in which the two oligonucleotides are complementary over a stretch of about 6, 8, 10, 12, or 16 nucleotides.

In one embodiment, the phage-particle complex comprises a linker. A linker can be used for coupling. In one embodiment, a linker is a chemical moiety that links, extends or conjugates two disparate structures. As described herein, a linker can comprise a variety of different structures and chemical compositions. The linker can also be used for a variety of different purposes and in a variety of different configurations.

In one embodiment, the linker moiety is coupled to the reactive group on the unnatural amino acid side chain in the polypeptide probe. In another embodiment, the linker can be associated with a reactive group on a phage or a particle. In another embodiment, a linker forms a bridge using covalent and/or non-covalent interactions between the polypeptide probe and the phage, for coupling the phage to a probe, or between the phage and the particle, for coupling the phage to the particle.

In one embodiment, a linker is used to attach the phage to the particle or probe via a reactive group on an unnatural amino acid side chain. In another embodiment, a linker is a chemical moiety that covalently joins the reactive group on the particle with the reactive group on the unnatural amino acid. Suitable linkers are known to those of skill in the art, and include those from any suitable class of compounds. Polymers or copolymers of organic acids, aldehydes, alcohols, thiols, amines, and the like, are examples of suitable linkers. For example, polymers or copolymers of hydroxy-, amino-, or di-carboxylic acids, such as glycolic acid, lactic acid, sebacic acid, or sarcosine can be used. Alternatively, one can use polymers or copolymers of saturated or unsaturated hydrocarbons such as ethylene glycol, propylene glycol, saccharides, and the like. The linker should be of an appropriate length that allows an attached polypeptide to interact freely with molecules in a sample.

In one embodiment, a linker is attached to the surface of a particle by a suitable functional group on the linker that reacts with a reactive group on the particle. For example, for a particle that has a hydroxyl group, one can form a siloxane bond by reacting the hydroxyl group with a trichlorosilyl or trisalkoxy group of a linker. Other suitable linkages, and functional groups that can be reacted to form a linker include, but are not limited to, thioether (reaction of thiol with maleimide or acrylamide), disulfide (activated disulfide with thiol), hydrazone (aldehyde or ketone with hydrazine or hydrazide), semicarbazone (aldehyde or ketone with semicarbazide), oxime (aldehyde or ketone with aminooxyacetyl), thiosemicarbazone (aldehyde or ketone with thiosemicarbazide), and thiazolidine (aldehyde and cystein). The linker can also be attached noncovalently to the particle. For example, one binding partner can be conjugated to a biotin moiety, which can form a strong noncovalent linkage to a conjugation partner that displays avidin. In one embodiment, the particle can be conjugated to biotin, and the phage to avidin. In another embodiment, the particle is conjugated to avidin and the phage to biotin. In yet another embodiment, a probe can be conjugated to biotin and the phage to avidin. In yet another embodiment, a probe is conjugated to avidin and the phage to biotin.

Chemical coupling can be achieved by a polysaccharide with a linker. For example, a coupling can be achieved by attaching one end of the polysaccharide linker to a probe and the other end to a phage polypeptide. Examples of linker molecules include, but not limited to, adipic acid dihydrazide, diaminohexane, amino epsilon caproic acid, and N-hydroxysuccinimide acid anhydride based heterobifunctional linkers such as N-succinimidyl 3-(2-pyridyldithio)priopionate (SPDP).

A wide variety of reactive groups are known. Reactive groups include, but are not limited to, amino, hydroxyl, carboxyl, carboxylate, aldehyde, ester, ether (e.g. thio-ether), amide, amine, nitrile, vinyl, sulfide, sulfonyl, phosphoryl, maleimide, N hydroxysuccinimide, sulfo-N-hydroxysuccinimide, nitrilotriacetic acid, activated hydroxyl, haloacetyl (e.g., bromoacetyl, iodoacetyl), activated carboxyl, hydrazide, epoxy, aziridine, sulfonylchloride, trifluoromethyldiaziridine, pyridyldisulfide, N-acyl-imidazole, imidazolecarbamate, vinylsulfone, succinimidylcarbonate, arylazide, anhydride, diazoacetate, benzophenone, isothiocyanate, isocyanate, imidoester, and fluorobenzene. In one embodiment, the phage is coupled to a carboxylated particle, such as a carboxylated microsphere. The phage can be coupled to the particle thorough carboxyl coupling, such as through carbodiimide coupling.

The coupling between the phage and the particle can incorporate a linker in various configurations. For example, the linker can be integral to the reactive group attached to the phage, integral to the reactive group attached to the particle, or two separate linkers can exist in the system where one is linked to the unnatural amino acid reactive group and the other is coupled to the particle reactive group. The linker can be reacted with either the phage or the particle prior to reaction with the other. For example, in the case where the linker forms part of the particle, the phage can be reacted with the reactive group on the linker before or after the linker is attached to the particle. Alternatively, the linker can be independent of the reactive groups on the phage and particle and reacts with those reactive groups to form a linker bridge between the phage and particle. Linkers can also serve as spacers where the incorporation of a spacer is desirable in order to add rotational freedom and reduce steric limitations on the chemical moieties used in the attachments.

In one embodiment, a probe linked to a particle via a phage is more stable in comparison to a probe linked to a particle not via a phage. In one embodiment, a probe linked to a particle via a phage is more stable in comparison to a directly probe linked to a particle. In one embodiment, the more stable probe is linked to the particle via a phage that is covalently linked to the particle. In yet another embodiment, the more stable probe is linked to the particle via a phage that is not covalently linked to the particle. The probe linked to the particle via a phage may be stable for more than 1, 2, 3, 4, 5, 6, 7, 8, 9, 10, 11, or 12 months. Stability of the probe can be measured by its ability to detect one or more known biomarkers.

A Panel of Phage-Particle Complexes

Also provided herein is a profiling panel comprising a plurality of phage-particle complexes. In one embodiment, the plurality of phage particle complexes comprises a plurality of probes, whereby the presence of a plurality of biological molecules can be analyzed. A panel of phage-particle complexes allows for the simultaneous analysis of a plurality of biological molecules to a plurality of polypeptide probes. A subset of the plurality of phage-particle complexes can comprise a probe that differs from probes of other phage-particle complexes present in the panel.

Different probes can be distinguished based on differing parameters or features of a particle. For example, particles with the same probe may be of the same size. Alternatively, particles can vary in size so that their size serves as a distinguishing parameter or unique sorting characteristic. In one embodiment, particles with different probes are about the same size and can be distinguished based on another parameter, such as a unique spectral property, which may be detected by a flow cytometer.

A panel can comprise a plurality of probes correlated with a disease or condition such as cancer. The probes can be used to detect one or more biological molecules correlated or associated with a cancerous tissue, metastatic cancer, localized cancer that is likely to metastasize, pre-cancerous tissue that is likely to become cancerous, and pre-cancerous tissue not likely to become cancerous. In one embodiment, a panel of phage-particle complexes may be analyzed alone or in combination with other sets in order to detect a condition or disease.

In one embodiment, the panel is an antibody profiling panel comprising a plurality of antibody detecting complexes, wherein each of the antibody detecting complex comprises a polypeptide probe. The probe is capable of being specifically bound by an antibody and is present on a phage, and the phage is coupled to a particle. The profiling panel can be used to analyze the presence of a plurality of antibodies, such as autoantibodies, against a plurality of polypeptide probes.

A profiling panel comprising a plurality of phage-particle complexes can comprise 2-100 probes, 50-200 probes, 100-500 probes 200-750 probes, 200-1000 probes, 2-5,000 probes or 2-10,000 probes. In one embodiment, a profiling panel comprises at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 probes. In another embodiment, an antibody profiling panel comprises at least about 50, 100, 150, 200, 250, 500, 750, 1000, 5000, 10,000, 15,000, 20,000, 25,000, 30,000, 40,000, 50,000, 60,000, 70,000, 75,000, or 100,000 probes. In one embodiment, the probes are polypeptide probes. In another embodiment, the probes are molecules that mimic an epitope bound by a particular antibody.

Each probe can be directed to detect one type of biological molecule, such as a multitude of probes directed to detect a specific antibody. Alternatively, each probe can be directed to detect different biological molecules, such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 biological molecules, such as different antibodies.

In one embodiment, a profiling panel is an antibody profiling panel comprising at least 2, 3, 4, 5, 6, 7, 8, 9, 10, 11, 12, 13, or 14 polypeptide probes, wherein the polypeptide probes are a fragment of a protein encoded by a gene, or a fragment encoded by a sequence of a UTR region of a gene, such as genes listed in Table 1. In one embodiment, an antibody profiling panel comprises a plurality of polypeptide probes, wherein at least a subset of the polypeptide probes is a fragment of a protein encoded by a gene, or a fragment encoded by a sequence of a UTR region of a gene, wherein the gene is AZGP1, DCHS1, FAM53B, MAPKKK9, Deaminase Domain, ARF6, CEP 164, ADAM9, SFRS14, Cox5a, TIMP2, BRMSL1, Nucleolin, NKX3-1, CSNK2A2, DSC3, RPL34, 3′ UTR Ropporin, AURKAIP1,5′-UTR BMI1, LAMR1, Contig GRCh37.p2 (1371451::1372008), PSA, RPSA (minus strand), RBM6 (minus strand), or hypothetical protein XP_{—}353238. In one embodiment, an antibody profiling panel comprises a plurality of polypeptide probes, wherein at least a subset of the polypeptide probes comprises SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 74, SEQ ID NO: 77, SEQ ID NO: 80, or SEQ ID NO: 82, or a fragment thereof. In another embodiment, an antibody profiling panel comprises a plurality of polypeptide probes, wherein at least a subset of the polypeptide probes comprises a polypeptide encoded by SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 75, SEQ ID NO: 78, SEQ ID NO: 81, OR SEQ ID NO: 83, or a fragment thereof. In another embodiment, an antibody profiling panel comprises a plurality of polypeptide probes, wherein at least a subset of the polypeptide probes is encoded by SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65 SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 76, or SEQ ID NO: 79, or a fragment thereof. Other suitable markers can include those known in the art, such as biomarkers disclosed in U.S. patent application Ser. No. 13/050,544 and U.S. Pat. No. 7,858,323, which are hereby incorporated by reference in their entirety.

In yet another embodiment, an antibody profiling panel comprises one or more polypeptide probes of the protein PSA, or fragment of PSA, in combination with one or more of the polypeptide probes discussed herein. In another embodiment, an antibody profiling panel can comprise polypeptide probes including a full-length protein or fragment of PSA and a full-length protein encoded by a gene, fragment of a protein encoded by a gene, or a fragment encoded by a sequence of a UTR region of a gene, wherein the gene is AZGP1, DCHS1, FAM53B, MAPKKK9, Deaminase Domain, ARF6, CEP 164, ADAM9, SFRS14, Cox5a, TIMP2, BRMSL1, Nucleolin, NKX3-1, CSNK2A2, DSC3, RPL34, 3′ UTR Ropporin, AURKAIP1,5′-UTR BMI1, LAMR1, Contig GRCh37.p2 (1371451::1372008), PSA, RPSA (minus strand), RBM6 (minus strand), or hypothetical protein XP_{—}353238. In another embodiment, an antibody profiling panel can comprise a plurality of polypeptide probes, wherein the probes includes a full-length protein or fragment of PSA and one or probes comprising a peptide sequence, or fragment thereof, as listed in Table 1. In one embodiment, an antibody profiling panel can comprise a plurality of polypeptide probes, wherein the probes includes a full-length protein or fragment of PSA and one or more probes comprising SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 74, SEQ ID NO: 77, SEQ ID NO: 80, or SEQ ID NO: 82, or a fragment thereof. In another embodiment, an antibody profiling panel can comprise a plurality of polypeptide probes, wherein the probes includes a full-length protein or fragment of PSA and one or more probes comprising a polypeptide encoded by SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 75, SEQ ID NO: 78, SEQ ID NO: 81, OR SEQ ID NO: 83, or a fragment thereof. In another embodiment, an antibody profiling panel can comprise a plurality of polypeptide probes, wherein the probes includes a full-length protein or fragment of PSA and one or more probes comprising a polypeptide encoded by SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65 SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 76, or SEQ ID NO: 79. Other suitable markers can include those known in the art, such as biomarkers disclosed in U.S. patent application Ser. No. 13/050,544, U.S. patent application Ser. No. 13/072,542, and U.S. Pat. No. 7,858,323, which are hereby incorporated by reference in their entirety.

In one embodiment, a PSA polypeptide probe can be combined with any two or more of the polypeptide probes described herein, such as a polypeptide probe derived from a protein encoded by a gene, fragment of a protein encoded by a gene, or a fragment encoded by a sequence of a UTR region of a gene, wherein the gene is AZGP1, DCHS1, FAM53B, MAPKKK9, Deaminase Domain, ARF6, CEP 164, ADAMS, SFRS14, Cox5a, TIMP2, BRMSL1, Nucleolin, NKX3-1, CSNK2A2, DSC3, RPL34, 3′ UTR Ropporin, AURKAIP1,5′-UTR BMI1, LAMR1, Contig GRCh37.p2 (1371451::1372008), PSA, RPSA (minus strand), RBM6 (minus strand), or hypothetical protein XP_{—}353238. In another embodiment, a PSA polypeptide probe can be combined with at least 3, 4, 5, 6, 7, 8, 9, 10, 11, 12, 13 or 14 of polypeptide probes disclosed herein, such as listed in Table 1. In one embodiment, a PSA polypeptide probe can be combined with a polypeptide probe comprising SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 74, SEQ ID NO: 77, SEQ ID NO: 80, or SEQ ID NO: 82, or a fragment thereof. In another embodiment a PSA polypeptide probe can be combined with a polypeptide probe comprising a polypeptide encoded by SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 75, SEQ ID NO: 78, SEQ ID NO: 81, OR SEQ ID NO: 83, or a fragment thereof. In another embodiment, a polypeptide probe comprises a polypeptide encoded by SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65 SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 76, or SEQ ID NO: 79, or a fragment thereof. Other suitable markers for combination with a PSA polypeptide probe were disclosed in U.S. patent application Ser. No. 13/050,544, U.S. patent application Ser. No. 13/072,542, and U.S. Pat. No. 7,858,323, which are hereby incorporated by reference in their entirety.

Detecting a Disease or Condition

A disease or condition can be detected with one or more methods and compositions disclosed herein. For example, detection of a biomarker using one or more probes present in a phage-particle complex or panel of complexes can be used to detect a condition or disease, such as provide a diagnosis, prognosis, or theranosis of a condition or disease. In one embodiment, detection of biomarker in a sample is used to diagnosis or determine the likelihood of a disease or condition. In another embodiment, a phage-particle complex or panel of complexes is used to screen for a condition or disease. In yet another embodiment, a phage-particle complex or panel of complexes can be used to determine a specific stage or sub-type of a disease or condition.

In one embodiment, the information obtained by using a phage-particle complex or panel of complexes is used to determine a prognosis, such as the outcome or predicted outcome of a disease or condition. In yet another embodiment, the information obtained by using a phage-particle complex or panel of complexes is used to determine a theranosis, wherein an appropriate course of treatment is selected or determined. The information obtained from a method disclosed herein can thus provide for the personalization of diagnosis and treatment.

In one embodiment, a disease or condition is detected with increased accuracy, such as with increased specificity or sensitivity. The sensitivity can be determined by: (number of true positives)/(number of true positives+number of false negatives), whereas the specificity can be determined by: (number of true negatives)/(number of true negatives+number of false positives).

In one embodiment, the cancer can be detected (e.g. prognosed, theranosed, etc.) with at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sensitivity. In another embodiment, the cancer can be detected (e.g. prognosed, theranosed, etc.) with at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% specificity.

In one embodiment, specificity of detection can be altered by altering the probe make-up of a set. For example, sensitivity of a diagnostic and/or prognostic assay (e.g., autoantibody detection assay) can be increased by increasing the number of probes, increasing the diversity of probes (e.g, utilizing probes comprising distinct epitopes from the same and/or different markers), or tailoring the probes to a particular subject or cancer to be diagnosed/prognosed. Furthermore, the confidence level for determining the specificity, sensitivity, or both, may be with at least 90, 91, 92, 93, 94, 95, 96, 97, 98, or 99% confidence.

Information or data from a binding assay using one or more phage-particle complexes disclosed herein can be prepared in a format suitable for interpretation by a treating clinician. In one embodiment, rather than providing raw expression data, the prepared format represents a diagnosis, screening or risk assessment (e.g., likelihood of metastasis or PSA failure or the development of high prostate specific antigen levels in a patient following prostate cancer therapy (e.g., surgery)) for the subject, along with recommendations for particular treatment options. The data can be displayed to the clinician by any suitable method. In one embodiment, the profiling service generates a report that is printed for the clinician (e.g., at the point of care). In another embodiment, the report is displayed to the clinician on a computer monitor.

In one embodiment, the information is first analyzed at the point of care or at a regional facility. The raw data is then sent to a central processing facility for further analysis. In one embodiment, further analysis comprises converting the raw data to information useful for a clinician or subject, such as a patient. The central processing facility can provide the advantage of privacy (all data is stored in a central facility with uniform security protocols), speed, and uniformity of data analysis. The central processing facility can also control the fate of the data following treatment of a subject. In one embodiment, using an electronic communication system, the central facility provides data to the clinician, the subject, researchers, or any other individual. In one embodiment, a subject is able to directly access the data using the electronic communication system. In another embodiment, a subject chooses further intervention or counseling based on the result. In one embodiment, the data is used for research use. The data can be used to further optimize the inclusion or elimination of markers as useful indicators of a particular condition or stage of disease.

Cancer

One or more phage-particle complexes disclosed herein can be used to detect a cancer. In one embodiment, the cancer is an epithelial cancer. In yet another embodiment, the cancer is prostate cancer. In yet another embodiment, the cancer is lung cancer. In yet another embodiment, the cancer is breast cancer.

One or more phage-particle complexes disclosed herein can be used to characterize a cancer such as, but not limited to, a carcinoma, a sarcoma, a lymphoma, a germ cell tumor, or a blastoma. A carcinoma includes, but is not limited to, epithelial neoplasm, squamous cell neoplasm, squamous cell carcinoma, basal cell neoplasm, basal cell carcinoma, transitional cell papilloma and carcinoma, adenoma, adenocarcinoma, linitis plastica insulinoma, glucagonoma, gastrinoma, vipoma, cholangiocarcinoma, hepatocellular carcinoma, adenoid cystic carcinoma, carcinoid tumor of appendix, prolactinoma, oncocytoma, hurthle cell adenoma, renal cell carcinoma, grawitz tumor, multiple endocrine adenoma, endometrioid adenoma, adnexal and skin appendage neoplasms, mucoepidermoid neoplasms, cystic, mucinous and serous neoplasm, cystadenoma, pseudomyxoma peritonei, ductal, lobular and medullary neoplasms, acinar cell neoplasms, complex epithelial neoplasms, warthin's tumor, thymoma, specialized gonadal neoplasms, sex cord stromal tumor, thecoma, granulosa cell tumor, arrhenoblastoma, sertoli leydig cell tumor, glomus tumors, paraganglioma, pheochromocytoma, glomus tumor, nevi and melanomas, melanocytic nevus, malignant melanoma, melanoma, nodular melanoma, dysplastic nevus, lentigo maligna melanoma, superficial spreading melanoma, and malignant acral lentiginous melanoma.

A sarcoma includes, but is not limited to, Askin's tumor, chondrosarcoma, Ewing's sarcoma, malignant schwannoma, osteosarcoma, soft tissue sarcomas including: alveolar soft part sarcoma, angiosarcoma, cystosarcoma phyllodes, dermatofibrosarcoma, desmoid tumor, desmoplastic small round cell tumor, epithelioid sarcoma, extraskeletal chondrosarcoma, extraskeletal osteosarcoma, fibrosarcoma, hemangiopericytoma, hemangiosarcoma, kaposi's sarcoma, leiomyosarcoma, liposarcoma, lymphangiosarcoma, lymphosarcoma, malignant fibrous histiocytoma, neurofibrosarcoma, rhabdomyosarcoma, and synovialsarcoma.

A lymphoma includes, but is not limited to, chronic lymphocytic leukemia/small lymphocytic lymphoma, B-cell prolymphocytic leukemia, lymphoplasmacytic lymphoma (such as Waldenström macroglobulinemia), splenic marginal zone lymphoma, plasma cell myeloma, plasmacytoma, monoclonal immunoglobulin deposition diseases, heavy chain diseases, extranodal marginal zone B cell lymphoma, also called malt lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt lymphoma/leukemia, T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, anaplastic large cell lymphoma, classical hodgkin lymphomas (nodular sclerosis, mixed cellularity, lymphocyte-rich, lymphocyte depleted or not depleted), and nodular lymphocyte-predominant Hodgkin lymphoma.

A germ cell tumor includes, but is not limited to, germinoma, dysgerminoma, seminoma, nongerminomatous germ cell tumor, embryonal carcinoma, endodermal sinus turmor, choriocarcinoma, teratoma, polyembryoma, and gonadoblastoma. A blastoma includes, but is not limited to, nephroblastoma, medulloblastoma, and retinoblastoma. Other cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma.

In one embodiment, presence of an immune response to a specific protein expressed in cancerous cells can be indicative of a presence of cancer. Accordingly, provided herein is a method (e.g., diagnostic or screening method) for detecting a presence of an antibody, such as an autoantibody, to a tumor or tumor-associated antigen. In one embodiment, the presence of an antibody in cancerous but not cancerous cells is indicative of the presence of cancer. In one embodiment, the antibody is an antibody to a tumor antigen.

A cancer can be detected by determining the presence, absence, or level of one or more autoantibodies in a sample. The level, presence, or absence of an autoantibody can be determined by detecting the binding of one or more autoantibodies to a polypeptide probe. An autoantibody refers to an antibody produced by a host (with or without immunization) and directed to a host antigen (such as a tumor antigen). Tumor-associated antigens recognized by humoral effectors of the immune system are an attractive target for diagnostic and therapeutic approaches to human cancer.

The presence of an immune response to specific proteins expressed in cancerous cells can be indicative of the presence of cancer. Accordingly, provided herein are methods (e.g., diagnostic methods) for detecting the presence of autoantibodies to tumor and/or tumor-associated antigens. For example, where the presence of an autoantibody in cancerous but not cancerous cells is indicative of the presence of cancer, autoantibodies to the tumor antigens are detected.

For example, the presence of an autoantibody to a specific protein may be indicative of a cancer. In addition, certain autoantibodies may be indicative of a specific stage or sub-type of the same cancer. The information obtained by detecting autoantibodies as described herein can be used to determine prognosis and appropriate course of treatment. For example, it is contemplated that individuals with a specific autoantibody or stage of cancer can respond differently to a given treatment than individuals lacking the antibody. The information obtained from the diagnostic methods of the present invention thus provides for the personalization of diagnosis and treatment.

Depending on the results, a cancer (or absence of cancer) can be detected. A method disclosed herein can comprise detecting a plurality of antibodies, such as through the detection of binding of one or more antibodies that bind to a plurality of polypeptide probes. In one embodiment, the antibodies are autoantibodies. In another embodiment, the antibodies are antibodies to foreign antigens. In one embodiment, the method comprises detecting in a sample one or more antibodies that binds to a panel of polypeptide probes, wherein the panel comprises 2-100 probes, 50-200 probes, 100-500 probes 200-750 probes, 200-1000 probes, 2-5,000 probes or 2-10,000 probes. In another embodiment, the panel of polypeptide probes comprises at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 polypeptide probes. In another embodiment, the panel comprises at least about 50, 100, 150, 200, 250, 500, 750, 1000, 5000, 10,000, 15,000, 20,000, 25,000, 30,000, 40,000, 50,000, 60,000, 70,000, 75,000, or 100,000 polypeptide probes. In one embodiment, the panel comprises a plurality of polypeptide probes, wherein a subset of the probes comprise fragments of the same full-length protein, such that autoantibodies to different epitopes bind to the different probes and indicate a presence of an immune response, or antibody, to the full-length protein.

Any of the proteins listed in Table 1, or proteins encoded by the genes listed in Table 1, in any combination, can be utilized to detect a presence of an antibody, such as an autoantibody, in a subject. In one embodiment, detection of an autoantibody to a protein encoded by a gene, a fragment encoded by a sequence of a UTR region of a gene, or fragment of a protein encoded by a gene, wherein the gene is AZGP1, DCHS1, FAM53B, MAPKKK9, Deaminase Domain, ARF6, CEP 164, ADAMS, SFRS14, Cox5a, TIMP2, BRMSL1, Nucleolin, NKX3-1, CSNK2A2, DSC3, RPL34, 3′ UTR Ropporin, AURKAIP1,5′-UTR BMI1, LAMR1, Contig GRCh37.p2 (1371451::1372008), PSA, RPSA (minus strand), RBM6 (minus strand), or hypothetical protein XP_{—}353238, or any combination thereof, is indicative of a presence of prostate cancer in a subject. In another embodiment, any combination of two or more proteins (e.g., cancer markers) or fragments thereof is used to detect one or more autoantibodies (e.g., a panel consisting of one or more full-length or fragments of the polypeptides listed in Table 1).

In one embodiment, the method comprises detecting one or more antibodies that bind to at least 8, 9, 10, 11, 12, 13, or 14 polypeptide probes, wherein the polypeptide probes are full-length or fragments of proteins encoded by the genes listed in Table 1, or polypeptides encoded by the UTR sequence of the gene. In one embodiment, the method comprises detecting one or more antibodies that bind to at least 1, 2, 3, 4, 5, 6, 7, 8, 9, 10, 11, 12, 13, or 14 polypeptide probes, wherein the polypeptide probes are full-length or fragments of proteins encoded by the genes listed in Table 1, or polypeptides encoded by the UTR sequence of the gene. In one embodiment, the antibody profiling panel comprises a plurality of polypeptide probes, wherein one or more polypeptide probes is a protein or fragment of a protein encoded by AZGP1, DCHS1, FAM53B, MAPKKK9, Deaminase Domain, ARF6, CEP 164, ADAM9, SFRS14, Cox5a, TIMP2, BRMSL1, Nucleolin, NKX3-1, CSNK2A2, DSC3, RPL34, 3′ UTR Ropporin, AURKAIP1,5′-UTR BMI1, LAMR1, Contig GRCh37.p2 (1371451::1372008), PSA, RPSA (minus strand), RBM6 (minus strand), or hypothetical protein XP_{—}353238, or any combination thereof. Other suitable markers can include those known in the art, such as biomarkers disclosed in U.S. patent application Ser. No. 13/050,544, U.S. patent application Ser. No. 13/072,542, and U.S. Pat. No. 7,858,323, which are hereby incorporated by reference in their entirety.

The level, presence or absence of an autoantibody can be determined by detecting the binding of one or more autoantibodies to a polypeptide probe. Detection of the antibody can be either quantitative or qualitative. For quantitative assays, the amount of autoantibody detected can be compared to a control or reference to determine whether an autoantibody is overexpressed or underexpressed in a sample. For example, the control or reference can be a normal sample or a sample from a known disease state, such as a cancer sample.

The detection of one or more antibodies from a sample, such as described herein, can be used in conjunction with one or more other tests used for detecting or screening for cancer. The antibody detection can be used prior to, concurrent with, or subsequent to one or more other tests. In one embodiment, a genetic test for a mutation or expression level of one or more genes can be used in conjunction with determining the antibody profile of a subject.

Antibody detection can provide a non-invasive, inexpensive means for detecting or screening for a cancer. Thus, in one embodiment, the detection of a level, presence or absence of one or more antibodies using an antibody detecting complex as described herein, can be used to determine whether a second sample or additional analysis of a sample from a subject is to be performed. In one embodiment, after detecting an expression level of one or more antibodies of sample obtained from subject to one or more polypeptide probes comprising a fragment of a protein encoded by, or a polypeptide encoded by a UTR sequence of AZGP1, DCHS1, FAM53B, MAPKKK9, Deaminase Domain, ARF6, CEP 164, ADAM9, SFRS14, Cox5a, TIMP2, BRMSL1, Nucleolin, NKX3-1, CSNK2A2, DSC3, RPL34, 3′ UTR Ropporin, AURKAIP1,5′-UTR BMI1, LAMR1, Contig GRCh37.p2 (1371451::1372008), PSA, RPSA (minus strand), RBM6 (minus strand), or hypothetical protein XP_{—}353238, a biopsy can be recommended for the subject. Other suitable markers can include those known in the art, such as biomarkers disclosed in U.S. patent application Ser. No. 13/050,544, U.S. patent application Ser. No. 13/072,542, and U.S. Pat. No. 7,858,323, which are hereby incorporated by reference in their entirety.

In another embodiment, an expression level for one or more antibodies from a subject can be detected, and based on the expression level of the one or more antibodies, the subject can be identified as suspected of having cancer. In one embodiment, the subject is detected as having a high probability or likelihood of having cancer. Based on the detection or expression level of the one or more antibodies, a recommendation that a biopsy be obtained can be made for the subject. In another embodiment, if there is a lack of detection or expression of the one or more antibodies, further analysis is not recommended and a biopsy not be obtained.

In another embodiment, prior to detecting one or more antibodies from a subject, the subject is suspected of having cancer. The subject can have had a genetic test for a mutation or gene expression analysis, image analysis (such as magnetic resonance imaging (MRI), positron emission tomography (PET) scan, computerized tomography (CT) scan, nuclear magnetic resonance (NMR)), or biopsy, and have inconclusive or uncertain results. Thus, prior to further analysis and treatment for a suspected cancer, the subject can seek further verification of their likelihood of having a cancer, or their diagnosis, prognosis, or theranosis of a cancer.

In one embodiment, an antibody profiling panel described herein can be used in conjunction with a separate test which determines a presence or level of PSA (e.g., a serum PSA test). In one embodiment, the panels is utilized to diagnose or prognose a presence of a cancer (e.g., prostate cancer) in a subject. In one embodiment, a subject is suspected of having prostate cancer based on their PSA level, age, or both. A subject can be male and over 30, 35, 40, 45, 50, 55, 60, 65, 70 or 75 years of age. In another embodiment, the subject is between 30-80, 40-75, 45-75, or 50-75 years of age. In another embodiment, the subject had a PSA blood test, digital rectal exam, or both. In yet another embodiment, the subject may have a PSA level of at least about 1.0, 1.5, 2.0, 2.5, or 4.0 ng/ml. The subject can have a PSA level of between about 1.0-15 ng/ml, 2.0-15 ng/ml, or 2.5-10 ng/ml.

In one embodiment, a biological sample from a subject, such as a subject with a PSA level greater than about 2.5 ng/ml, is contacted with one or more probes for an antibody, such as one or more probes for an autoantibody. Based on the expression level of the antibody, a biopsy for the subject can be recommended. The antibody test can comprise detecting one or more antibodies in a sample that bind to a polypeptide probe as described herein. In another embodiment, the antibody test is an autoantibody test.

In one embodiment, the antibody binds a polypeptide probe comprising a full-length or fragment of a protein encoded by, or a polypeptide encoded by a UTR of, AZGP1, DCHS1, FAM53B, MAPKKK9, Deaminase Domain, ARF6, CEP 164, ADAMS, SFRS14, Cox5a, TIMP2, BRMSL1, Nucleolin, NKX3-1, CSNK2A2, DSC3, RPL34, 3′ UTR Ropporin, AURKAIP1,5′-UTR BMI1, LAMR1, Contig GRCh37.p2 (1371451::1372008), PSA, RPSA (minus strand), RBM6 (minus strand), or hypothetical protein XP_{—}353238. Other suitable markers can include those known in the art, such as biomarkers disclosed in U.S. patent application Ser. No. 13/050,544, U.S. patent application Ser. No. 13/072,542, and U.S. Pat. No. 7,858,323, which are hereby incorporated by reference in their entirety. In one embodiment, a polypeptide probe comprises SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 74, SEQ ID NO: 77, SEQ ID NO: 80, or SEQ ID NO: 82, or a fragment thereof. In another embodiment, a polypeptide probe comprises a polypeptide encoded by SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 75, SEQ ID NO: 78, SEQ ID NO: 81, OR SEQ ID NO: 83, or a fragment thereof. In another embodiment, a polypeptide probe comprises a polypeptide encoded by SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65 SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 76, or SEQ ID NO: 79, or a fragment thereof.

If a biopsy is recommended and the biopsy is positive for a cancer such as prostate cancer, a biological sample obtained from the subject can be contacted with one or more probes for an antibody, which can be the same or different, as those used in deciding whether to obtain a biopsy. Based on the expression level of antibodies in the sample, a prognosis for the cancer can be provided.

Thus, in one embodiment, a method of detecting a cancer from a subject with a positive biopsy result is provided. In another embodiment, the subject has not yet provided a sample for detecting one or more antibodies. In yet another embodiment, the subject has provided an initial sample for detecting one or more antibodies and detection of the one or more antibodies is used in deciding whether a biopsy is obtained. Furthermore, in one embodiment, detection of one or more antibodies is used for a diagnosis, prognosis or theranosis of a cancer, such as prostate cancer. In one embodiment, a cancer is classified based on the detection of one or more antibodies to one or more polypeptide probes disclosed herein. In one embodiment, the cancer is classified as aggressive or malignant. In another embodiment, the cancer is classified as indolent or benign. Furthermore, after classification, detection of one or more antibodies from a sample from the subject can be used to select a treatment or therapeutic for the cancer.

Other Diseases or Conditions

A phage-particle complex can also be used to detect a cardiovascular disease or condition. For example, an antibody detecting complex can be used to detect circulating autoantibodies against cardiovascular membrane receptors or other proteins correlated with cardiovascular disease. For example, circulating autoantibodies against apolipoprotein A1 or C-reactive protein has been described (O'Neill et al, Arthritis Rheum, Jan. 7, 2010, Epub ahead of print). Autoantibodies against Apo B-100 in carotid stenosis and other cardiovascular events has also been described (Fredrikson et al., Atherosclerosis, 194:e188-92, 2007).

A cardiovascular disease or disorder that can be detected using one or more compositions and methods disclosed herein includes, but is not limited to, atherosclerosis, congestive heart failure, vulnerable plaque, stroke, ischemia, chronic rheumatic heart disease, hypertensive disease, ischemic heart disease, pulmonary circulatory disease, heart disease, cerebrovascular disease, diseases of arteries, arterioles and capillaries and diseases of veins and lymphatics. The phenotype can also be a cardiovascular disease, such as. The cardiovascular disease or condition can be high blood pressure, stenosis, vessel occlusion or a thrombotic event.

A phage-particle complex or panel of complexes can also be used to detect an infectious disease or condition. The infectious disease can be a bacterial, viral or yeast infection. For example, the disease or condition may be Whipple's Disease, Prion Disease, cirrhosis, methicillin-resistant staphylococcus aureus, HIV, hepatitis, syphilis, meningitis, malaria, tuberculosis, or influenza. For example, an antibody detecting complex can be used to detect an antibody in a sample from a subject. In one embodiment, an antibody detecting complex can comprise a probe derived from a polypeptide encoded or produced by a pathogen, such as a virus, bacteria, or fungus. In one embodiment, a probe can be derived from a viral protein, such as from an influenza protein, HIV or HCV-like particle.

A phage-particle complex can also be used to detect an inflammatory condition or disease, immune disease, or autoimmune disease. For example, the disease may be inflammatory bowel disease (IBD), Crohn's disease (CD), ulcerative colitis (UC), pelvic inflammation, vasculitis, psoriasis, diabetes, autoimmune hepatitis, Multiple Sclerosis, Myasthenia Gravis, Type I diabetes, Rheumatoid Arthritis, Psoriasis, Systemic Lupus Erythematosis (SLE), Hashimoto's Thyroiditis, Grave's disease, Ankylosing Spondylitis Sjogrens Disease, CREST syndrome, Scleroderma, Rheumatic Disease, organ rejection, Primary Sclerosing Cholangitis, or sepsis.

A phage-particle complex or panel of complexes can also be used to detect an autoimmune disease or condition. For example, an antibody detecting complex can be used to detect an antibody in a sample from a subject and used to detecting an autoimmune disease for the subject. For example, anti-nuclear antibodies are present in higher than normal concentration in autoimmune diseases (Bogdanos et al., Semin. Liver Dis. 29:241-53, 2009). Other autoantibodies associated with autoimmune diseases that have been identified include anti-actin antibodies, anti-ganglioside antibodies such as anti-GD3 antibody (Guillain-Barré syndrome), anti-GM1 antibody (travelers diarrhea) or anti-GQ1b antibody (Miller-Fisher syndrome, anti-gastric parietal cell antibody, anti-glomerular basement membrane antibody (anti-GBM antibody), anti-Hu antibody, anti-Jo 1 antibody, anti-liver/kidney microsomal 1 antibody (anti-LKM 1 antibodies), anti-Ku antibody, anti-mitochondrial antibodies such as anti-pyruvate dehydrogenase antibody, anti-2-oxo-glutarate dehydrogenase antibody or anti-branched chain 2-oxo-acid dehydrogenase antibody, anti-neutrophil cytoplasmic antibody (ANCA), anti-nuclear antibodies (ANA) such as anti-p62 antibodies in primary biliary cirrhosis, anti-sp100 antibodies in primary biliary cirrhosis, anti-glycoprotein210 antibodies in primary biliary cirrhosis, anti-ds DNA antibody, or anti-extractable nuclear antigen antibodies (anti-Ro antibody, anti-La antibody), anti-PM/Scl (anti-exosome) antibody, anti-Scl 70 antibody (in sclerosis and scleroderma) such as anti-topoisomerase antibody, anti-centromere antibody, anti-smooth muscle antibody, anti-transglutaminase antibodies such as anti-tTG antibody or anti-eTG antibody (dermatitis herpetiformis), Rheumatoid factor (RF), and Lupus anticoagulant (i.e., Lupus antibody) such as anti-thrombin antibodies.

Autoimmune conditions or diseases can include, but not be limited to, lupus (such as, but not limited to, systemic lupus erythematosus (SLE), discoid lupus, and lupus nephritis), sarcoidosis, inflammatory arthritis (such as, but not limited to, juvenile arthritis, rheumatoid arthritis, and psoriatic arthritis), Multiple Ssclerosis, Crohn's disease, Celiac's disease (such as gluten-sensitive enteropathy), diabetes, psoriasis, scleroderma, myasthenia gravis, Grave's disease, Hasimoto's thyroiditis, chronic fatigue immune dysfunction syndrome (CFIDS), pulmonary interstitial fibrosis, asthma, IgE-mediated allergy, atherosclerosis, Alzheimer's disease, Sjögren's syndrome, and ulcerative colitis.

A phage-particle complex or panel of complexes can also be used to detect a neurological disease or condition. For example, autoantibodies against beta-amyloid peptide have been found in the serum (Sohn et al., Front. Biosci., 14:3879, 2009). Roche et al. reported a method of profiling of autoantibodies in cerebrospinal fluid using a microarray platform (J. Immunol. Methods, 338:75-78, 2008). Autoantibodies associated with multiple sclerosis have been reported as well (Somers et al., J. Immunol., 180:3957-63, 2008). An antibody profiling complex or panel can be used to detect one or more autoantibodies in a sample of a subject and a neurological disorder detected.

The neurological disease or condition can be, but not limited to, Multiple Sclerosis (MS), Parkinson's Disease (PD), Alzheimer's Disease (AD), schizophrenia, bipolar disorder, depression, autism, Prion Disease, Pick's disease, dementia, Huntington disease (HD), Down's syndrome, cerebrovascular disease, Rasmussen's encephalitis, viral meningitis, neurospsychiatric systemic lupus erythematosus (NPSLE), amyotrophic lateral sclerosis, Creutzfeldt-Jacob disease, Gerstmann-Straussler-Scheinker disease, transmissible spongiform encephalopathy, ischemic reperfusion damage (e.g. stroke), brain trauma, microbial infection, chronic fatigue syndrome, inflammatory diseases of the central nervous system, hereditary and degenerative diseases of the central nervous system, pain, headache syndromes, disorders of the central nervous system, and disorders of the peripheral nervous system. The neurological disease or condition can be fibromyalgia, chronic neuropathic pain, or peripheral neuropathic pain.

The present disclosure is not limited to the embodiments described herein, but is capable of modification within the scope of the appended claims. Those skilled in the art will recognize, or be able to ascertain using no more than routine experimentation, many equivalents of the specific embodiments of the present disclosure described herein.

### EXAMPLES

### Example 1

Bacterophage were coupled to Luminex® microspheres. Bacteriophage were density-gradient purified and dialyzed against 1× phosphate buffered saline (PBS). About 40 μg of purified phage was coupled using carboxyl coupling chemistry at a pH of 7.2. Stock uncoupled microspheres were resuspended according to the instructions described in the Product Information Sheet provided by Luminex®. About 5.0×10⁶ of the stock microspheres were transferred to a microcentrifuge tube and pelleted by microcentrifugation at ≧8000×g for 1-2 minutes.

The supernatant was removed and the pelleted microspheres were resuspended in 100 μL, dH2O through a combination of vortexing and sonication for approximately 20 seconds. The microspheres were pelleted by microcentrifugation at ≧8000×g for 1-2 minutes. The supernatant was removed and the washed microspheres were resuspended in 80 μL 100 mM Monobasic Sodium Phosphate, pH 6.2 through a combination of vortexing and sonication for approximately 20 seconds. To the microsphere suspension, 10 μL of 50 mg/mL N-hydroxysulfosuccinimide (Sulfo-NHS) diluted in dH₂O was added followed by gentle vortexing. To this suspension, 10 μL of 50 mg/mL 1-Ethyl-3-(3-dimethylaminopropyl)carbodiimide (EDC) diluted in dH₂O was added and mixed by gentle vortexing.

The microsphere suspension mixture was incubated for 20 minutes at room temperature with gentle mixing by vortex at 10 minute intervals. The activated microspheres were pelleted by microcentrifugation at ≧8000×g for 1-2 minutes. The supernatant was removed and the microspheres were resuspended in 250 μL of 50 mM 2-(N-morpholino)ethanesulfonic acid (MES), pH 5.0 by a combination of vortexing and sonication for approximately 20 seconds. Coupling was performed in 100 mM MES, pH 6.0. Following coupling, microspheres were pelleted by microcentrifugation at ≧8000×g for 1-2 minutes and the supernatant removed. The microspheres were washed two times with 50 mM MES, pH 5.0. Each wash incorporated the steps of resuspension in 50 mM MES, pH 5.0 through vortexing and sonication for 20 seconds, pelleting by microcentrifugation at ≧8000×g for 1-2 minutes, and removal of the supernatant.

The activated and washed microspheres were resuspended in 100 μL of 50 mM MES, pH 5.0 by vortex and sonication for approximately 20 seconds. An amount of phage (125, 25, 5 or 1 μg) was added to the resuspended microspheres and the total volume was brought to 900 μL with 1×PBS pH 7.2. This coupling reaction was mixed by vortex and incubated for 2 hours at room temperature with mixing by rotation. The coupled microspheres were pelleted by microcentrifugation at ≧8000×g for 1-2 minutes. The supernatant was removed and the pelleted microspheres were resuspended in 500 μL of PBS-TBN by vortex and sonication for approximately 20 seconds. Either PBS-TBN (PBS, 0.1% BSA, 0.02% Tween-20, 0.05% Azide, pH 7.4) or PBS-BN (PBS, 1% BSA, 0.05% Azide, pH 7.4) was used as Blocking/Storage Buffer. The mix was further incubated for 30 minutes with mixing (by rotation) at room temperature. The coupled microspheres were pelleted by microcentrifugation at ≧8000×g for 1-2 minutes. The supernatant was removed and the microspheres were washed 2 times using either PBS-TBN or PBS, 0.05% Tween-20. Each wash incorporated the steps of resuspension in 1 mL of buffer through vortexing and sonication for 20 seconds, pelleting by microcentrifugation at ≧8000×g for 1-2 minutes, and removal of the supernatant. The coupled and washed microspheres were resuspended in 250-1000 μL of PBS-TBN and counted by hemocytometer. The calculation was performed with the following formula: total microspheres=count (1 corner of 4×4 sections)×(1×104)×(dilution factor)×(resuspension volume in mL). Coupled microspheres were refrigerated at 2-8° C. in the dark.

### Example 2

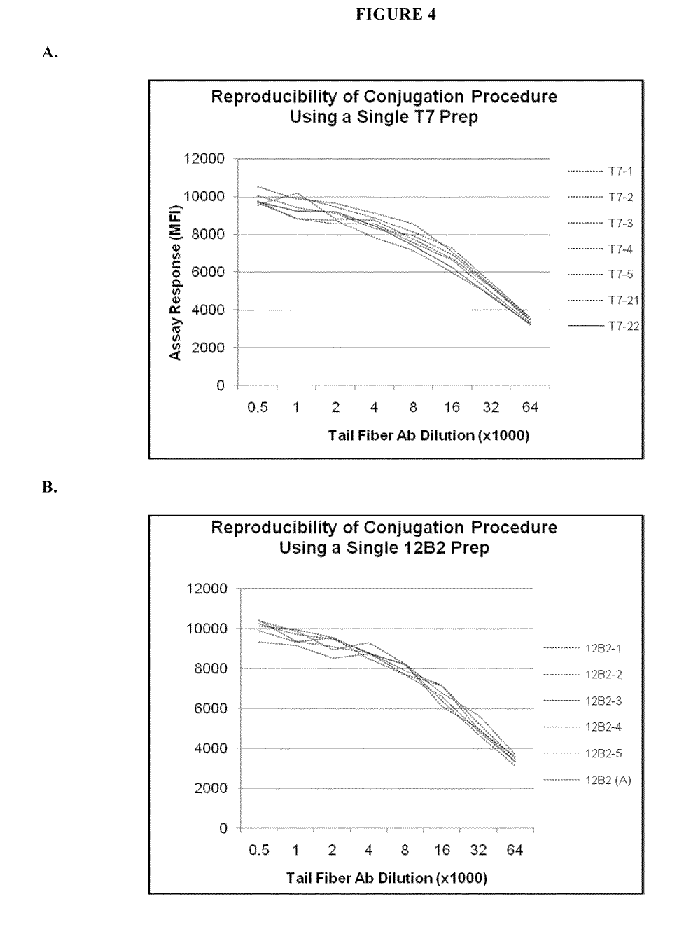

Coupling efficiency was measured with anti T7 tail antibody (1:2000 dilution) and anti-T7 TAG antibody (1:100 dilution). Efficiency of coupling was dependent on pH with a higher pH of 7.2 optimal for yield. The coupling was consistently reproducible as shown in FIG. 4.

### Example 3

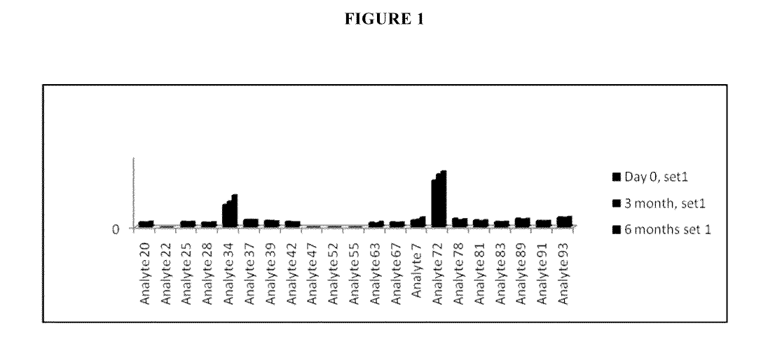

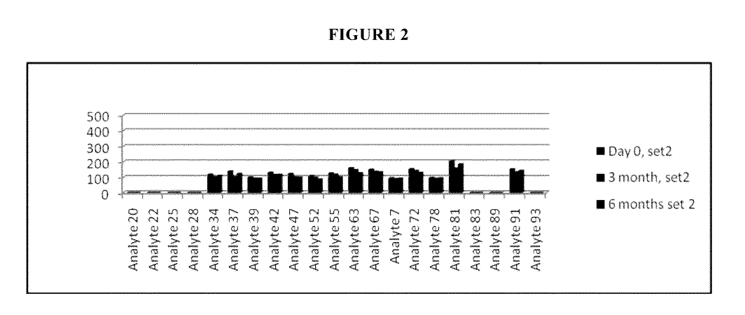

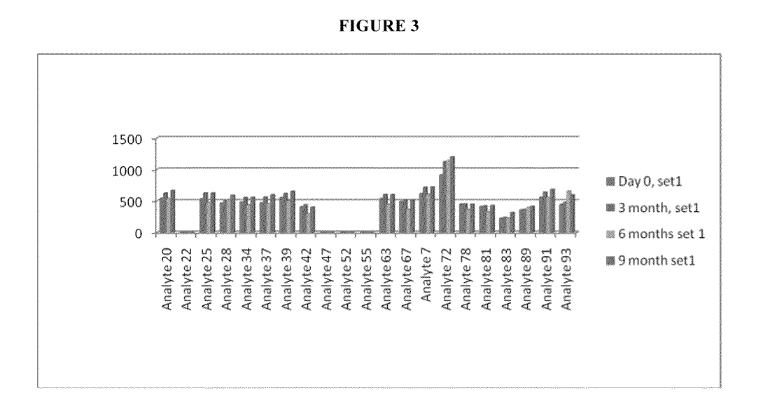

The stability of coupled complex was measured. Stability up to 6 months was confirmed, when tested against various analytes (FIG. 1). Stability up to 6 months was also confirmed when tested against various analytes in a serum-based assay (FIG. 2). Stability up to 9 months was confirmed when tested against various analytes in a serum-based assay (FIG. 3).

### Example 4

Bead-phage conjugates were tested by removing a bead-phage conjugate stock mixture from 4° C. and vortexed to completely resuspend the mixture. An appropriate volume (50 uL per replicate) of diluted beads was prepared in 1× phosphate buffered saline (PBS), 2% bovine serum albumin (BSA), 0.2% Tween-20. An appropriate volume (50 uL per replicate) of each of a 1:50 dilution of control serum (patient sample, pooled serum, stripped serum) was made in 1×PBS, 1.6% polyvinylpyrrolidone (PVP), 1.0% polyvinyl alcohol (PVA), 0.2% Casein. Wells of a filter plate were blocked with 100 uL 1×PBS 1% BSA and aspirated for 10 seconds on a BioTek Elx50 plate washer. To each well was added 50 uL of the diluted beads and diluted antibody and incubated with shaking for 1 hour. The plate wells were washed 5 times in 1×PBS 0.1% Tween-20. Washing was performed with on a BioTek Elx50 plate washer with each wash consisting of the addition of 200 uL of buffer, 5 minutes of shaking, and 30 seconds of aspiration. RM-0126 goat anti-human IgG-PE (Jackson ImmunoResearch Part #109-115-098) was diluted 1:50 in 1×PBS 1% BSA 0.1% Tween-20. To each well, 100 μL of the antibody were added and incubated with shaking for 1 hour. The wells were washed 5 times in 1×PBS 0.1% Tween. Washing was performed with on a BioTek Elx50 plate washer with each wash consisting of the addition of 200 uL of buffer, 5 minutes of shaking, and 30 seconds of aspiration. The Bead-phage conjugates were resuspended with PBS Tween (100 uL/well). The samples were then run in a Luminex analyzer per manufacturer's instruction.

### Example 5

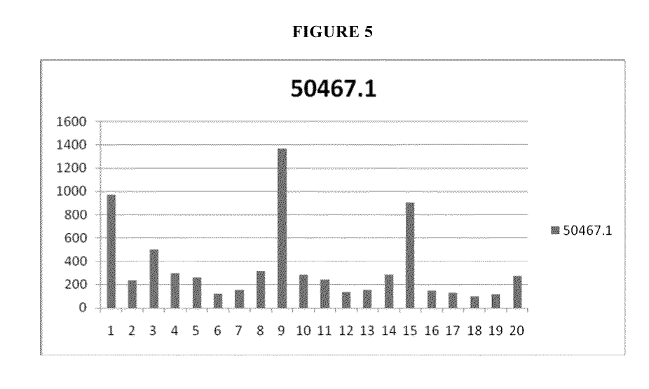

A single human sample (Sample No. 50467.1) was screened against a panel of 20 different biomarkers according to the protocol in Example 4. The Clone ID (Gene Names) of the biomarkers, listed from 1 to 20 along the X-axis in FIG. 5 are: 12B2 (5′-UTR BMI1), 3D10 (RPL34), 1D10 (5′-UTR-BMI1), 4H9 (RPSA), 1B4A (CEP 164), 40A3 (RNA binding protein 6), 21B4 (LAMR1), 3C11 (UTR-Region Chromosome 11), T7, 2E11 (DCHS1), 21H4 (cDNA clone), 4C4 (PSA), 1H5 (RASA1), 15F1 (Nucleolin), 5A1 (H2aa4), 3C4 (5′-UTR BMI1), 2D4 (cDNA clone Chromosome 19), 18D3 (TIMP2), 5F8 (Desmocollin 3), and 2B10 (WDR77). The y-axis represents the signal intensity from the beads linked to the each of the phage-displayed biomarker probes for the sample.

### Example 6

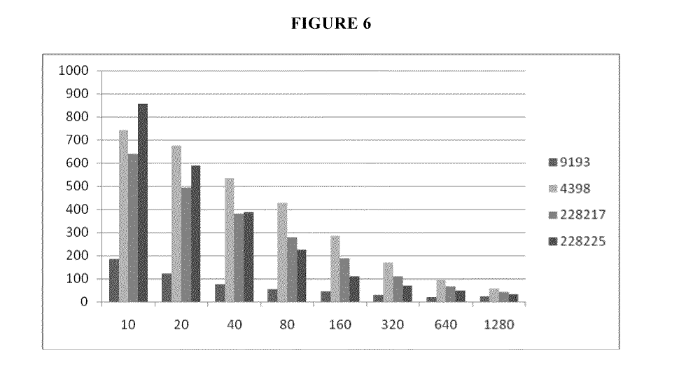

A single biomarker (Clone ID 12B2,5′-UTR BMI1) was used to screen serial dilution series of 4 serum samples (Sample Nos. 9193, 4398, 228217, and 228225) as described in Example 4 to demonstrate the linearity of detection (FIG. 6). The x-axis represents the different dilutions (decreased concentration from left to right), and the y-axis represents the signal intensity from the beads linked to the phage-displayed biomarker probe.

### Example 7

Microsphere Activation:

Stock uncoupled microspheres are resuspended according to the instructions described in the Product Information Sheet provided by Luminex® and 167 μL are transferred to each of 6—1.5 mL microcentrifuge tubes. The tubes are placed in a magnetic rack for about 1 minute, followed by centrifugation at ≧8000×g for 1-2 minutes to pellet. The supernatant is removed and the microspheres are resuspended in 100 μL dH₂O by vortexing and sonication for approximately 20 seconds. The tubes are again placed in a magnetic rack for 1 minute, followed by followed by centrifugation at ≧8000×g for 1-2 minutes to pellet. The supernatant is removed and the microspheres are resuspended and washed in about 1200 μL of 100 mM Monobasic Sodium Phosphate, pH 6.2 by vortexing and sonication for approximately 20 seconds. To the microsphere suspension, 150 μL of 50 mg/mL Sulfo-NHS(N-hydroxysulfosuccinimide; diluted in dH₂O) is added and mixed gently by vortexing. Then, 150 μL of 50 mg/mL EDC (1-Ethyl-3-(3-dimethylaminopropyl)carbodiimide; diluted in dH₂O) is added, followed by gentle mixing by vortex. The microsphere suspension is incubated for about 20 minutes at room temperature; gentle mixing is provided by a rotating mixer. The microsphere suspension is sonicated at 0, 5, 10, and 15 minutes during the incubation step. After the incubation period, the microcentrifuge tubes are placed in a magnetic rack for about 5 minutes followed by centrifugation at ≧8000×g for 1-2 minutes and removal of the supernatant. If the pellet is not compact, about 1 mL of supernatant is removed, the microsphere suspension is placed back in the magnetic rack for 5 minutes and centrifuged as before, and the remainder of the supernatant is removed. The activated microspheres are washed three times according to the following protocol: the microspheres are resuspended in 500 μL of 50 mM MES, pH 5.0 by vortex and sonication for approximately 20 seconds; the microcentrifuge tubes are placed in a magnetic rack for 1 minute followed by centrifugation at ≧8000×g for 1-2 minutes; and the supernatant is removed. The washed and activated microspheres are then resuspended in 150 μL of 50 mM MES, pH 5.0 by vortex and sonication for approximately 20 seconds.

Microsphere-Phage Coupling:

The phage to be coupled to the microsphere is prepared in PBS (Phosphate Buffered Saline) at a concentration of about 0.4 mg/mL. About 100 μL of the phage solution is pre-mixed with about 1250 μL of MES, pH 5.0 and added to 150 μL of the washed activated microspheres. The microsphere-phage coupling reaction is mixed by vortexing and incubated for about 2 hours at room temperature wherein gentle mixing is provided by a rotating mixer. Following this incubation step, the microcentrifuge tubes containing the coupling reaction are placed in a magnetic block for about 5 minutes followed by centrifugation at ≧8000×g for 1-2 minutes and removal of the supernatant. If the pellet is not compact, about 1 mL of supernatant is removed, the microsphere suspension is placed back in the magnetic rack for 5 minutes and centrifuged as before, and the remainder of the supernatant is removed. The phage-coupled microspheres are resuspended in 1.5 mL of Histidine-BSA block (1% bovine serum albumin (BSA), 20 mM histidine, and 0.5 M NaCl) by vortexing. The phage-coupled microspheres are incubated in block for about 30 minutes at room temperature, during which gentle mixing is provided by a rotating mixer. Following blocking, the microcentrifuge tubes containing the phage-coupled microspheres are placed in a magnetic block for about 5 minutes followed by centrifugation at ≧8000×g for 1-2 minutes and removal of the supernatant. If the pellet is not compact, about 1 mL of supernatant is removed, the microsphere suspension is placed back in the magnetic rack for about 5 minutes and centrifuged as before, and the remainder of the supernatant is removed. The phage-coupled microspheres are washed two times according to the following protocol: the phage-coupled microspheres are resuspended in 500 μL of PBS-TBN (Phosphate Buffered Saline-Tween-20, BSA, Sodium Azide) by vortex and sonication for approximately 20 seconds; the microcentrifuge tubes are placed in a magnetic rack for 1 minute followed by centrifugation at ≧8000×g for 1-2 minutes; and the supernatant is removed. The phage-coupled microspheres are then resuspended in 150 μL of Histidine-BSA block and stored at 2-8° C. in the dark.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of producing an antibody detecting complex, the method comprising:
(a) providing a polypeptide probe, wherein the probe is capable of being specifically bound by an antibody, and the probe is present on a phage; and
(b) coupling the phage to a particle, wherein the coupling occurs in a buffer, thereby producing the antibody detecting complex.

2. The method of claim 1, wherein the coupling comprises a linker, wherein the linker covalently couples the particle to the phage.

3. The method of claim 1, wherein the antibody detecting complex is stable for a longer period of time than an antibody complex produced without the buffer.

4. The method of claim 1, wherein the buffer maintains a pH of between about 4 and 6.

5. (canceled)

6. The method of claim 1, wherein the buffer comprises MES (2-(N-morpholino)ethanesulfonic acid).

7. The method of claim 1, wherein the antibody is an autoantibody.

8. The method of claim 7, wherein the autoantibody is cancer autoantibody.

9. The method of claim 8, wherein the cancer autoantibody is prostate cancer autoantibody.

10. (canceled)

11. (canceled)

12. (canceled)

13. (canceled)

14. The method of claim 1, wherein the phage is a t7 phage.

15. The method of claim 1, wherein the particle is a microsphere.

16. (canceled)

17. The method of claim 15, wherein the microsphere comprises identification information.

18. The method of claim 17, wherein the identification information comprises a fluorescent signal.

19. (canceled)

20. (canceled)

21. (canceled)

22. (canceled)

23. (canceled)

24. (canceled)

25. (canceled)

26. A composition for producing an antibody detecting complex comprising:
(a) a polypeptide probe, wherein the probe is capable of being specifically bound by an antibody, and the probe is present on a phage;
(b) a particle; and,
(c) a buffer, wherein the buffer maintains conditions for coupling the phage to the particle to produce the antibody detecting complex.

27. The composition of claim 26, wherein the coupling comprises a linker, wherein the linker covalently couples the particle to the phage.

28. The composition of claim 26, wherein the antibody detecting complex is stable for a longer period of time than an antibody complex produced without the buffer.

29. The composition of claim 26, wherein the buffer maintains a pH of between about 4 and 6.

30. (canceled)

31. The composition of claim 26, wherein the buffer comprises MES (2-(N-morpholino)ethanesulfonic acid).

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. A kit for producing an antibody detecting complex comprising:
(a) a polypeptide probe, wherein the probe is capable of being specifically bound by an antibody, and the probe is present on a phage;
(b) a particle; and,
(c) a buffer, wherein the buffer maintains conditions for coupling the phage to the particle to produce an antibody detecting complex.

52. The kit of claim 51, further comprising:
(d) a linker, wherein the linker covalently couples the particle to the phage.

53. The kit of claim 51, further comprising:
(e) instructions for producing the antibody detecting complex.

54-179. (canceled)
